SEQUENCE LISTING (52 sequences)

SEQ ID NO: 1 - protein, 15 aa, Artificial Sequence
  Description of Artificial Sequence Synthetic peptide
YQGLSTATKDTYDAL

SEQ ID NO: 2 - protein, 23 aa, Artificial Sequence
  Description of Artificial Sequence Synthetic peptide
YQGLSTATKDTYDALHMQALPPR

SEQ ID NO: 3 - protein, 38 aa, Artificial Sequence
  Description of Artificial Sequence Synthetic polypeptide
GMKGERRRGKGHDGLYQGLSTATKDTYDALHMQALPPR

SEQ ID NO: 4 - protein, 28 aa, Artificial Sequence
  Description of Artificial Sequence Synthetic peptide
GHDGLYQGLSTATKDTYDALHMQALPPR

SEQ ID NO: 5 - protein, 54 aa, Artificial Sequence
  Description of Artificial Sequence Synthetic polypeptide
YNELQKDKMAEAYSEIGMKGERRRGKGHDGLYQGLSTATKDTYDALHMQALPPR

SEQ ID NO: 6 - protein, 77 aa, Artificial Sequence
  Description of Artificial Sequence Synthetic polypeptide
DKRRGRDPEMGGKPRRKNPQEGLYNELQKDKMAEAYSEIGMKGERRRGKGHDGLYQGLSTATKDTYDALHMQALPPR

SEQ ID NO: 7 - protein, 66 aa, Artificial Sequence
  Description of Artificial Sequence Synthetic polypeptide
GKPRRKNPQEGLYNELQKDKMAEAYSEIGMKGERRRGKGHDGLYQGLSTATKDTYDALHMQALPPR

SEQ ID NO: 8 - protein, 97 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLHMQALPPR

SEQ ID NO: 9 - protein, 89 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGL

SEQ ID NO: 10 - protein, 74 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEI

SEQ ID NO: 11 - protein, 84 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGK

SEQ ID NO: 12 - protein, 58 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGL

SEQ ID NO: 13 - protein, 35 aa, Artificial Sequence
  Description of Artificial Sequence Synthetic polypeptide
RVKFSRSADAPAYKQGQNQLYNELNLGRREEYDVL

SEQ ID NO: 14 - protein, 46 aa, Artificial Sequence
  Description of Artificial Sequence Synthetic polypeptide
RVKFSRSADAPAYKQGQNQLYNELNLGRREEYDVLDKRRGRDPEMG

SEQ ID NO: 15 - protein, 112 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 240 aa, Artificial Sequence
  Description of Artificial Sequence Synthetic polypeptide
DIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSS

SEQ ID NO: 17 - protein, 55 aa, Artificial Sequence
  Description of Artificial Sequence Synthetic polypeptide
FVPVFLPAKPTTTPAPRPPTPAPTIASQPLSLRPEACRPAAGGAVHTRGLDFACD

SEQ ID NO: 18 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 19 - protein, 28 aa, Artificial Sequence
  Description of Artificial Sequence Synthetic peptide
IYIWAPLAGTCGVLLLSLVITLYCNHRN

SEQ ID NO: 20 - protein, 24 aa, Artificial Sequence
  Description of Artificial Sequence Synthetic peptide
IYIWAPLAGTCGVLLLSLVITLYC

SEQ ID NO: 21 - protein, 22 aa, Artificial Sequence
  Description of Artificial Sequence Synthetic peptide
FWVLVVVGGVLACYSLLVTVAF

SEQ ID NO: 22 - protein, 27 aa, Artificial Sequence
  Description of Artificial Sequence Synthetic peptide
FWVLVVVGGVLACYSLLVTVAFIIFWV

SEQ ID NO: 23 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 24 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 25 - protein, 19 aa, Artificial Sequence
  Description of Artificial Sequence Synthetic peptide
ATNFSLLKQAGDVEENPGP

SEQ ID NO: 26 - protein, 502 aa, Artificial Sequence
  Description of Artificial Sequence Synthetic polypeptide
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEA

SEQ ID NO: 27 - protein, 486 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 490 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 496 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 500 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 488 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 483 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGTTTPAPRPPTPAPTIASQPLSLRPEACRPAAGGAVHTRGLDFACDFWVLVVVGGVLACYSLLVTVAFRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 498 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 493 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDFWVLVVVGGVLACYSLLVTVAFRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 434 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMG

SEQ ID NO: 36 - protein, 432 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMG

SEQ ID NO: 37 - protein, 472 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGK

SEQ ID NO: 38 - protein, 470 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGK

SEQ ID NO: 39 - protein, 520 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGGGGSGGGGSGGGGSGGGGSGHDGLYQGLSTATKDTYDALHMQALPPR

SEQ ID NO: 40 - protein, 510 aa, Artificial Sequence
  Description of Artificial Sequence Synthetic polypeptide
MALPVTALLLPLALLLHAARPDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSGGGGSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSSGFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGGGGSGGGGSGHDGLYQGLSTATKDTYDALHMQALPPR

SEQ ID NO: 41 - DNA, 20 bp, Artificial Sequence
  Description of Artificial Sequence Synthetic oligonucleotide
ttcggaacccaatcactgac

SEQ ID NO: 42 - DNA, 20 bp, Artificial Sequence
  Description of Artificial Sequence Synthetic oligonucleotide
gaggtcaatgtctacggctc

SEQ ID NO: 43 - protein, 20 aa, Artificial Sequence
  Description of Artificial Sequence Synthetic peptide
GGGGSGGGGSGGGGSGGGGS

SEQ ID NO: 44 - protein, 10 aa, Artificial Sequence
  Description of Artificial Sequence Synthetic peptide
GGGGSGGGGS

SEQ ID NO: 45 - protein, 113 aa, Artificial Sequence
  Description of Artificial Sequence Synthetic polypeptide
RVKFSRSADAPAYKQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 724 aa, Artificial Sequence
  Description of Artificial Sequence Synthetic polypeptide
MALPVTALLLPLALLLHAARPDIQMTQSPSSLSASVGDRVTISCRASQDISKYLNWYQQKPGKAPKLLIYHTSRLHSGVPSRFSGSGSGTDYTFTISSLQPEDIATYFCQQGNTLPYTFGGGTKLEIKGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSQVQLQESGPGLVKPSETLSLTCTVSGVSLPDYGVSWIRQPPGKGLEWLGVIWGSETTYYNSALKSRLTISKDTSKSQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 728 aa, Artificial Sequence
  Description of Artificial Sequence Synthetic polypeptide
MALPVTALLLPLALLLHAARPDIQMTQSPSSLSASVGDRVTISCRASQDISKYLNWYQQKPGKAPKLLIYHTSRLHSGVPSRFSGSGSGTDYTFTISSLQPEDIATYFCQQGNTLPYTFGGGTKLEIKGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSQVQLQESGPGLVKPSETLSLTCTVSGVSLPDYGVSWIRQPPGKGLEWLGVIWGSETTYYNSALKSRLTISKDTSKSQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 738 aa, Artificial Sequence
  Description of Artificial Sequence Synthetic polypeptide
MALPVTALLLPLALLLHAARPDIQMTQSPSSLSASVGDRVTISCRASQDISKYLNWYQQKPGKAPKLLIYHTSRLHSGVPSRFSGSGSGTDYTFTISSLQPEDIATYFCQQGNTLPYTFGGGTKLEIKGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSQVQLQESGPGLVKPSETLSLTCTVSGVSLPDYGVSWIRQPPGKGLEWLGVIWGSETTYYNSALKSRLTISKDTSKSQVSLKLSSVTAADTAVYYCAKHYYYGGSYAMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 726 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 730 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 726 aa, Artificial Sequence
  Description of Artificial Sequence Synthetic polypeptide
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 730 aa, Artificial Sequence
  Description of Artificial Sequence Synthetic polypeptide
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSQVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSSGSTSGSGKPGSGEGSTKGDIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKRGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0381233 A1
(43) Pub. Date: Nov. 30, 2023

(54) COMPOSITIONS AND METHODS FOR T CELL ENGINEERING

(71) Applicant: Gracell Biotechnologies (Shanghai) Co., Ltd., Shanghai (CN)

(72) Inventors: JIANNING GE, Shanghai (CN); Xinxin WANG, Shanghai (CN); Chunhui YANG, Shanghai (CN)

(21) Appl. No.: 18/179,792

(22) Filed: Mar. 7, 2023

Related U.S. Application Data
(63) Continuation of application No. PCT/CN2021/116950, filed on Sep. 7, 2021.

(30) Foreign Application Priority Data
Sep. 8, 2020 (WO) PCT/CN2020/114012

(51) Int. Cl. A61K 35/17; C07K 16/28

(52) U.S. Cl. CPC A61K 35/17; C07K 16/2896; C07K 16/2803; C07K 2317/53

(57) ABSTRACT

The present disclosure relates to an engineered immune cell and use thereof. The present disclosure provides an engineered immune cell comprising a CAR or engineered TCR. The engineered immune cells of the present disclosure, when administered into a subject, can inhibit the host immune cells such as T cells and/or NK cells and enhance the survival and persistence of the engineered immune cells in vivo, thereby exhibiting more effective tumor killing activity.

### CROSS-REFERENCE TO RELATED APPLICATIONS

This application is a continuation of International Application No. PCT/CN2021/116950, filed on Sep. 7, 2021, which claims the benefit of International Application No. PCT/CN2020/114012, filed on Sep. 8, 2020, the disclosures of which are hereby incorporated by reference in their entirety.

### SEQUENCE LISTING

The instant application contains a Sequence Listing which has been submitted electronically in TXT file format and is hereby incorporated by reference in its entirety. Said TXT copy, created on May 9, 2023, is named 56758-707_301_SL.txt and is 300,797 bytes in size.

### BACKGROUND

Chimeric antigen receptor T cells (also known as CAR T cells) are T cells that have been genetically engineered to produce an artificial receptor for use in immunotherapy. The first CAR T cells were developed in 1987 by Yoshikazu Kuwana followed in 1989 by Gideon Gross and Zelig Eshhar at Weizmann Institute, Israel.

Chimeric antigen receptors (CARs, also known as chimeric immunoreceptors) are receptor proteins that have been engineered to give T cells the new ability to target a specific protein. The receptors are chimeric because they combine both antigen-binding and T cell activating functions into a single receptor. CARs are composed of four essential regions: an antigen recognition domain, an extracellular hinge region, a transmembrane domain, and an intracellular T cell signaling domain. The sophistication of the engineered CARs has grown over time, and are referred to as first, second, third, and fourth generation CARs depending on their composition.

Generation of tumor-specific CAR T cells is gaining attractions due to its powerful antitumor effects. However, clinical studies have shown that they simultaneously may induce severe side effects such as cytokine release syndrome (CRS).

### SUMMARY

Recognized herein is a need for improved compositions and methods for genetically modifying immune cells for cell therapy. Also recognized herein is a need for modified CARs with improved functions. The present disclosure addresses these needs and provides additional advantages as well.

In one aspect, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 18, and the CD8 transmembrane domain comprises an amino acid sequence of SEQ ID No. 19. In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the CD3ζ intracellular signaling domain is a truncated CD3ζ intracellular signaling domain or a variant of CD3ζ intracellular signaling domain. In some embodiments, the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor α, GPC2, CD70, BAFF-R, TROP-2, and 4-1BB. In some embodiments, the first antigen binding domain binds to CD7. In some embodiments, the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain. In some embodiments, said first antigen binding domain and said second antigen binding domain are arranged, from amino terminus to carboxyl terminus, in one of following patterns (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain. In some embodiments, the CAR further comprises a second hinge domain, a second transmembrane domain, a second costimulatory domain and a second CD3ζ intracellular signaling domain. In some embodiments, the second hinge domain is derived from a polypeptide selected from the group consisting of CD8, CD8α, CD8β, CD27, CD28, CD40, CD45, CD84, CD166, 4-1BB, OX40, ICOS, ICAM-1, CTLA-4, and NKGD2. In some embodiments, the second hinge domain is derived from CD8. In some embodiments, the second hinge domain comprises an amino acid sequence of SEQ ID No. 17 or SEQ ID No. 18. In some embodiments, the second transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2. In some embodiments, the second transmembrane domain is derived from CD8. In some embodiments, the second transmembrane domain comprises an amino acid sequence of SEQ ID No. 19. In some embodiments, the second costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the second CD3ζ intracellular signaling domain is a truncated CD3ζ intracellular signaling domain or a variant of CD3ζ intracellular signaling domain. In some embodiments, the first antigen binding domain and the second antigen binding domain are linked via a linker. In some embodiments, the linker is a cleavable linker. In some embodiments, the linker is a self-cleaving peptide. In some embodiments, the cleavable linker is selected from P2A, T2A, E2A, and F2A. In some embodiments, the engineered immune cell further comprises an enhancer moiety capable of enhancing one or more activities of said engineered immune cell. In some embodiments, the engineered immune cell further comprises further comprises an inducible cell death moiety capable of effecting death of said engineered immune cell upon contacting said chimeric polypeptide with a cell death activator.

In one aspect, provided is an engineered immune cell comprising a chimeric antigen receptor (CAR), wherein the CAR comprises an antigen binding domain that specifically binds to CD7, a CD8 hinge domain, a CD8 transmembrane domain, a costimulatory domain and a CD3ζ intracellular signaling domain, wherein the CD8 hinge domain comprises an amino acid sequence of SEQ ID No. 18, and the CD8 transmembrane domain comprises an amino acid sequence of SEQ ID No. 19. In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the costimulatory domain is derived from 4-1BB. In some embodiments, the CD3ζ intracellular signaling domain is a truncated CD3ζ intracellular signaling domain or a variant of CD3ζ intracellular signaling domain.

In one aspect, provided is an engineered immune cell comprising a chimeric antigen receptor (CAR), wherein the CAR comprises an antigen binding domain, a CD8 hinge domain, a CD8 transmembrane domain, a 4-1BB costimulatory domain and a CD3ζ intracellular signaling domain, wherein the CD8 hinge domain comprises an amino acid sequence of SEQ ID No. 18, and the CD8 transmembrane domain comprises an amino acid sequence of SEQ ID No. 19. In some embodiments, the CD3ζ intracellular signaling domain is a truncated CD3ζ intracellular signaling domain or a variant of CD3ζ intracellular signaling domain. In some embodiments, the CAR comprises a first antigen binding domain and a second antigen binding domain. In some embodiments, the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain. In some embodiments, said first antigen binding domain and said second antigen binding domain are arranged, from amino terminus to carboxyl terminus, in one of following patterns (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain. In some embodiments, the first antigen binding domain and the second antigen binding domain are linked via a linker. In some embodiments, the CAR further comprises a second hinge domain, a second transmembrane domain, a second costimulatory domain and a second CD3ζ intracellular signaling domain.

In one aspect, provided is an engineered immune cell comprising a chimeric antigen receptor (CAR), wherein the CAR comprises an antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 17, the CD8 transmembrane domain comprises an amino acid sequence of SEQ ID No. 19. In some embodiments, the CD3ζ intracellular signaling domain is a truncated CD3ζ intracellular signaling domain or a variant of CD3ζ intracellular signaling domain. In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7. In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the costimulatory domain is derived from 4-1BB.

In one aspect, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In one aspect, provided is an engineered immune cell, comprising: (i) an enhancer moiety capable of enhancing one or more activities of said engineered immune cell; (ii) a chimeric antigen receptor (CAR) comprising an antigen binding domain, a hinge domain, a transmembrane domain, a costimulatory domain and a truncated CD3ζ intracellular signaling domain, wherein the truncated CD3ζ intracellular signaling domain is devoid of ITAM3 or a fragment thereof.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

In some embodiments, the hinge domain is derived from a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

In some embodiments, the transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the ITAM3 comprises an amino acid sequence of YXXL/I-X₇-YXXL/I, and each X is independently any amino acid.

In some embodiments, the truncated CD3ζ intracellular signaling domain is devoid of an amino acid sequence of SEQ ID NO.1 or a fragment thereof. In some embodiments, the truncated CD3ζ intracellular signaling domain is devoid of an amino acid sequence of SEQ ID NO.2 or a fragment thereof. In some embodiments, the truncated CD3ζ intracellular signaling domain is devoid of an amino acid sequence of SEQ ID NO.3 or a fragment thereof. In some embodiments, the truncated CD3ζ intracellular signaling domain is devoid of an amino acid sequence of SEQ ID NO.4 or a fragment thereof. In some embodiments, the truncated CD3ζ intracellular signaling domain comprises at least ITAM1 and ITAM2. In some embodiments, the ITAM1 comprises an amino acid sequence of YXXL/I-X₇-YXXL/I, and each X is independently any amino acid. In some embodiments, the ITAM2 comprises an amino acid sequence of YXXL/I-X₈-YXXL/I, and each X is independently any amino acid. In some embodiments, the truncated CD3ζ intracellular signaling domain comprises an amino acid sequence of SEQ ID NO. 8. In some embodiments, the truncated CD3ζ intracellular signaling domain comprises an amino acid sequence of SEQ ID NO. 9. In some embodiments, the truncated CD3ζ intracellular signaling domain comprises an amino acid sequence of SEQ ID NO. 10. In some embodiments, the truncated CD3ζ intracellular signaling domain comprises an amino acid sequence of SEQ ID NO. 11.

In some embodiments, said enhancer moiety is configured to constitutively upregulate one or more intracellular signaling pathways of said engineered immune cell. In some embodiments, said one or more intracellular signaling pathways are one or more cytokine signaling pathways. In some embodiments, said enhancer moiety is self-activating through self-oligomerizing. In some embodiments, said enhancer moiety is self-activating through self-dimerizing. In some embodiments, said enhancer moiety is a cytokine or a cytokine receptor. In some embodiments, said enhancer moiety is selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof. In some embodiments, said enhancer moiety functions as a trans-activating factor or a cis-activating factor.

In some embodiments, the enhancer is linked to the CAR via a linker. In some embodiments, the linker is a cleavable linker. In some embodiments, the linker is a self-cleaving peptide. In some embodiments, the cleavable linker is selected from P2A, T2A, E2A, and F2A.

In some embodiments, the engineered immune cell further comprises an inducible cell death moiety capable of effecting death of said engineered immune cell upon contacting said chimeric polypeptide with a cell death activator. In some embodiments, said enhancer moiety is linked to said inducible cell death moiety. In some embodiments, said inducible cell death moiety is selected from the group consisting of rapaCasp9, iCasp9, HSV-TK, ACD20, mTMPK, ACD19, RQR8, Her2t, CD30, BCMA and EGFRt. In some embodiments, said inducible cell death moiety is EGFRt, and said cell death activator is an antibody or an antigen binding fragment thereof that binds EGFRt. In some embodiments, said inducible cell death moiety is HSV-TK, and said cell death activator is GCV. In some embodiments, said inducible cell death moiety is iCasp9, and said cell death activator is AP1903. In some embodiments, said cell death activator comprises a nucleic acid, a polynucleotide, an amino acid, a polypeptide, lipid, a carbohydrate, a small molecule, an enzyme, a ribosome, a proteasome, a variant thereof, or any combination thereof.

In some embodiments, the engineered immune cell is a T cell, an NKT cell or an NK cell. In some embodiments, the T cell is an alpha beta T cell or a gamma delta T cell. In some embodiments, the engineered immune cell is derived from a stem cell. In some embodiments, the stem cell is a hematopoietic stem cell (HSC) or an induced pluripotent stem cell (iPSC). In some embodiments, the engineered immune cell is an autologous cell or an allogeneic cell. In some embodiments, the engineered immune cell is obtained from a subject having a condition. In some embodiments, the engineered immune cell is obtained from a healthy donor.

In another aspect, provided is an engineered immune cell, comprising: (i) an enhancer moiety capable of enhancing one or more activities of said engineered immune cell; (ii) a chimeric antigen receptor (CAR) comprising an antigen binding domain, a hinge domain, a transmembrane domain, a costimulatory domain and a variant of CD3ζ intracellular signaling domain, wherein the variant of CD3ζ intracellular signaling domain comprises a loss of function mutation.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

In some embodiments, the hinge domain is derived from a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

In some embodiments, the transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the variant of CD3ζ intracellular signaling domain comprises two loss of function mutations. In some embodiments, the loss of function mutation is present in an ITAM domain of CD3ζ intracellular signaling domain. In some embodiments, the loss of function mutation is present in ITAM1, ITAM2, or ITAM3 of CD3ζ intracellular signaling domain. In some embodiments, the loss of function mutation is present at Tyrosine residue of ITAM domain of CD3ζ intracellular signaling domain. In some embodiments, the Tyrosine residue is substituted with phenylalanine. In some embodiments, the loss of function mutation is present at Leucine residue of ITAM domain of CD3ζ intracellular signaling domain. In some embodiments, the loss of function mutation is present in a non-ITAM region of CD3ζ intracellular signaling domain.

In another aspect, provided is an engineered immune cell, comprising: (i) a functionally inactive T cell receptor (TCR), and (ii) a chimeric antigen receptor (CAR) comprising an antigen binding domain, a hinge domain, a transmembrane domain, a costimulatory domain and a truncated CD3ζ intracellular signaling domain, wherein the truncated CD3ζ intracellular signaling domain is devoid of ITAM3.

In another aspect, provided is an engineered immune cell, comprising: (i) a functionally inactive T cell receptor (TCR), and (ii) a chimeric antigen receptor (CAR) comprising an antigen binding domain, a hinge domain, a transmembrane domain, a costimulatory domain and a variant of CD3ζ intracellular signaling domain, wherein the variant of CD3ζ intracellular signaling domain comprises a loss of function mutation.

In another aspect, provided is an engineered immune cell, comprising: (i) an enhancer moiety capable of enhancing one or more activities of said engineered immune cell; (ii) a chimeric antigen receptor (CAR) comprising an antigen binding domain that specifically binds to CD7, a hinge domain, a transmembrane domain, a costimulatory domain and a truncated CD3ζ intracellular signaling domain, wherein the truncated CD3ζ intracellular signaling domain is devoid of ITAM3.

In another aspect, provided is an engineered immune cell, comprising: (i) an enhancer moiety capable of enhancing one or more activities of said engineered immune cell; (ii) a chimeric antigen receptor (CAR) comprising an antigen binding domain that specifically binds to CD7, a hinge domain, a transmembrane domain, a costimulatory domain and a variant of CD3ζ intracellular signaling domain, wherein the variant of CD3ζ intracellular signaling domain comprises a loss of function mutation.

In another aspect, provided is a chimeric antigen receptor (CAR) comprising an antigen binding domain, a CD8 hinge domain, a CD8 transmembrane domain, a costimulatory domain and a truncated CD3ζ intracellular signaling domain, wherein the CD8 hinge domain comprises an amino acid sequence of SEQ ID No. 17, the CD8 transmembrane domain comprises an amino acid sequence of SEQ ID No. 19, and the truncated CD3ζ intracellular signaling domain is devoid of ITAM3 or a fragment thereof.

In some embodiments, the truncated CD3ζ intracellular signaling domain comprises an amino acid sequence of SEQ ID NO. 11.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7.

In another aspect, provided is a chimeric antigen receptor (CAR) comprising an antigen binding domain, a CD8 hinge domain, a CD8 transmembrane domain, a costimulatory domain and a variant of CD3ζ intracellular signaling domain, wherein the CD8 hinge domain comprises an amino acid sequence of SEQ ID No. 17, the CD8 transmembrane domain comprises an amino acid sequence of SEQ ID No. 19, and the variant of CD3ζ intracellular signaling domain comprises a loss of function mutation.

In some embodiments, the variant of CD3ζ intracellular signaling domain comprises two loss of function mutations. In some embodiments, the loss of function mutation is present in an ITAM domain of CD3ζ intracellular signaling domain. In some embodiments, the loss of function mutation is present in ITAM1, ITAM2, or ITAM3 of CD3ζ intracellular signaling domain. In some embodiments, the loss of function mutation is present at Tyrosine residue of ITAM domain of CD3ζ intracellular signaling domain. In some embodiments, the Tyrosine residue is substituted with phenylalanine. In some embodiments, the loss of function mutation is present at Leucine residue of ITAM domain of CD3ζ intracellular signaling domain. In some embodiments, the loss of function mutation is present in a non-ITAM region of CD3ζ intracellular signaling domain.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7.

In another aspect, provided is a chimeric antigen receptor (CAR) comprising an antigen binding domain, a CD8 hinge domain, a CD8 transmembrane domain, a 4-1BB costimulatory domain and a truncated CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the truncated CD3ζ intracellular signaling domain is devoid of ITAM3 or a fragment thereof.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7.

In another aspect, provided is a chimeric antigen receptor (CAR) comprising an antigen binding domain, a CD8 hinge domain, a CD8 transmembrane domain, a 4-1BB costimulatory domain and a variant of CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the variant of CD3ζ intracellular signaling domain comprises a loss of function mutation. In some embodiments, the variant of CD3ζ intracellular signaling domain comprises two loss of function mutations. In some embodiments, the loss of function mutation is present in an ITAM domain of CD3ζ intracellular signaling domain. In some embodiments, the loss of function mutation is present in ITAM1, ITAM2, or ITAM3 of CD3ζ intracellular signaling domain. In some embodiments, the loss of function mutation is present at Tyrosine residue of ITAM domain of CD3ζ intracellular signaling domain. In some embodiments, the Tyrosine residue is substituted with phenylalanine. In some embodiments, the loss of function mutation is present at Leucine residue of ITAM domain of CD3ζ intracellular signaling domain. In some embodiments, the loss of function mutation is present in a non-ITAM region of CD3ζ intracellular signaling domain.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

In some embodiments, the antigen binding domain binds to CD7.

In another aspect, provided, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the CD3ζ intracellular signaling domain is a truncated CD3ζ intracellular signaling domain or a variant of CD3ζ intracellular signaling domain.

In some embodiments, the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the first antigen binding domain binds to CD7.

In some embodiments, the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

In some embodiments, said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

In some embodiments, said first antigen binding domain and said second antigen binding domain are arranged, from amino terminus to carboxyl terminus, in one of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

In some embodiments, the CAR further comprises a second hinge domain, a second transmembrane domain, a second costimulatory domain and a second CD3ζ intracellular signaling domain.

In some embodiments, the second hinge domain is derived from a polypeptide selected from the group consisting of CD8, CD8α, CD8β, CD27, CD28, CD40, CD45, CD84, CD166, 4-1BB, OX40, ICOS, ICAM-1, CTLA-4, and NKGD2. In some embodiments, the second hinge domain is derived from CD8. In some embodiments, the second hinge domain comprises an amino acid sequence of SEQ ID No. 17.

In some embodiments, the second transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2. In some embodiments, the second transmembrane domain is derived from CD8. In some embodiments, the second transmembrane domain comprises an amino acid sequence of SEQ ID No. 19.

In some embodiments, the second costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the second CD3ζ intracellular signaling domain is a truncated CD3ζ intracellular signaling domain or a variant of CD3ζ intracellular signaling domain.

In some embodiments, the first antigen binding domain and the second antigen binding domain are linked via a linker. In some embodiments, the linker is a cleavable linker. In some embodiments, the linker is a self-cleaving peptide. In some embodiments, the cleavable linker is selected from P2A, T2A, E2A, and F2A.

In some embodiments, the engineered immune cell further comprises an enhancer moiety capable of enhancing one or more activities of said engineered immune cell.

In some embodiments, the engineered immune cell further comprises further comprises an inducible cell death moiety capable of effecting death of said engineered immune cell upon contacting said chimeric polypeptide with a cell death activator.

In another aspect, provided is an engineered immune cell comprising a chimeric antigen receptor (CAR), wherein the CAR comprises an antigen binding domain that specifically binds to CD7, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the costimulatory domain is derived from 4-1BB.

In some embodiments, the CD3ζ intracellular signaling domain is a truncated CD3ζ intracellular signaling domain or a variant of CD3ζ intracellular signaling domain.

In another aspect, provided is an engineered immune cell comprising a chimeric antigen receptor (CAR), wherein the CAR comprises an antigen binding domain, a CD8 hinge domain, a CD8 transmembrane domain, a 4-1BB costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the CD3ζ intracellular signaling domain is a truncated CD3ζ intracellular signaling domain or a variant of CD3ζ intracellular signaling domain.

In some embodiments, the CAR comprises a first antigen binding domain and a second antigen binding domain. In some embodiments, the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

In some embodiments, said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

In some embodiments, said first antigen binding domain and said second antigen binding domain are arranged, from amino terminus to carboxyl terminus, in one of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

In some embodiments, the first antigen binding domain and the second antigen binding domain are linked via a linker.

In some embodiments, the CAR further comprises a second hinge domain, a second transmembrane domain, a second costimulatory domain and a second CD3ζ intracellular signaling domain.

In another aspect, provided is an engineered immune cell comprising a chimeric antigen receptor (CAR), wherein the CAR comprises an antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 18, the CD8 transmembrane domain comprises an amino acid sequence of SEQ ID No. 19.

In some embodiments, the CD3ζ intracellular signaling domain is a truncated CD3ζ intracellular signaling domain or a variant of CD3ζ intracellular signaling domain.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7.

In some embodiments, the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83. In some embodiments, the costimulatory domain is derived from 4-1BB.

In another aspect, provided is an engineered immune cell comprising a chimeric antigen receptor (CAR), wherein the CAR comprises an antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

In some embodiments, the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB. In some embodiments, the antigen binding domain binds to CD7.

In another aspect, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 18, and the CD8 transmembrane domain comprises an amino acid sequence of SEQ ID No. 19.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A, FIG. 1B, FIG. 1C and FIG. 1D illustrate exemplary designs of CARs with modified structures of the present application.

FIG. 2 illustrates in vitro killing activity of CAR T cells with modified structures of the present application.

FIG. 3 illustrates releases of cytokines by CAR T cells with modified structures of the present application.

FIG. 4 illustrates in vivo anti-tumor activities of the CAR T cells in mice.

FIG. 5 illustrates expression of dual CAR with modified structures of the present application in T cells.

FIG. 6 illustrates killing efficacy of dual CAR T cells with modified structures of the present application against Hela cells expressing CD7 or CD19.

FIG. 7 illustrates killing efficacy of dual CAR T cells with modified structures of the present application against CCRF and Nalm6 cells.

FIG. 8 illustrates releases of cytokines by dual CAR T cells with modified structures of the present application.

FIG. 9 illustrates flow cytometry graphs showing CAR expression in CAR T cells with modified structures of 18-21. Eight days after transduction, all CAR T cells exhibited good CD19 CAR and CD7 CAR expressions as measured by CD19 or CD7 antigens.

FIG. 10 illustrates cell killing efficacy of dual CAR T with modified structures 18-21 as measured from luciferase assay. CCRF-CEM cell (acute lymphoblastic leukemia cell line), Nalm6 cell (B cell precursor leukemia cell line), or primary T cell was mixed with dual CAR T cell comprising the modified structures 18-21 at a ratio that is 5:1, 1:1, or 1:5. Cell killing efficacy was measured at multiple time points.

FIG. 11a and FIG. 11b illustrate releases of cytokines by dual CAR T cells with modified structures 18-21 upon killing of the cancer cells (FIG. 11a, CCRF cells; and FIG. 11b, Nalm6 cells). The cytokine measurements were conducted with the CCRF-CEM cell or Nalm6 cell mixed with the dual CAR T cell at a 1:1 ratio after 24 hours. The cytokines were measured with cytometric bead array (CBA).

FIG. 12 illustrates proliferation of dual CAR T cell with modified structures 18-21 stimulated by mixing the dual CAR T cell with either CCRF-CEM cell or Nalm6 cell at a 1:3 ratio. The proliferation of dual CAR T cell was measured after each round of killing of the CCRF-CEM cell or Nalm6 cell.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definition

The term “about,” as used herein, refers to a value or composition within a range of acceptable tolerances for a particular value or composition as determined, which will depend in part on how the value or composition is measured or measured, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “administering,” as used herein, refers to physically introducing a product of the present disclosure into a subject using any of a variety of methods and delivery systems, including intravenous, intramuscular, subcutaneous, intraperitoneal, spinal or other routes of parenteral administration, for example by injection or infusion.

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen. In some cases, an antigen may be bound to a substrate (e.g., a cell membrane). Alternatively, an antigen may not be bound to a substrate (e.g., a secreted molecule, such as a secreted polypeptide).

The term “antibody (Ab),” as used herein, include, but is not limited to, an immunoglobulin that specifically binds to an antigen and comprises at least two heavy (H) chains and two light (L) chains interconnected by disulfide bonds, or a fragment thereof. Each H chain comprises a heavy chain variable region (abbreviated herein as VH) and a heavy chain constant region. The heavy chain constant region comprises three constant domains CH1, CH2 and CH3. Each light chain comprises a light chain variable region (abbreviated herein as VL) and a light chain constant region. The light chain constant region comprises a constant domain CL. The VH and VL regions can be further subdivided into hypervariable regions called complementarity determining regions (CDRs) interspersed with more conserved regions called framework regions (FR). Each VH and VL contains three CDRs and four FRs, arranged from the amino terminus to the carboxy terminus in the following order: FR1, CDR1, FR2, CDR2, FR3, CDR3, FR4. The variable regions of the heavy and light chains contain a binding domain that interacts with the antigen.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-S-UTP, Rhodamine Green-S-dUTP, tetramethylrhodamine-6-UTP, tetramethylrhodamine-6-dUTP, Texas Red-S-UTP, Texas Red-S-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “endogenous,” as used herein, refers to a nucleic acid molecule or polypeptide normally expressed in a cell or tissue.

The term “exogenous,” as used herein, refers to the nucleic acid molecule or polypeptide is not endogenously present in the cell or is present at a level sufficient to achieve the functional effects obtained upon overexpression. Thus, the term “exogenous” includes any recombinant nucleic acid molecule or polypeptide expressed in a cell, e.g., a foreign, heterologous, and overexpressed nucleic acid molecule and polypeptide.

The term “autologous” as used herein, refers to origination from the same being. For example, an autologous sample (e.g., cells) can refer to a sample which is removed, processed, and then given back to the same subject (e.g., patient) at a later time. Autologous, with respect to a process, can be distinguished from an allogenic process in which the donor of a sample (e.g., cells) and the recipient of the sample are not the same subject.

The term “allogeneic” as used herein, refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” as used herein, refers to a graft derived from an animal of a different species.

The term “T cell and NK cell consensus marker,” as used herein, refers to a marker co-existing on T cells and NK cells, including but not limited to: CD2, CD7, CD38, CD45, CD48, CD50, CD52, CD56, CD69, CD100, CD122, CD132, CD161, CD159a, CD159c, CD314.

The term “marker of T cells and/or NK cells,” as used herein, refers to markers present in T cells or NK cells, respectively, or both T cells and NK cells, including but not limited to: CD2, CD3, CD4, CD5, CD7, CD8, CD16a, CD16b, CD25, CD27, CD28, CD38, CD45, CD48, CD50, CD52, CD56, CD57, CD62L, CD69, CD94, CD100, CD102, CD122, CD127, CD132, CD160, CD161 CD178, CD218, CD226, CD244, CD159a (NKG2A), CD159c (NKG2C), NKG2E, CD314 (NKG2D), CD305, CD335 (NKP46), CD337, SLAMF7.

As used herein, the term “native” in the context of a protein or polypeptide refers to a naturally occurring amino acid sequence, including immature or precursor and mature forms of the protein or polypeptide.

As used herein, the term “modified” or “modification” in the context of a protein or polypeptide, refers to a protein or polypeptide having an addition, deletion, substitution (or mutation) of one or more amino acids as compared to the sequence of the native polypeptide.

In some embodiments, the modified polypeptide comprises a polypeptide having an addition of one or more amino acids as compared to the sequence of the native polypeptide. Unless otherwise specified, the modified polypeptide may comprise a polypeptide having an addition of at least 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 amino acids as compared to the sequence of the native polypeptide. Unless otherwise specified, the modified polypeptide may comprise a polypeptide having an addition of at least 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 continuous amino acids from the N terminus or C terminus as compared to the sequence of the native polypeptide, and in this case, the modified polypeptide may also be referred as an extended polypeptide (or “having an extension”) herein.

In some embodiments, the modified polypeptide comprises a polypeptide having a deletion of one or more amino acids as compared to the sequence of the native polypeptide. Unless otherwise specified, the modified polypeptide may comprise a polypeptide having a deletion of at least 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 amino acids as compared to the sequence of the native polypeptide. Unless otherwise specified, the modified polypeptide may comprise a polypeptide having a deletion of at least 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 continuous amino acids from the N terminus or C terminus as compared to the sequence of the native polypeptide, and in this case, the modified polypeptide may also be referred as a “truncated polypeptide” (or “having a truncation”) herein.

In some embodiments, the modified polypeptide comprises a polypeptide having a substitution (or mutation) of one or more amino acids as compared to the sequence of the native polypeptide. The polypeptide having a substitution (or mutation) of one or more amino acids may also be referred as “a variant of the polypeptide” herein. Unless otherwise specified, the modified polypeptide may comprise a polypeptide having a substitution (or mutation) of at least 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 amino acids as compared to the sequence of the native polypeptide. In some embodiments, the substitution (or mutation) may result in functional inactivation or loss of function of the polypeptide.

The term “functionally inactivate,” “functional inactivation” or “loss of function” as used herein refers to that a functional gene or the product of the gene such as mRNA or protein is prevented or inhibited. The inactivation may be achieved by deletion, addition or substitution of the gene or the promoter thereof, so that expression does not occur, or mutation of the coding sequence of the gene so that the gene product such as mRNA or protein is inactive. The functional inactivation may be complete or partial. Inactivation of a gene can encompass all degrees of inactivation, including gene silencing, knockout, inhibition and disruption. In some embodiments, the functional inactivation is introduced by CRISPR-Cas9 system.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more conditions (e.g., diseases or symptoms) under treatment. For prophylactic benefit, a composition can be administered to a subject at risk of developing a particular condition, or to a subject reporting one or more of the physiological symptoms of a disease, even though the condition may not have yet been manifested.

### Overview

CARs can comprise an extracellular antigen recognition region, for example, a scFv (single-chain variable fragment) or a single domain antibody (sdAb, such as a VHH antibody), a hinge region (such as CD8 hinge region), a transmembrane domain (such as CD8 or CD28 transmembrane domain), an costimulatory domain (such as 4-1BB or CD28 costimulatory domain) and an intracellular signaling region (such as CD3ζ). The extracellular domain of CARs can recognize a specific antigen and then transduce the signal through the intracellular domain, causing T cell activation and proliferation, cytolysis toxicity, and secretion of cytokines, thereby eliminating target cells. The patient's autologous T cells (or heterologous donors) can be first isolated, activated and genetically engineered to produce CAR T cells, which can be then injected into the same patient. In this way, the probability of graft-versus-host disease may be reduced, and the antigen can be recognized by T cells in a non-MHC-restricted manner.

The present disclosure provides compositions and methods to engineer a cell, e.g., an immune cell, such that it can comprise a modified CAR structure with desired activities as well as capabilities of targeting disease-associated antigen (e.g., tumor-associated antigen or tumor cell marker such as BCMA, CD19) and, optionally, an immune cell antigen (e.g., CD7, CD137) through single, bispecific or multivalent CAR(s).

For example, the present disclosure provides an engineered immune cell comprising a CAR with modified CD3ζ and capable of targeting a tumor cell marker and an immune cell antigen such as CD7. The present disclosure also provides an engineered immune cell comprising a CAR with modified hinge region and/or modified transmembrane domain and capable of targeting a tumor cell marker and an immune cell antigen such as CD7. The present disclosure also provides an engineered immune cell comprising a CAR with regular hinge region and/or modified transmembrane domain and capable of targeting a tumor cell marker and an immune cell antigen such as CD7. Also provided is an engineered immune cell comprising a CAR with modified hinge region and/or modified transmembrane domain, modified CD3ζ and capable of targeting a tumor cell marker and an immune cell antigen such as CD7.

The present disclosure provides an engineered immune cell comprising a bispecific CAR with modified CD3ζ. The present disclosure also provides an engineered immune cell comprising a bispecific CAR with modified hinge region and/or modified transmembrane domain. The present disclosure also provides an engineered immune cell comprising a bispecific CAR with regular hinge region and/or modified transmembrane domain. Also provided is an engineered immune cell comprising a bispecific CAR with modified hinge region and/or modified transmembrane domain, modified CD3ζ.

The endogenous TCR of the immune cell can be inactivated (e.g., disrupted, inhibited, knocked out or silenced). The CAR T of the present disclosure which targets the tumor cell marker and the immune cell antigen can eliminate positive tumor cells and clear host immune cell antigen positive T and NK cells, thereby avoiding host rejection (HVG).

In the present disclosure, optionally, the endogenous MHC molecule can be inactivated (e.g., disrupted, inhibited, knocked out or silenced) to avoid the host rejection. The endogenous expression of the immune cell antigen in the engineered immune cell can be inactivated (e.g., disrupted, inhibited, knocked out or silenced) to avoid fratricide. The endogenous TCR of the engineered immune cell can be knocked out, and graft-versus-host disease (GVHD) can be prevented, thereby preparing a general-purpose or universal CAR T (UCAR-T) cell. The engineered immune cell can be derived from an autologous T cell or an allogeneic T cell.

In some cases, the engineered immune cell can further comprise an enhancer moiety. The enhancer moiety can regulate one or more activities of the engineered immune cell when the engineered immune cell is administered to a subject. For example, the enhancer moiety can be a cytokine (e.g., IL-5 or IL-7) or a cytokine receptor (e.g., IL-5R or IL-7R). The enhancer moiety can enhance a signaling pathway within the engineered immune cell, for example, STAT5 signaling pathway.

The engineered immune cell can further comprise an inducible cell death moiety such as a truncated epidermal growth factor receptor (EGFRt or tEGFR, which can be used interchangeably herein; see U.S. Pat. No. 9,447,194B2 and PCT Publication No. WO2018038945).

The inducible cell death moiety or the enhancer moiety can be introduced in the immune cell via a separate expression vector. In some cases, the inducible cell death moiety and the enhancer moiety may be introduced into the immune cell via an expression vector comprising sequences encoding both moieties. In some cases, the inducible cell death moiety and the enhancer moiety are linked and are expressed as a chimeric polypeptide.

The application of the engineered immune cells provided herein in cell therapy can treat the disease (e.g., cancer) of a patient with improved functions, be prepared in large-scale in advance to avoid GVHD and HvG, reduce treatment costs, inactivate CAR T at any time if necessary, reduce side effects of immunotherapy such as cytokine release syndrome, and ensure product safety. The engineered cells provided herein can be referred to as universal CAR T cells (UCAR T cells).

### Chimeric Antigen Receptor (CAR)

The cell (e.g., immune cell or engineered immune cell) provided herein can comprise one or more CARs. The CAR can include an antigen binding domain, a hinge region, a transmembrane domain, a costimulatory domain and an intracellular signaling region. The extracellular domain of CAR can include an antigen binding domain, and a hinge region. The intracellular domain can include a costimulatory domain and a signaling region.

### Hinge Region

A hinge region, also referred to as a spacer, is a portion of the extracellular region of CAR which links the extracellular antigen-binding domain to the transmembrane domain. Hinges can affect the overall performance of CAR T cells. The hinge region can supply stability for efficient CAR expression and activity and provide flexibility to access the targeted antigen. The length of the spacer is crucial to provide adequate intercellular distance for immunological synapse formation, highlighting the need to optimize hinge accordingly.

Any suitable hinge region known in the art can be used in the CAR of the present application. In some embodiments, the hinge region of the CAR of present application can be derived from a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

In some embodiments, the hinge region of the CAR of the present application can be a native hinge region of a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

In some embodiments, the hinge region of the present application can be a modified hinge region of a polypeptide selected from the group consisting of CD8, CD8α, CD8β, CD27, CD28, CD40, CD45, CD84, CD166, 4-1BB, OX40, ICOS, ICAM-1, CTLA-4, and NKGD2.

In some embodiments, the hinge region of the CAR of the present application can be derived from CD8. In some embodiments, the hinge region of the CAR of the present application can be the hinge region of native CD8. In some embodiments, the hinge region of the CAR of the present application can be a modified hinge region of CD8. In some embodiments, the hinge region of the CAR of the present application can comprise an extended region as compared to the native hinge region of CD8. In some embodiments, the hinge region of the CAR of the present application can comprise a truncated hinge region as compared to the native hinge region of CD8.

In some embodiments, the hinge region of the CAR of the present application comprises an amino acid sequence having at least 75%, at least 80%, at least 85%, at least 90%, at least 95%, at least 96%, at least 97%, at least 98%, or at least 99% identity with SEQ ID No. 17. In some embodiments, the hinge region of the CAR of the present application comprises an amino acid sequence of SEQ ID No. 17. In some embodiments, the hinge region of the CAR of the present application comprises an amino acid sequence having one or more amino acid modifications as compared to SEQ ID No. 17. In some embodiments, the hinge region of the CAR of the present application comprises an amino acid sequence having 1, 2, 3, 4, or 5 amino acid modifications as compared to SEQ ID No. 17. In some embodiments, the hinge region of the CAR of the present application comprises an amino acid sequence having one or more amino acid additions, deletions, mutations as compared to SEQ ID No. 17. In some embodiments, the hinge region of the CAR of the present application comprises an amino acid sequence having 1, 2, 3, 4 or 5 amino acid additions, deletions, mutations as compared to SEQ ID No. 17.

In some embodiments, the hinge region of the CAR of the present application comprises an amino acid sequence having at least 75%, at least 80%, at least 85%, at least 90%, at least 95%, at least 96%, at least 97%, at least 98%, or at least 99% identity with SEQ ID No. 18. In some embodiments, the hinge region of the CAR of the present application comprises an amino acid sequence of SEQ ID No. 18. In some embodiments, the hinge region of the CAR of the present application comprises an amino acid sequence having one or more amino acid modifications as compared to SEQ ID No. 18. In some embodiments, the hinge region of the CAR of the present application comprises an amino acid sequence having 1, 2, 3, 4 or 5 amino acid modifications as compared to SEQ ID No. 18. In some embodiments, the hinge region of the CAR of the present application comprises an amino acid sequence having one or more amino acid additions, deletions, mutations as compared to SEQ ID No. 18. In some embodiments, the hinge region of the CAR of the present application comprises an amino acid sequence having 1, 2, 3, 4 or 5 amino acid additions, deletions, mutations as compared to SEQ ID No. 18.

### Transmembrane Domain

Transmembrane domain consists of a hydrophobic a helix that spans the cell membrane to anchor the CAR in the T cell surface. Some evidence has suggested that the transmembrane domain can affect CAR T cell function.

Any suitable transmembrane domain known in the art can be used in the CAR of the present application. In some embodiments, the transmembrane domain of the CAR of present application can be derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

In some embodiments, the transmembrane domain of the CAR of the present application can be a native transmembrane domain of a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2. In some embodiments, the transmembrane domain of the present application can be a modified transmembrane domain of a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

In some embodiments, the transmembrane domain can be derived from the same polypeptide as the hinge region. In some embodiments the transmembrane domain can be derived from a different polypeptide from the hinge region.

In some embodiments, the transmembrane domain of the CAR of the present application can be derived from CD8. In some embodiments, the transmembrane domain of the CAR of the present application can be the transmembrane domain of native CD8. In some embodiments, the transmembrane domain of the CAR of the present application can be a modified transmembrane domain of CD8. In some embodiments, the transmembrane domain of the CAR of the present application can comprise an extended region as compared to the native transmembrane domain of CD8. In some embodiments, the transmembrane domain of the CAR of the present application can comprise a truncated transmembrane domain as compared to the native transmembrane domain of CD8.

In some embodiments, the transmembrane domain of the CAR of the present application can be derived from CD28. In some embodiments, the transmembrane domain of the CAR of the present application can be the transmembrane domain of native CD28. In some embodiments, the transmembrane domain of the CAR of the present application can be a modified transmembrane domain of CD28. In some embodiments, the transmembrane domain of the CAR of the present application can comprise an extended region as compared to the native transmembrane domain of CD28. In some embodiments, the transmembrane domain of the CAR of the present application can comprise a truncated transmembrane domain as compared to the native transmembrane domain of CD28.

In some embodiments, the transmembrane domain of the CAR of the present application comprises an amino acid sequence having at least 75%, at least 80%, at least 85%, at least 90%, at least 95%, at least 96%, at least 97%, at least 98%, or at least 99% identity with SEQ ID No. 19. In some embodiments, the transmembrane domain of the CAR of the present application comprises an amino acid sequence of SEQ ID No. 19. In some embodiments, the transmembrane domain of the CAR of the present application comprises an amino acid sequence having one or more amino acid modifications as compared to SEQ ID No. 19. In some embodiments, the transmembrane domain of the CAR of the present application comprises an amino acid sequence having 1, 2 or 3 amino acid modifications as compared to SEQ ID No. 19. In some embodiments, the transmembrane domain of the CAR of the present application comprises an amino acid sequence having one or more amino acid additions, deletions, mutations as compared to SEQ ID No. 19. In some embodiments, the transmembrane domain of the CAR of the present application comprises an amino acid sequence having 1, 2 or 3 amino acid additions, deletions, mutations as compared to SEQ ID No. 19.

In some embodiments, the transmembrane domain of the CAR of the present application comprises an amino acid sequence having at least 75%, at least 80%, at least 85%, at least 90%, at least 95%, at least 96%, at least 97%, at least 98%, or at least 99% identity with SEQ ID No. 20. In some embodiments, the transmembrane domain of the CAR of the present application comprises an amino acid sequence of SEQ ID No. 20. In some embodiments, the transmembrane domain of the CAR of the present application comprises an amino acid sequence having one or more amino acid modifications as compared to SEQ ID No. 20. In some embodiments, the transmembrane domain of the CAR of the present application comprises an amino acid sequence having 1, 2 or 3 amino acid modifications as compared to SEQ ID No. 20. In some embodiments, the transmembrane domain of the CAR of the present application comprises an amino acid sequence having one or more amino acid additions, deletions, mutations as compared to SEQ ID No. 20. In some embodiments, the transmembrane domain of the CAR of the present application comprises an amino acid sequence having 1, 2 or 3 amino acid additions, deletions, mutations as compared to SEQ ID No. 20.

In some embodiments, the transmembrane domain of the CAR of the present application comprises an amino acid sequence having at least 75%, at least 80%, at least 85%, at least 90%, at least 95%, at least 96%, at least 97%, at least 98%, or at least 99% identity with SEQ ID No. 21. In some embodiments, the transmembrane domain of the CAR of the present application comprises an amino acid sequence of SEQ ID No. 21. In some embodiments, the transmembrane domain of the CAR of the present application comprises an amino acid sequence having one or more amino acid modifications as compared to SEQ ID No. 21. In some embodiments, the transmembrane domain of the CAR of the present application comprises an amino acid sequence having 1, 2 or 3 amino acid modifications as compared to SEQ ID No. 21. In some embodiments, the transmembrane domain of the CAR of the present application comprises an amino acid sequence having one or more amino acid additions, deletions, mutations as compared to SEQ ID No. 21. In some embodiments, the transmembrane domain of the CAR of the present application comprises an amino acid sequence having 1, 2, or 3 amino acid additions, deletions, mutations as compared to SEQ ID No. 21.

In some embodiments, the transmembrane domain of the CAR of the present application comprises an amino acid sequence having at least 75%, at least 80%, at least 85%, at least 90%, at least 95%, at least 96%, at least 97%, at least 98%, or at least 99% identity with SEQ ID No. 22. In some embodiments, the transmembrane domain of the CAR of the present application comprises an amino acid sequence of SEQ ID No. 22. In some embodiments, the transmembrane domain of the CAR of the present application comprises an amino acid sequence having one or more amino acid modifications as compared to SEQ ID No. 22. In some embodiments, the transmembrane domain of the CAR of the present application comprises an amino acid sequence having 1, 2 or 3 amino acid modifications as compared to SEQ ID No. 22. In some embodiments, the transmembrane domain of the CAR of the present application comprises an amino acid sequence having one or more amino acid additions, deletions, mutations as compared to SEQ ID No. 22. In some embodiments, the transmembrane domain of the CAR of the present application comprises an amino acid sequence having 1, 2, or 3 amino acid additions, deletions, mutations as compared to SEQ ID No. 22.

### Costimulatory Domain

Costimulatory domain is a portion of endodomains or intracellular domain of CAR. It can be derived from the intracellular signaling regions of costimulatory proteins that enhance cytokine production. Costimulatory domain is required to achieve robust CAR T cell expansion, function, persistence and antitumor activity.

In some embodiments, the CAR of the present application may comprise one or more costimulatory domains. In some embodiments, the CAR of the present application comprises at least one costimulatory domain. In some embodiments, the CAR of the present application comprises at least two costimulatory domains. In some embodiments, the CAR of the present application comprises at least three costimulatory domains. In some embodiments, the CAR of the present application comprises at least four costimulatory domains. In some embodiments, the CAR of the present application comprises at least five costimulatory domains. In some embodiments, the CAR of the present application comprises at most one costimulatory domain. In some embodiments, the CAR of the present application comprises at most two costimulatory domains. In some embodiments, the CAR of the present application comprises at most three costimulatory domains. In some embodiments, the CAR of the present application comprises at most four costimulatory domains. In some embodiments, the CAR of the present application comprises at most five costimulatory domains.

Any suitable costimulatory domain known in the art can be used in the CAR of the present application. In some embodiments, the costimulatory domain of the CAR of present application can be derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the costimulatory domain can be derived from the same polypeptide as the transmembrane domain. In some embodiments the costimulatory domain can be derived from a different polypeptide from the transmembrane domain.

In some embodiments, the costimulatory domain of the CAR of the present application can be derived from 4-1BB. In some embodiments, the costimulatory domain of the CAR of the present application can be the costimulatory domain of native 4-1BB. In some embodiments, the costimulatory domain of the CAR of the present application can be a native costimulatory domain of 4-1BB. In some embodiments, the costimulatory domain of the CAR of the present application can be a modified costimulatory domain of 4-1BB. In some embodiments, the costimulatory domain of the CAR of the present application can comprise an extended region as compared to the native costimulatory domain of 4-1BB. In some embodiments, the costimulatory domain of the CAR of the present application can comprise a truncated costimulatory domain as compared to the native costimulatory domain of 4-1BB.

In some embodiments, the costimulatory domain of the CAR of the present application can be derived from CD28. In some embodiments, the costimulatory domain of the CAR of the present application can be the costimulatory domain of native CD28. In some embodiments, the costimulatory domain of the CAR of the present application can be a native costimulatory domain of CD28. In some embodiments, the costimulatory domain of the CAR of the present application can be a modified costimulatory domain of CD28. In some embodiments, the costimulatory domain of the CAR of the present application can comprise an extended region as compared to the native costimulatory domain of CD28. In some embodiments, the costimulatory domain of the CAR of the present application can comprise a truncated costimulatory domain as compared to the native costimulatory domain of CD28.

In some embodiments, the costimulatory domain of the CAR of the present application comprises an amino acid sequence having at least 75%, at least 80%, at least 85%, at least 90%, at least 95%, at least 96%, at least 97%, at least 98%, or at least 99% identity with SEQ ID No. 23. In some embodiments, the costimulatory domain of the CAR of the present application comprises an amino acid sequence of SEQ ID No. 23. In some embodiments, the costimulatory domain of the CAR of the present application comprises an amino acid sequence having one or more amino acid modifications as compared to SEQ ID No. 23. In some embodiments, the costimulatory domain of the CAR of the present application comprises an amino acid sequence having 1, 2, 3, 4 or 5 amino acid modifications as compared to SEQ ID No. 23. In some embodiments, the costimulatory domain of the CAR of the present application comprises an amino acid sequence having one or more amino acid additions, deletions, mutations as compared to SEQ ID No. 23. In some embodiments, the costimulatory domain of the CAR of the present application comprises an amino acid sequence having 1, 2, 3, 4 or 5 amino acid additions, deletions, mutations as compared to SEQ ID No. 23.

In some embodiments, the costimulatory domain of the CAR of the present application comprises an amino acid sequence having at least 75%, at least 80%, at least 85%, at least 90%, at least 95%, at least 96%, at least 97%, at least 98%, or at least 99% identity with SEQ ID No. 24. In some embodiments, the costimulatory domain of the CAR of the present application comprises an amino acid sequence of SEQ ID No. 24. In some embodiments, the costimulatory domain of the CAR of the present application comprises an amino acid sequence having one or more amino acid modifications as compared to SEQ ID No. 24. In some embodiments, the costimulatory domain of the CAR of the present application comprises an amino acid sequence having 1, 2, 3, 4 or 5 amino acid modifications as compared to SEQ ID No. 24. In some embodiments, the costimulatory domain of the CAR of the present application comprises an amino acid sequence having one or more amino acid additions, deletions, mutations as compared to SEQ ID No. 24. In some embodiments, the costimulatory domain of the CAR of the present application comprises an amino acid sequence having 1, 2, 3, 4 or 5 amino acid additions, deletions, mutations as compared to SEQ ID No. 24.

### Intracellular Signaling Region

The intracellular signaling region of a CAR of the present application can comprise a signaling domain, or any derivative, variant, or fragment thereof, involved in immune cell signaling. The intracellular signaling region of a CAR can induce activity of an immune cell comprising the CAR.

In some cases, the intracellular signaling region can be responsible for activation of at least one normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling region” refers to a polypeptide which transduces the effector function signal and directs the cell to perform a specialized function.

Any suitable intracellular signaling regions known in the art can be used for the CAR of the present disclosure, including the cytoplasmic sequences of the TCR and co-receptors that act in concert to initiate signal transduction following antigen receptor engagement, as well as any derivative or variant of these sequences and any synthetic sequence that has the same functional capability.

In some embodiments, the intracellular signaling region comprises one or more signaling domains involved in immune cell signaling, or any derivatives, variants, or fragments thereof. An immune cell signaling domain can be involved in regulating primary activation of the TCR complex in either a stimulatory way or an inhibitory way. The intracellular signaling region may be that of a T-cell receptor (TCR) complex. The intracellular signaling region of the CAR of the present application can comprise a signaling domain of an Fcγ receptor (FcγR), an Fcε receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lek, MAPK, MHC complex, NFAT, NF-κB, PLC-γ, iC3b, C3dg, C3d, and Zap70.

In some embodiments, the signaling domain includes one or more immunoreceptor tyrosine-based activation motifs or ITAMs. A signaling domain comprising an ITAM can comprise two repeats of the amino acid sequence YXXL/I separated by 6-8 amino acids, wherein each x is independently any amino acid, producing the conserved motif YXXL/I-X₆₋₈-YXXL/I. A signaling domain comprising an ITAM can be activated, for example, by phosphorylation when the antigen binding domain is bound to an epitope. A phosphorylated ITAM can function as a docking site for other proteins, for example proteins involved in various signaling pathways.

In some embodiments, the intracellular signaling domain of the CAR comprises a modified ITAM domain, e.g., a mutated, truncated, and/or optimized ITAM domain, which has altered (e.g., increased or decreased) activity compared to the native ITAM domain. A native CD3ζ intracellular signaling domain or isoform thereof comprises three ITAMs, from the transmembrane direction, are referred as ITAM1, ITAM2, and ITAM3, respectively.

The “CD3ζ intracellular signaling domain” as used herein comprises the native CD3ζ intracellular signaling domain or isoform thereof. For example, the CD3ζ intracellular signaling domain as described herein may comprise the native CD3ζ intracellular signaling domain as illustrated in SEQ ID No. 15 and the isoform thereof as illustrated in SEQ ID No. 45. A modified CD3ζ intracellular signaling domain as described herein comprises the modified CD3ζ intracellular signaling domain as compared to the native CD3ζ intracellular signaling domain (for example, as illustrated in SEQ ID No. 15) and the modified CD3ζ intracellular signaling domain as compared to the isoform (for example, as illustrated in SEQ ID No. 45).

In some embodiments, the intracellular signaling region of the CAR of the present application comprises a native CD3ζ intracellular signaling domain, for example, as illustrated in SEQ ID No. 15, or isoform thereof, for example, as illustrated in SEQ ID No. 45. In some embodiments, the intracellular signaling region of the CAR of the present application comprises a modified CD3ζ intracellular signaling domain. In some embodiments, the intracellular signaling region of the CAR of the present application comprises an extended CD3ζ intracellular signaling domain. In some embodiments, the intracellular signaling region of the CAR of the present application comprises a truncated CD3ζ intracellular signaling domain. In some embodiments, the intracellular signaling region of the CAR of the present application comprises a variant of CD3ζ intracellular signaling domain.

In some embodiments, the variant of CD3ζ intracellular signaling domain comprises a loss of function mutation. Said loss of function mutation can be any suitable mutation known in the art that result in the CD3ζ intracellular signaling domain of the CAR having less or no function (being partially or wholly inactivated). In some embodiments, the loss of function mutation comprises a substitution, insertion, deletion or any combination thereof.

In some embodiments, the variant of CD3ζ intracellular signaling domain comprises one or more loss of function mutations. In some embodiments, the variant of CD3ζ intracellular signaling domain comprises at least one loss of function mutation. In some embodiments, the variant of CD3ζ intracellular signaling domain comprises at least two loss of function mutations. In some embodiments, the variant of CD3ζ intracellular signaling domain comprises at least three loss of function mutations. In some embodiments, the variant of CD3ζ intracellular signaling domain comprises at least four loss of function mutations. In some embodiments, the variant of CD3ζ intracellular signaling domain comprises at least five loss of function mutations. In some embodiments, the variant of CD3ζ intracellular signaling domain comprises at most one loss of function mutation. In some embodiments, the variant of CD3ζ intracellular signaling domain comprises at most two loss of function mutations. In some embodiments, the variant of CD3ζ intracellular signaling domain comprises at most three loss of function mutations. In some embodiments, the variant of CD3ζ intracellular signaling domain comprises at most four loss of function mutations. In some embodiments, the variant of CD3ζ intracellular signaling domain comprises at most five loss of function mutations.

The loss of function mutation can occur at any position in the CD3ζ intracellular signaling domain of the CAR. In some embodiments, the loss of function mutation is present in one or more ITAMs of the CD3ζ intracellular signaling domain. In some embodiments, the loss of function mutation is present in one or more ITAMs of the CD3ζ intracellular signaling domain. In some embodiments, the loss of function mutation is present in any one of ITAMs of the CD3ζ intracellular signaling domain. In some embodiments, the loss of function mutation is present in ITAM1 of the CD3ζ intracellular signaling domain. In some embodiments, the loss of function mutation is present in ITAM2 of the CD3ζ intracellular signaling domain. In some embodiments, the loss of function mutation is present in ITAM3 of the CD3ζ intracellular signaling domain. In some embodiments, the loss of function mutation is present in one or more non-ITAM regions of the CD3ζ intracellular signaling domain.

In some embodiments, the loss of function mutation is present at any position of YXXL/I-X₆₋₈-YXXL/I. In some embodiments, the loss of function mutation is present at any of the Tyrosine residue (Y) of the ITAM domain (YXXL/I-X₆₋₈-YXXL/I). In some embodiments, the loss of function mutation is present at both of the two Tyrosine residues (Y) of ITAMs. In some embodiments, the Tyrosine residue (Y) is substituted with any of the residue selected from the group consisting of Alanine (A), Arginine (R), Asparagine (N), Aspartic acid (D), Cysteine (C), Glutamine (Q), Glutamic acid (E), Glycine (G), Histidine (H), IsoLeucine (I), Leucine (L), Lysine (K), Methionine (M), Phenylalanine (F), Proline (P), Serine (S), Threonine (T), Tryptophan (W), and Valine (V). In some embodiments, the Tyrosine residue (Y) is substituted with Phenylalanine (F). In some embodiments, Tyrosine residue (Y) of ITAM1 is substituted with any of the residue selected from the group consisting of Alanine (A), Arginine (R), Asparagine (N), Aspartic acid (D), Cysteine (C), Glutamine (Q), Glutamic acid (E), Glycine (G), Histidine (H), IsoLeucine (I), Leucine (L), Lysine (K), Methionine (M), Phenylalanine (F), Proline (P), Serine (S), Threonine (T), Tryptophan (W), and Valine (V). In some embodiments, any Tyrosine residue (Y) of ITAM1 is substituted with Phenylalanine (F). In some embodiments, both of Tyrosine residues (Y) of ITAM1 are substituted with Phenylalanine (F). In some embodiments, any Tyrosine residue (Y) of ITAM2 is substituted with Phenylalanine (F). In some embodiments, both of Tyrosine residues (Y) of ITAM2 are substituted with Phenylalanine (F). In some embodiments, any Tyrosine residue (Y) of ITAM3 is substituted with Phenylalanine (F). In some embodiments, both of Tyrosine residues (Y) of ITAM3 are substituted with Phenylalanine (F).

In some embodiments, the loss of function mutation is present at Leucine residue (L) of the ITAM domain. In some embodiments, the loss of function mutation is present at both of the two Leucine residues (L) of ITAMs. In some embodiments, the Leucine residue (L) is substituted with any of the residue selected from the group consisting of Alanine (A), Arginine (R), Asparagine (N), Aspartic acid (D), Cysteine (C), Glutamine (Q), Glutamic acid (E), Glycine (G), Histidine (H), IsoLeucine (I), Lysine (K), Methionine (M), Phenylalanine (F), Proline (P), Serine (S), Threonine (T), Tryptophan (W), Tyrosine (Y) and Valine (V). In some embodiments, the loss of function mutation is present in both of the two Leucine residues (L) of ITAMs.

In some embodiments, the CAR comprises a variant of CD3ζ intracellular signaling domain and the variant comprises a loss of function insertion. In some embodiments, the variant of CD3ζ intracellular signaling domain comprises at least one loss of function insertion. In some embodiments, the variant of CD3ζ intracellular signaling domain comprises at least two loss of function insertion. In some embodiments, the variant of CD3ζ intracellular signaling domain comprises at least three loss of function insertion. In some embodiments, the variant of CD3ζ intracellular signaling domain comprises at most one loss of function insertion. In some embodiments, the variant of CD3ζ intracellular signaling domain comprises at most two loss of function insertion. In some embodiments, the variant of CD3ζ intracellular signaling domain comprises at most three loss of function insertion.

In some embodiments, the loss of function insertion is not present in the ITAMs of the CD3ζ intracellular signaling domain. In some embodiments, the loss of function insertion is present between ITAMs of the CD3ζ intracellular signaling domain. In some embodiments, the loss of function insertion is present between ITAM1 and ITAM2 of the CD3ζ intracellular signaling domain. In some embodiments, the loss of function insertion is present between ITAM2 and ITAM3 of the CD3ζ intracellular signaling domain.

The loss of function insertion can be any suitable sequences capable of reduce or eliminate the functions of CD3ζ intracellular signaling domain. In some embodiments, the loss of function insertion comprises the sequence of SEQ ID No. 43 (referred as M1 herein), or a fragment thereof. In some embodiments, the loss of function insertion comprises the sequence of SEQ ID No. 44 (referred as M2 herein), or a fragment thereof.

In some embodiments, the CAR comprises a truncated CD3ζ intracellular signaling domain, and the truncated CD3ζ intracellular signaling domain is devoid of one or more ITAMs, or a fragment thereof. In some embodiments, the truncated CD3ζ intracellular signaling domain is devoid of at least one ITAM of CD3ζ intracellular signaling domain, or a fragment thereof. In some embodiments, the truncated CD3ζ intracellular signaling domain is devoid of at least two ITAM of CD3ζ intracellular signaling domain, or a fragment thereof. In some embodiments, the truncated CD3ζ intracellular signaling domain is devoid of at most one ITAM of CD3ζ intracellular signaling domain, or a fragment thereof. In some embodiments, the truncated CD3ζ intracellular signaling domain is devoid of at most two ITAM of CD3ζ intracellular signaling domain, or a fragment thereof. In some embodiments, the truncated CD3ζ intracellular signaling domain is devoid of ITAM1, or a fragment thereof. In some embodiments, the truncated CD3ζ intracellular signaling domain is devoid of ITAM2, or a fragment thereof. In some embodiments, the truncated CD3ζ intracellular signaling domain is devoid of ITAM3 or a fragment thereof. In some embodiments, the truncated CD3ζ intracellular signaling domain is devoid of ITAM1 and ITAM2, or a fragment thereof. In some embodiments, the truncated CD3ζ intracellular signaling domain is devoid of ITAM1 and ITAM3, or a fragment thereof. In some embodiments, the truncated CD3ζ intracellular signaling domain is devoid of ITAM2 and ITAM3, or a fragment thereof.

In some embodiments, the truncated CD3ζ intracellular signaling domain is devoid of an amino acid sequence of SEQ ID NO.1 or a fragment thereof. In some embodiments, the truncated CD3ζ intracellular signaling domain is devoid of an amino acid sequence of SEQ ID NO.2 or a fragment thereof. In some embodiments, the truncated CD3ζ intracellular signaling domain is devoid of an amino acid sequence of SEQ ID NO.3 or a fragment thereof. In some embodiments, the truncated CD3ζ intracellular signaling domain is devoid of an amino acid sequence of SEQ ID NO. 4 or a fragment thereof. In some embodiments, the truncated CD3ζ intracellular signaling domain is devoid of an amino acid sequence of SEQ ID NO.5 or a fragment thereof. In some embodiments, the truncated CD3ζ intracellular signaling domain is devoid of an amino acid sequence of SEQ ID NO.6 or a fragment thereof. In some embodiments, the truncated CD3ζ intracellular signaling domain is devoid of an amino acid sequence of SEQ ID NO.7 or a fragment thereof.

In some embodiments, the intracellular signaling region of the CAR of the present application comprises three ITAMs, or a fragment thereof. In some embodiments, the intracellular signaling region of the CAR of the present application comprises two ITAMs, or a fragment thereof. In some embodiments, the intracellular signaling region of the CAR of the present application comprises ITAM2 and ITAM3, or a fragment thereof. In some embodiments, the intracellular signaling region of the CAR of the present application comprises one ITAM, or a fragment thereof.

In some embodiments, the intracellular signaling region of the CAR of the present application comprises three ITAM1, ITAM2 and ITAM3, or a fragment thereof. In some embodiments, the intracellular signaling region of the CAR of the present application comprises ITAM1 and ITAM2, or a fragment thereof. In some embodiments, the intracellular signaling region of the CAR of the present application comprises ITAM1 and ITAM3, or a fragment thereof. In some embodiments, the intracellular signaling region of the CAR of the present application comprises ITAM1, or a fragment thereof. In some embodiments, the intracellular signaling region of the CAR of the present application comprises ITAM2, or a fragment thereof. In some embodiments, the intracellular signaling region of the CAR of the present application comprises ITAM3, or a fragment thereof.

In some embodiments, the intracellular signaling region of the CAR of the present application comprises at most three ITAMs, or a fragment thereof. In some embodiments, the intracellular signaling region of the CAR of the present application comprises at most two ITAMs, or a fragment thereof. In some embodiments, the intracellular signaling region of the CAR of the present application comprises at most one ITAM, or a fragment thereof. In some embodiments, the intracellular signaling region of the CAR of the present application comprises at least three ITAMs, or a fragment thereof. In some embodiments, the intracellular signaling region of the CAR of the present application comprises at least two ITAMs, or a fragment thereof. In some embodiments, the intracellular signaling region of the CAR of the present application comprises at least one ITAM, or a fragment thereof.

In some embodiments, the intracellular signaling region of the CAR of the present application comprises an amino acid sequence of SEQ ID NO. 8. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 8. In some embodiments, the intracellular signaling region of the CAR of the present application comprises an amino acid sequence having one or more amino acid modifications as compared to SEQ ID No. 8. In some embodiments, the intracellular signaling region of the CAR of the present application comprises an amino acid sequence having 1, 2, 3 4 or 5 amino acid modifications as compared to SEQ ID No. 8. In some embodiments, the intracellular signaling region of the CAR of the present application comprises an amino acid sequence having one or more amino acid additions, deletions, mutations as compared to SEQ ID No. 8. In some embodiments, the intracellular signaling region of the CAR of the present application comprises an amino acid sequence having 1, 2, 3, 4, or 5 amino acid additions, deletions, mutations as compared to SEQ ID No. 8.

In some embodiments, the intracellular signaling region of the CAR of the present application comprises an amino acid sequence of SEQ ID NO. 9. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 9. In some embodiments, the intracellular signaling region of the CAR of the present application comprises an amino acid sequence having one or more amino acid modifications as compared to SEQ ID No. 9. In some embodiments, the intracellular signaling region of the CAR of the present application comprises an amino acid sequence having 1, 2, 3, 4, or 5 amino acid modifications as compared to SEQ ID No. 9. In some embodiments, the intracellular signaling region of the CAR of the present application comprises an amino acid sequence having one or more amino acid additions, deletions, mutations as compared to SEQ ID No. 9. In some embodiments, the intracellular signaling region of the CAR of the present application comprises an amino acid sequence having 1, 2, 3, 4, or 5 amino acid additions, deletions, mutations as compared to SEQ ID No. 9.

In some embodiments, the intracellular signaling region of the CAR of the present application comprises an amino acid sequence of SEQ ID NO. 10. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 10. In some embodiments, the intracellular signaling region of the CAR of the present application comprises an amino acid sequence having one or more amino acid modifications as compared to SEQ ID No. 10. In some embodiments, the intracellular signaling region of the CAR of the present application comprises an amino acid sequence having 1, 2, 3, 4 or 5 amino acid modifications as compared to SEQ ID No. 10. In some embodiments, the intracellular signaling region of the CAR of the present application comprises an amino acid sequence having one or more amino acid additions, deletions, mutations as compared to SEQ ID No. 10. In some embodiments, the intracellular signaling region of the CAR of the present application comprises an amino acid sequence having 1, 2, 3, 4 or 5 amino acid additions, deletions, mutations as compared to SEQ ID No. 10.

In some embodiments, the intracellular signaling region of the CAR of the present application comprises an amino acid sequence of SEQ ID NO. 11. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 11. In some embodiments, the intracellular signaling region of the CAR of the present application comprises an amino acid sequence having one or more amino acid modifications as compared to SEQ ID No. 11. In some embodiments, the intracellular signaling region of the CAR of the present application comprises an amino acid sequence having 1, 2, 3, 4 or 5 amino acid modifications as compared to SEQ ID No. 11. In some embodiments, the intracellular signaling region of the CAR of the present application comprises an amino acid sequence having one or more amino acid additions, deletions, mutations as compared to SEQ ID No. 11. In some embodiments, the intracellular signaling region of the CAR of the present application comprises an amino acid sequence having 1, 2, 3, 4 or 5 amino acid additions, deletions, mutations as compared to SEQ ID No. 11.

In some embodiments, the intracellular signaling region of the CAR of the present application comprises an amino acid sequence of SEQ ID NO. 12. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 12. In some embodiments, the intracellular signaling region of the CAR of the present application comprises an amino acid sequence having one or more amino acid modifications as compared to SEQ ID No. 12. In some embodiments, the intracellular signaling region of the CAR of the present application comprises an amino acid sequence having 1, 2, 3, 4 or 5 amino acid modifications as compared to SEQ ID No. 12. In some embodiments, the intracellular signaling region of the CAR of the present application comprises an amino acid sequence having one or more amino acid additions, deletions, mutations as compared to SEQ ID No. 12. In some embodiments, the intracellular signaling region of the CAR of the present application comprises an amino acid sequence having 1, 2, 3, 4 or 5 amino acid additions, deletions, mutations as compared to SEQ ID No. 12.

In some embodiments, the intracellular signaling region of the CAR of the present application comprises an amino acid sequence of SEQ ID NO. 13. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 13. In some embodiments, the intracellular signaling region of the CAR of the present application comprises an amino acid sequence having one or more amino acid modifications as compared to SEQ ID No. 13. In some embodiments, the intracellular signaling region of the CAR of the present application comprises an amino acid sequence having 1, 2, 3, 4 or 5 amino acid modifications as compared to SEQ ID No. 13. In some embodiments, the intracellular signaling region of the CAR of the present application comprises an amino acid sequence having one or more amino acid additions, deletions, mutations as compared to SEQ ID No. 13. In some embodiments, the intracellular signaling region of the CAR of the present application comprises an amino acid sequence having 1, 2, 3, 4 or 5 amino acid additions, deletions, mutations as compared to SEQ ID No. 13.

In some embodiments, the intracellular signaling region of the CAR of the present application comprises an amino acid sequence of SEQ ID NO. 14. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 14. In some embodiments, the intracellular signaling region of the CAR of the present application comprises an amino acid sequence having one or more amino acid modifications as compared to SEQ ID No. 14. In some embodiments, the intracellular signaling region of the CAR of the present application comprises an amino acid sequence having 1, 2, 3, 4 or 5 amino acid modifications as compared to SEQ ID No. 14. In some embodiments, the intracellular signaling region of the CAR of the present application comprises an amino acid sequence having one or more amino acid additions, deletions, mutations as compared to SEQ ID No. 14. In some embodiments, the intracellular signaling region of the CAR of the present application comprises an amino acid sequence having 1, 2, 3, 4 or 5 amino acid additions, deletions, mutations as compared to SEQ ID No. 14.

In some embodiments, the intracellular signaling region of the CAR of the present application comprises an amino acid sequence of SEQ ID NO. 15. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 15. In some embodiments, the intracellular signaling region of the CAR of the present application comprises an amino acid sequence having one or more amino acid modifications as compared to SEQ ID No. 15. In some embodiments, the intracellular signaling region of the CAR of the present application comprises an amino acid sequence having 1, 2, 3, 4 or 5 amino acid modifications as compared to SEQ ID No. 15. In some embodiments, the intracellular signaling region of the CAR of the present application comprises an amino acid sequence having one or more amino acid additions, deletions, mutations as compared to SEQ ID No. 15. In some embodiments, the intracellular signaling region of the CAR of the present application comprises an amino acid sequence having 1, 2, 3, 4 or 5 amino acid additions, deletions, mutations as compared to SEQ ID No. 15.

In some embodiments, the intracellular signaling region of the CAR of the present application comprises an amino acid sequence of SEQ ID NO. 45. In some embodiments, the intracellular signaling region of the CAR of the present application comprises an amino acid sequence having at least 75%, at least 80%, at least 85%, at least 90%, at least 95%, at least 96%, at least 97%, at least 98%, or at least 99% identity with SEQ ID NO. 45. In some embodiments, the intracellular signaling region of the CAR of the present application comprises an amino acid sequence having one or more amino acid modifications as compared to SEQ ID No. 45. In some embodiments, the intracellular signaling region of the CAR of the present application comprises an amino acid sequence having 1, 2, 3, 4 or 5 amino acid modifications as compared to SEQ ID No. 45. In some embodiments, the intracellular signaling region of the CAR of the present application comprises an amino acid sequence having one or more amino acid additions, deletions, mutations as compared to SEQ ID No. 45. In some embodiments, the intracellular signaling region of the CAR of the present application comprises an amino acid sequence having 1, 2, 3, 4 or 5 amino acid additions, deletions, mutations as compared to SEQ ID No. 45.

### Antigen Binding Domain

A CAR provided herein can comprise one or more antigen binding domains. In some cases, a CAR provided herein comprises an antigen binding domain that can target both an immune cell antigen (e.g., to inhibit killing activity of a T cell or NK cell) and a disease-associated antigen (e.g., a tumor-associated antigen). For example, an antigen binding domain targeting both immune cell antigens and cancer antigens include, but not limited to, CD2, CD3, CD4, CD5, CD7, CD8, CD30, CD38, CD45, CD48, CD50, CD52, CD56, CD69, CD100, CD122, CD132, CD137, CD161, CD159a, CD159c, CD279, CD314, CD319 (CS1) and TCR.

In some cases, a CAR provided herein comprises a single antigen binding domain. In some embodiments, the single antigen binding domain is a scFv or sdAb. In some cases, the antigen can be any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2. In some embodiments, the antigen is CD7.

In some cases, a CAR provided herein comprises two antigen binding domains such that one individual CAR is a bispecific CAR, targeting two different antigens. In some cases, the antigen binding domain may be selected from any antigen binding domain disclosed herein. In some cases, one of the antigen binding domains may be a scFv, and the other antigen binding domains may be a single domain antibody (sdAb). In some cases, both antigen binding domains are scFv. In some cases, both antigen binding domains are sdAb.

In some cases, the first antigen binding domain of the bispecific CAR targets an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2.

In some cases, the second antigen binding domain of the bispecific CAR targets an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2.

The two antigen binding domains of a bispecific CAR can have a tandem structure, a parallel structure or a loop structure.

In some cases, a CAR comprises two antigen binding domains arranged in a tandem form. In some embodiments, the first antigen binding domain or the second antigen binding domain is a scFv. In some embodiments, the first antigen binding domain and the second antigen binding domain is arranged, from amino terminus to carboxyl terminus, in any of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain.

In some cases, a CAR comprising two antigen binding domains arranged in a loop form. In some cases, the first antigen binding domain and the second antigen binding domain is arranged, from amino terminus to carboxyl terminus, in any of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; and (viii) VH1-VH2-VL2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain.

In some cases, a CAR comprising two antigen binding domains are arranged in a parallel form. The parallel form can comprise a full construct of a first CAR having a first antigen binding domain linked to a full construct of a second CAR having a second antigen binding domain.

In some cases, a CAR provided herein is a multivalent CAR such as a tri-specific CAR (tri-CAR) comprising three antigen binding domains. In some cases, the first antigen binding domain of the tri-specific CAR targets an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2. In some embodiments, the first antigen binding domain targets CS1, CD7, CD137, BCMA or CD19.

In some cases, the second antigen binding domain of the tri-specific CAR targets an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2. In some embodiments, the second antigen binding domain targets CS1, CD7, CD137, BCMA or CD19.

In some cases, the third antigen binding domain of the tri-specific CAR targets an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2. In some embodiments, the third antigen binding domain targets CS1, CD7, CD137, BCMA or CD19.

In some embodiments, provided is the nucleic acid comprising a first sequence encoding one or more chimeric antigen receptors (CARs) as described above, wherein the CAR can comprise a monovalent or multivalent (such as bivalent) antigen binding domain and wherein each CAR of the one or more CARs can further comprise a transmembrane domain and an intracellular signaling region as described above.

The antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2.

In some embodiments, the nucleic acid can comprise a first sequence encoding one or more chimeric antigen receptor (CAR), wherein the CAR can comprise (i) a first antigen binding domain linked to (ii) a second antigen binding domain, and wherein each CAR of the one or more CARs can further comprise a transmembrane domain and an intracellular signaling region as described above.

The first antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2.

In some cases, the second antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, and TROP-2.

In some embodiments, the nucleic acid can comprise a first sequence encoding one or more chimeric antigen receptor (CAR), wherein the CAR can comprise (i) a first antigen binding domain linked to (ii) a second antigen binding domain, (iii) a third antigen binding domain, and wherein each CAR of the one or more CARs can further comprise a transmembrane domain and an intracellular signaling region as described above.

The first antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, and TROP-2.

In some cases, the second antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, and TROP-2.

In some cases, the third antigen binding domain can target an antigen of any one selected the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRa, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, and TROP-2.

The nucleic acid molecule can further comprise a second sequence encoding an enhancer moiety, which enhancer moiety can enhance one or more activities of the CAR when expressed in a cell. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof. The nucleic acid molecule can further comprise a second sequence encoding an inducible cell death moiety, which inducible cell death moiety, when expressed in a cell, can effect death of the cell upon contacting the inducible cell death moiety with a cell death activator. The inducible cell death moiety can be selected from the group consisting of rapaCasp9, iCasp9, HSV-TK, ΔCD20, mTMPK, ΔCD19, RQR8, and EGFRt.

The nuclei acid molecule can further comprise a third sequence flanked by the first sequence and the second sequence, wherein the third sequence can encode a cleavable linker. The cleavable linker can be a self-cleaving peptide. In some embodiments, the cleavable linker is P2A linker.

The nucleic acid molecule can further comprise a regulatory sequence regulating expression of the first sequence and/or the second sequence.

Also contemplated in the present disclosure is a kit comprising the nucleic acid molecule described herein.

In some cases, the nucleic acid encoding the CAR described herein can be delivered into an immune cell for expression of the CAR to generate an engineered cell.

### Source Cell

The present disclosure provides an engineered cell, such as an engineered immune cell. The engineered immune cell can be prepared from a cell (e.g., an immune cell) isolated from a sample obtained from a subject. The engineered immune cell can be prepared from a cell line cell. The immune cell used to prepare the engineered immune cell can be a T cell, a B cell, a natural killer (NK) cell or a macrophage. The immune cell used to prepare the engineered immune cell can be an innate lymphocyte (ILC).

The immune cell used to prepare the engineered immune cell can be a stem cell. The stem cell can be a hematopoietic stem cell (HSC) or an induced pluripotent stem cell (iPSC).

The immune cell may comprise a T-cell receptor (TCR). The TCR can be endogenous TCR of the immune cell. In some cases, the endogenous TCR can be inactivated. For example, a gene encoding a subunit of the TCR can be inactivated. For example, the immune cell can be an alpha beta T cells with impaired TCRs such that the immune cells can avoid GVHD. For another example, the function of the endogenous TCR can be inhibited by an inhibitor such as TCR-derived peptides, peptides derived from amino acid sequences of fusion and other protein regions of various viruses, antibodies and small molecule inhibitors. The viruses from which the TCR inhibiting peptides can be derived from include, but are not limited to, severe acute respiratory syndrome coronavirus (SARS-CoV), herpesvirus saimiri (HVS), human herpesvirus 6 (HHV-6), Lassa virus (LASV), lymphocytic choriomeningitis virus (LCMV), Mopeia virus (MOPV), Tacaribe virus (TACV), Friend murine leukemia virus (MLV); human T lymphotropic virus type 1 (HTLV-1,); herpesvirus ateles (HVA); Marburg virus (MARV); Sudan Ebola virus (SEBOV); and Zaire Ebola virus (ZEBOV).

In some cases, the immune cells can be T cells containing TCRs that may not cause GVHD responses. For example, the immune cell can be an alpha beta T cell with TCRs that can recognize specific antigens such as viral specific antigen, tumor-associated antigens (TAAs) or tumor-specific antigens (TSAs). For another example, the immune cell can be a gamma delta T cell or a natural killer T (NKT) cell. For another example, the immune cell can be induced pluripotent stem cells produced from antigen-specific T cells (e.g., antigen-specific cytotoxic T cells). The immune cell can be cord-blood T cells.

The immune cell may comprise a cell surface marker. The cell surface marker can be an immune cell antigen. The gene encoding the immune cell antigen of the immune cell used for preparing the engineered immune cell can be inactivated. Examples of immune cell antigens include, but are not limited to, CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ and SLAMF7. For example, in some cases, the gene encoding CD7 of the immune cell is inactivated.

The immune cells can be isolated from a sample from a subject. The subject can be a healthy donor. The subject can have a condition (e.g., a disease such as cancer). The sample can be a bodily fluid or a tissue, including but not limited to, peripheral blood mononuclear cells, bone marrow, lymph node tissue, cord blood, thymus tissue, tissue from a site of infection, ascites, pleural effusion, spleen tissue, and tumors. In some cases, a sample comprises NK cells, NKT cells, T-cells or T-cell progenitor cells. For example, in some cases, the sample is an umbilical cord blood sample, a peripheral blood sample (e.g., a mononuclear cell fraction) or a sample from the subject comprising pluripotent cells. In some embodiments, a sample from the subject can be cultured to generate induced pluripotent stem (iPS) cells and these cells used to produce NK cells, NKT cells or T-cells. Cell samples may be cultured directly from the subject or may be cryopreserved prior to use. In some embodiments, obtaining a cell sample comprises collecting a cell sample. In other aspects, the sample is obtained by a third party. In still further aspects, a sample from a subject can be treated to purify or enrich the T-cells or T-cell progenitors in the sample. For example, the sample can be subjected to gradient purification, cell culture selection and/or cell sorting (e.g., via fluorescence-activated cell sorting (FACS)).

The immune cell can be an NK cell. The NK cells can be obtained from peripheral blood, cord-blood, or other sources described herein. The NK cells can be derived from induced pluripotent stem cells.

In some embodiments, a cell that can be utilized in a method provided herein can be positive or negative for a given factor. In some embodiments, a cell utilized in a method provided herein can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b-cell, a BDCA1+ cell, a BDCA1-cell, an L-selectin+ cell, an L-selectin-cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15-cell, a lectin-like receptor Gi positive cell, a lectin-like receptor Gi negative cell, or an differentiated or de-differentiated cell thereof. The examples of factors expressed by cells is not intended to be limiting, and a person having skill in the art will appreciate that a cell may be positive or negative for any factor known in the art. In some embodiments, a cell may be positive for two or more factors. For example, a cell may be CD4+ and CD8+. In some embodiments, a cell may be negative for two or more factors. For example, a cell may be CD25−, CD44−, and CD69−. In some embodiments, a cell may be positive for one or more factors, and negative for one or more factors. For example, a cell may be CD4+ and CD8−. In some embodiments, a cellular marker provided herein can be utilized to select, enrich, or deplete a population of cells. In some embodiments, enriching comprises selecting a monocyte fraction. In some embodiments, enriching comprises sorting a population of immune cells from a monocyte fraction. In some embodiments, the cells may be selected for having or not having one or more given factors (e.g., cells may be separated based on the presence or absence of one or more factors). In some embodiments, the selected cells can also be transduced and/or expanded in vitro. The selected cells can be expanded in vitro prior to infusion. In some embodiments, selected cells can be transduced with a vector provided herein. It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells. In some cases, a cell can be a stem memory TSCM cell comprised of CD45RO (−), CCR7(+), CD45RA (+), CD62L+(L-selectin), CD27+, CD28+ and IL-7Rα+, stem memory cells can also express CD95, IL-2R3, CXCR3, and LFA-1, and show numerous functional attributes distinctive of stem memory cells. Cells provided herein can also be central memory TCM cells comprising L-selectin and CCR7, where the central memory cells can secrete, for example, IL-2, but not IFNγ or IL-4. Cells can also be effector memory TEM cells comprising L-selectin or CCR7 and produce, for example, effector cytokines such as IFNγ and IL-4. In some cases, a population of cells can be introduced to a subject. For example, a population of cells can be a combination of T cells and NK cells. In other cases, a population can be a combination of naïve cells and effector cells. A population of cells can be TILs.

The source immune cells can be T cells. The T cells can be alpha beta T cells or gamma delta T cells. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, various T cell lines may be used. In certain embodiments of the present disclosure, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments of the present disclosure, the cells are washed with phosphate buffered saline (PBS). In an alternative embodiment, the wash solution lacks calcium and may lack magnesium or may lack many if not all divalent cations. Initial activation steps in the absence of calcium may lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types. Further, use of longer incubation times can increase the efficiency of capture of CD8+T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. In some cases, multiple rounds of selection can also be used. In certain embodiments, it may be useful to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. An example method can be cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+T cells that normally have weaker CD28 expression.

In a related embodiment, lower concentrations of cells may be used. By significantly diluting the mixture of T cells and surface (e.g., particles such as beads), interactions between the particles and cells is minimized. This method can select for cells that express high amounts of desired antigens to be bound to the particles. For example, CD4+T cells express higher levels of CD28 and are more efficiently captured than CD8+T cells in dilute concentrations. In some embodiments, the concentration of cells used is 5×10⁶/ml. In other embodiments, the concentration used can be from about 1×10⁵/ml to 1×10⁶/ml, and any integer value in between. In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present disclosure.

In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

### Engineered Immune Cell

The engineered immune cell provided herein can exhibit enhanced activity toward tumor cells, but with reduced side effects such as cytokine release syndrome (CRS), GVHD, and/or host rejection of a graft (HVG). The engineered immune cell can target a disease-associated antigen (e.g., tumor-associated antigen, or tumor cell marker) and at the same time suppress host immune cells. One or more endogenous genes (e.g., a gene encoding a subunit of a TCR, a gene encoding a subunit of an MHC molecule, or a gene encoding a cell surface marker) of the engineered immune cell can be inactivated.

In some cases, the engineered immune cell comprises a single CAR. In some cases, the engineered immune cell comprises a first CAR and a second CAR, each targeting a different antigen. In some cases, the engineered immune cell comprises a CAR having a first antigen binding domain and a second antigen binding domain. In some cases, the engineered immune cell comprises a first CAR, a second CAR and a third CAR, each targeting a different antigen. In some cases, the engineered immune cell comprises a CAR having a first antigen binding domain, a second antigen binding domain and a third antigen binding domain.

In some cases, the endogenous antigen of the engineered immune cell can be inactivated in the engineered immune cell. In some cases, a gene encoding the endogenous antigen can be inactivated (e.g., silenced or knocked out) in the engineered immune cell. In some cases, a gene encoding endogenous CD7 can be inactivated (e.g., silenced or knocked out) in the engineered immune cell.

In some cases, the endogenous T cell receptor (TCR) of the engineered immune cell can be inactivated. In some cases, a gene encoding a subunit of the endogenous TCR of the engineered immune cell can be inactivated such that the endogenous TCR can be inactivated. The gene encoding the subunit can be TCRα, TCRβ, CD3ε, CD3δ, CD3γ, or CD3ζ.

In some cases, the endogenous MHC molecule of the engineered immune cell can be inactivated. In some cases, the endogenous MHC molecule comprises MHC class I molecule and MHC class II molecule. In some cases, a gene encoding MHC I molecule can be inactivated. The gene encoding MHC I molecule includes but is not limited to HLA-A, HLA-B, HLA-C, HLA-E, HLA-F and HLA-G. In some embodiments, the expression of one or more endogenous HLA genes of the engineered immune cell may be knocked out or partially knocked out. For example, any one of more of HLA-A, HLA-B, HLA-C, HLA-E, HLA-F and HLA-G of the engineered immune cell may be knocked out or partially knocked out. In some cases, an endogenous HLA-A, HLA-B, HLA-C, HLA-E, HLA-F or HLA-G can be knocked out or partially knocked out to reduce T cell killing activity. In some cases, any one of more of HLA-A, HLA-B, HLA-C, HLA-E, HLA-F and HLA-G of the engineered immune cell can remain intact. In some cases, a subunit of the endogenous MHC molecule in said engineered immune cell can be inactivated such that the endogenous MHC molecule is inactive. In some cases, B2M subunit of the endogenous MHC molecule in said engineered immune cell is inactivated. In some cases, B2M subunit of the endogenous MHC molecule in said engineered immune cell is knocked out or partially knocked out.

In some cases, a killer/phagocyte inhibitor of the engineered immune cell can be overexpressed. In some other cases, an endogenous HLA can be knocked out with co-expression of killer/phagocyte inhibitor(s). For example, B2M can be knocked out with co-expression of killer/phagocyte inhibitors. The killer/phagocyte inhibitor may suppress immune response toward the engineered immune cell. The killer/phagocyte inhibitors include, but are not limited to, HLA-E single chain trimer, HLA-G, CD47, CD24, FASL, PDL1, or functional domains thereof.

In some cases, the engineered immune cell can exhibit (i) enhanced degree of persistence by remaining viable in vitro while in presence of cells that are heterologous to the engineered immune cell, including but not limited to heterologous T cells, heterologous NK cells and the mixture of the heterologous T cells and heterologous NK cells, (ii) enhanced degree of expansion, or (iii) enhanced cytotoxicity against a target cell comprising the antigen, compared to an additional engineered immune cell comprising the one or more CARs without the inactivation of the TCR, MHC molecule and/or immune cell antigen. In some cases, the engineered immune cell can be characterized by exhibiting two or more of (i) enhanced degree of persistence by remaining viable in vitro while in presence of cells that are heterologous to the engineered immune cell, including but not limited to heterologous T cells, heterologous NK cells and the mixture of the heterologous T cells and heterologous NK cells, (ii) enhanced degree of expansion, and (iii) enhanced cytotoxicity.

The binding moiety can comprise a first antigen binding domain capable of binding to an immune cell antigen and a second antigen binding domain capable of binding to a disease-associated antigen. Each CAR of the one or more CARs may further comprise a hinge, a transmembrane domain, a costimulatory and an intracellular signaling region as described above.

The engineered immune cell can also comprise an enhancer moiety capable of enhancing one or more activities of the engineered immune cell.

In some cases, the endogenous immune cell antigen of the engineered immune cell be inactivated. In some cases, the endogenous T cell receptor (TCR) of the engineered immune cell can be inactivated. In some cases, the endogenous MHC molecule of the engineered immune cell can be inactivated. In some cases, any two or more of the endogenous immune cell antigens, the endogenous TCR, and the endogenous MHC molecule of the engineered immune cell can be inactivated. In some cases, the endogenous immune cell antigen, the endogenous TCR, and the endogenous MHC molecule of the engineered immune cell can be all inactivated.

In some cases, the engineered immune cell can exhibit (i) enhanced degree of persistence by remaining viable in vitro while in presence of cells that are heterologous to the engineered immune cell, including but not limited to heterologous T cells, heterologous NK cells and the mixture of the heterologous T cells and heterologous NK cells, (ii) enhanced degree of expansion, or (iii) enhanced cytotoxicity against a target cell comprising the immune cell antigen or the disease-associated antigen, compared to an additional engineered immune cell comprising the one or more CARs without the inactivation of the TCR, MHC molecule and/or immune cell antigen. In some cases, the engineered immune cell can be characterized by exhibiting two or more of (i) enhanced degree of persistence by remaining viable in vitro while in presence of cells that are heterologous to the engineered immune cell, including but not limited to heterologous T cells, heterologous NK cells and the mixture of the heterologous T cells and heterologous NK cells, (ii) enhanced degree of expansion, and (iii) enhanced cytotoxicity.

The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be self-activating through self-oligomerizing. The enhancer moiety can be self-activating through self-dimerizing.

The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The engineered immune cell can further comprise an inducible cell death moiety, which can effect suicide of the engineered immune cell upon contact with a cell death activator. The inducible cell death moiety can be selected from the group consisting of rapaCasp9, iCasp9, HSV-TK, ΔCD20, mTMPK, ΔCD19, RQR8, and EGFRt. In some cases, the inducible cell death moiety is EGFRt, and the cell death activator is an antibody or an antigen binding fragment thereof that binds EGFRt. In some cases, the inducible cell death moiety is HSV-TK, and the cell death activator is GCV. In some cases, the inducible cell death moiety is iCasp9, and the cell death activator is AP1903. The cell death activator can comprise a nucleic acid, a polynucleotide, an amino acid, a polypeptide, lipid, a carbohydrate, a small molecule, an enzyme, a ribosome, a proteasome, a variant thereof, or any combination thereof.

The engineered immune cell provided herein can comprise a chimeric polypeptide comprising (i) an enhancer moiety capable of enhancing one or more activities of the engineered immune cell, and (ii) an inducible cell death moiety capable of effecting death of the engineered immune cell upon contacting the chimeric polypeptide with a cell death activator, wherein the enhancer moiety is linked to the inducible cell death moiety. In some cases, the enhancer moiety and the inducible moiety may be linked by a linker. The linker can be a cleavable linker, for example, a self-cleaving peptide.

The engineered immune cell can further comprise one or more chimeric polypeptide receptors (CPRs) comprising a binding moiety, wherein the binding moiety comprises (i) a first antigen binding domain, which first antigen binding domain suppresses or reduces a subject's immune response toward the engineered immune cell when administered into the subject and (ii) a second antigen binding domain capable of binding to a disease-associated antigen. An individual CPR of the one or more CPRs can comprise (i) the first antigen binding domain, (ii) the second antigen binding domain, or (iii) both the first antigen binding domain and the second antigen binding domain. A CPR of the one or more CPRs can further comprise a transmembrane domain and an intracellular signaling region. In some cases, the one or more CPRs in the engineered immune cell are one or more chimeric antigen receptors (CARs) or engineered T cell receptors (TCRs). In some cases, the engineered immune cells comprise both CARs and engineered TCRs.

The engineered TCR can be a TCR fusion protein. For example, the TCR fusion protein can comprise a heterologous antigen binding domain fused to one or more subunits of a TCR complex. In some cases, the TCR fusion protein can comprise a TCR subunit comprising at least a portion of a TCR extracellular domain and a TCR intracellular domain; and an antibody domain comprising an antigen binding domain, where the TCR subunit and the antibody domain are linked. The TCR fusion protein can incorporate into a TCR complex when expressed in a T cell. In some cases, the TCR fusion protein can further comprise a TCR transmembrane domain. The TCR extracellular domain, the TCR intracellular domain, or the TCR transmembrane domain can be derived from TCR alpha chain, TCR beta chain, TCR gamma chain, TCR delta chain, CD3 epsilon, CD3 gamma, CD3 delta or CD3 zeta. In some cases, an endogenous TCR of the engineered immune cell comprising an engineered TCR is inactivated. In some cases, the engineered immune cell comprising inactivated endogenous TCR may not cause GVHD. For example, a gene encoding an endogenous TCR subunit can be inactivated. For another example, a gene encoding an endogenous TCR subunit may be mutated such that an endogenous TCR may not be formed.

The engineered immune cell provided herein can comprise a chimeric polypeptide comprising (i) an enhancer moiety capable of enhancing one or more activities of the engineered immune cell, and (ii) an inducible cell death moiety capable of effecting death of the engineered immune cell upon contacting the chimeric polypeptide with a cell death activator. In some cases, the enhancer moiety is linked to the inducible cell death moiety. The one or more chimeric antigen receptors (CARs) can comprise a binding moiety. The binding moiety can comprise (i) a first antigen binding domain, which first antigen binding domain suppresses or reduces a subject's immune response toward the engineered immune cell when administered into the subject and (ii) a second antigen binding domain capable of binding to a disease-associated antigen.

In some cases, an individual CAR of the one or more CARs comprises (i) the first antigen binding domain or (ii) the second antigen binding domain. In some cases, an individual CAR of the one or more CARs comprises both the first antigen binding domain and the second antigen binding domain. In some cases, each CAR of the one or more CARs further comprises a hinge domain, transmembrane domain and an intracellular signaling region as described above.

In some cases, the endogenous T cell receptor (TCR) of the engineered immune cell can be inactivated. In some cases, a gene encoding a subunit of the endogenous TCR of the engineered immune cell can be inactivated such that the endogenous TCR is inactivated. The gene encoding the subunit can be TCRα, TCRβ, CD3ε, CD3δ, CD3γ, or CD3ζ.

In some cases, the endogenous MHC molecule of the engineered immune cell can be inactivated. In some cases, the endogenous MHC molecule comprises MHC class I molecule and MHC class II molecule. In some cases, a gene encoding MHC I molecule is inactivated. The gene encoding MHC I molecule comprises but is not limited to HLA-A, HLA-B, HLA-C, HLA-E, HLA-F and HLA-G. In some embodiments, the expression of one or more endogenous HLA genes of the engineered immune cell may be knocked out or partially knocked out. For example, any one of more of HLA-A, HLA-B, HLA-C, HLA-E, HLA-F and HLA-G of the engineered immune cell may be knocked out or partially knocked out. In some cases, an endogenous HLA-A, HLA-B, HLA-C, HLA-E, HLA-F or HLA-G can be knocked out or partially knocked out to reduce T cell killing activity. In some cases, any one of more of HLA-A, HLA-B, HLA-C, HLA-E, HLA-F and HLA-G of the engineered immune cell can remain intact.

In some cases, a subunit of the endogenous MHC molecule in said engineered immune cell can be inactivated such that the endogenous MHC molecule is inactive. In some cases, B2M subunit of the endogenous MHC molecule in said engineered immune cell is inactivated. In some cases, B2M subunit of the endogenous MHC molecule in said engineered immune cell is knocked out or partially knocked out. In some cases, a killer/phagocyte inhibitor of the engineered immune cell can be overexpressed.

In some other cases, an endogenous HLA can be knocked out with co-expression of killer/phagocyte inhibitor(s). For example, B2M can be knocked out with co-expression of killer/phagocyte inhibitors. The killer/phagocyte inhibitor may suppress immune response toward the engineered immune cell. The killer/phagocyte inhibitors include, but are not limited to, HLA-E single chain trimer, HLA-G, CD47, CD24, FASL, PDL1, or functional domains thereof.

In some cases, any two or more of an endogenous immune cell antigen, an endogenous TCR, and an endogenous MHC molecule of the engineered immune cell can be inactivated. In some cases, the endogenous immune cell antigen and the endogenous TCR of the engineered immune cell can be inactivated, an endogenous TCR and an endogenous MHC molecule of the engineered immune cell can be inactivated, or the endogenous immune cell antigen and an endogenous MHC molecule of the engineered immune cell can be inactivated.

In some cases, the endogenous immune cell antigen, the endogenous TCR, and the endogenous MHC molecule of the engineered immune cell can be all inactivated.

In some cases, endogenous T cell receptors (TCRs) of the engineered immune cell is inactivated. Various methods can be used to inactivate endogenous TCRs. For example, a gene encoding a subunit of the endogenous TCR can be inactivated such that the endogenous TCR is inactivated. The gene encoding the subunit can be TCRα, TCRβ, CD3ε, CD3δ, CD3γ, or CD3ζ.

The chimeric polypeptide may or may not comprise any self-cleaving peptide flanked by the enhancer moiety and the inducible cell death moiety. The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be self-activating through self-oligomerizing. The enhancer moiety can be self-activating through self-dimerizing.

The chimeric polypeptide described herein can be a secreted protein. The chimeric polypeptide can be an intracellular protein. The chimeric polypeptide can be a transmembrane protein. The enhancer moiety or the inducible cell death moiety can be contained in an ectodomain of the transmembrane protein. The enhancer moiety or the inducible cell death moiety is contained in an endodomain of the transmembrane protein. The enhancer moiety can be contained in an endodomain of the transmembrane protein and the inducible cell death moiety can be contained in an ectodomain of the transmembrane protein. The enhancer moiety can be contained in an ectodomain of the transmembrane protein, and the inducible cell death moiety can be contained in an endodomain of the transmembrane protein.

The enhancer moiety can be a cytokine or a cytokine receptor. For example, the enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The inducible cell death moiety can be selected from the group consisting of rapaCasp9, iCasp9, HSV-TK, ΔCD20, mTMPK, ΔCD19, RQR8, and EGFRt. In some cases, the inducible cell death moiety is EGFRt, and the cell death activator is an antibody or an antigen binding fragment thereof that binds EGFRt. In some cases, the inducible cell death moiety is HSV-TK, and the cell death activator is GCV. In some cases, the inducible cell death moiety is iCasp9, and the cell death activator is AP1903. The cell death activator may comprise a nucleic acid, a polynucleotide, an amino acid, a polypeptide, lipid, a carbohydrate, a small molecule, an enzyme, a ribosome, a proteasome, a variant thereof, or any combination thereof.

The CAR T cell provided herein can be a universal CAR T cell. The CAR T cell can express a chimeric antigen receptor CAR that targets a tumor cell marker and the binding of the T cell receptor to PD-1 is inhibited. The CAR T cell can target CD7. The endogenous TCR expression in the CAR T cells provided herein can be knocked out by gene editing technology. Upon knocking out the endogenous TCRs of the CAR T cells, the normal cells may not be recognized and killed by the CAR T cells during the allogeneic infusion. The GVHD reaction may be inhibited. Moreover, targeting tumor cells through CD7 while eliminating host T cells and/or NK cells through CD7 can avoid host versus graft (HVG) and/or NK killing and improve the survival and anti-tumor effect of the allogeneic CAR T cells in the recipient. The CAR T can further comprise a suicide gene switch (e.g., an inducible cell death moiety). The CAR T cells can be inactivated or removed by turning on the suicide gene switch (e.g., binding of an activator to the inducible cell death moiety) to reduce the side effects of the CAR T cell therapy.

The engineered immune cell may comprise two different CARs, each having a different antigen binding domain target a different antigen. The engineered immune cell may comprise a single CAR, which further comprises two antigen binding domains targeting two different antigens. In some cases, a first CAR, and/or a second CAR is linked to an inducible cell death moiety and/or an enhancer moiety by a self-cleaving element. In some cases, the enhancer moiety is a cytokine or cytokine complex. Examples of cytokines or cytokine complexes include IL2, IL7, IL15, membrane-bound IL15 (mbIL15 or mb15), and a constitutive activating cytokine receptor such as an IL7 receptor (C7R). As used herein, “mbIL” and “mb” are used interchangeably to refer to a membrane-bound interleukin factor, for example, mbIL7 or mb7, and mbIL17 or mb17.

The engineered immune cell described herein may have the following characteristics: (a) the engineered immune cell expresses a CAR and/or an exogenous TCR, and the CAR and/or exogenous TCR targets tumor cell markers; and (b) the cytokine-associated signaling pathway is enhanced. The engineered immune cell may be (i) chimeric antigen receptor T cells (CAR T cells); (ii) chimeric antigen receptor NK cells (CAR-NK cells); or (iii) Exogenous T cell receptor (TCR) T cells (TCR-T cells). The engineered immune cell can be a CAR T cell, preferably a universal CAR T cell (UCAR T cell). The “cytokine-related signaling pathway,” as used herein, refers to a signaling pathway initiated by the cytokine binding to the corresponding receptor, converting the extracellular signal into an intracellular signal, which is then amplified, dispersed, and regulated by a signal cascade. A series of cellular responses can be produced. In some cases, the cytokine-related signaling pathway comprises a related signaling pathway of a cytokine selected from the group consisting of IL-2, IL-3, IL-4, IL-6, IL-7, IL-8, IL-10, IL-11, IL-12, IL-15, IL-17, IL-18, IL-21, 25 or a combination thereof.

The engineered immune cell can comprise a bispecific CAR (or a dual CAR). For example, the bispecific CAR can comprise both a first antigen binding domain and a second antigen binding domain. The first antigen binding domain and the second antigen binding domain can be linked via a linker. The linker may not comprise a self-cleaving peptide. The first antigen binding domain or the second antigen binding can be a scFv.

The first antigen binding domain and the second antigen binding domain can be arranged, from amino terminus to carboxyl terminus, in any of following patterns: (i) VL2-VH1-VL1-VH2; (ii) VH2-VL1-VH1-VL2; (iii) VL1-VH2-VL2-VH1; and (iv) VH1-VL2-VH2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain.

The first antigen binding domain and the second antigen binding domain can be arranged, from amino terminus to carboxyl terminus, in any of following patterns: (i) VL2-VH2-VL1-VH1; (ii) VL2-VH2-VH1-VL1; (iii) VL1-VH1-VL2-VH2; and (iv) VL1-VH1-VH2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. The first antigen binding domain and the second antigen binding domain can bind to the immune cell antigen and the disease-associated antigen.

In some cases, the engineered immune cell may not comprise a bispecific CAR. For example, an individual CAR of the engineered immune cell can comprise only the first antigen binding domain and an additional individual CAR of the engineered immune cell can comprise only the second antigen binding domain.

The immune cell antigen can be a surface protein or a secreted protein of an immune cell. The immune cell can be an NK cell, a T cell, a monocyte, a macrophage or a granulocyte. The immune cell antigen can be selected from the group consisting of CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ and SLAMF7.

The disease-associated antigen can be a tumor-associated antigen. The tumor-associated antigen can be CS1 or other antigens described herein. In some cases, the first antigen binding domain can bind to an immune cell antigen selected from the group consisting of CD2, CD3, CD5, CS1, CD7 and CD137, and the second antigen binding domain can bind to CS1. In some cases, the first antigen binding domain can bind to CD7, and the second antigen binding domain can bind to CS1. In some cases, the first antigen binding domain can bind to CD137, and the second antigen binding domain can bind to CS1. The expression of one or more endogenous human leukocyte antigen (HLA) genes of the engineered immune cell can remain intact. The expression of one or more of endogenous HLA-A, HLA-B, HLA-C, HLA-E, HLA-F and HLA-G genes of the engineered immune cell can remain intact. The expression of one or more endogenous human leukocyte antigen (HLA) genes of the engineered immune cell can be inactivated. The expression of one or more of endogenous HLA-A, HLA-B, HLA-C, HLA-E, HLA-F and HLA-G genes of the engineered immune cell can be inactivated. The expression of one or more endogenous human leukocyte antigen (HLA) genes of the engineered immune cell can be downregulated. The expression of one or more of endogenous HLA-A, HLA-B, HLA-C, HLA-E, HLA-F and HLA-G genes of the engineered immune cell can be downregulated. The expression of one or more endogenous human leukocyte antigen (HLA) genes of the engineered immune cell can be knockout or partially knockout.

In various embodiments, the engineered immune cell is a T cell, an NKT cell or an NK cell. In some cases, the engineered immune cell is derived from a stem cell. The stem cell can be a hematopoietic stem cell (HSC) or an induced pluripotent stem cell (iPSC).

A cell (e.g., an engineered immune cell) provided herein can comprise one or more chimeric antigen receptors (CARs) comprising a binding moiety, where the binding moiety can comprise an antigen binding domain capable of binding to an immune cell antigen. Each CAR of the one or more CARs can further comprise a transmembrane domain and an intracellular signaling region. The cell can further comprise an enhancer moiety capable of enhancing one or more activities of the cell, where an endogenous T cell receptor (TCR) of the cell may be inactivated.

The enhancer moiety can enhance one or more activities of the cell. The enhancer moiety can be configured to constitutively enhance the one or more activities of the cell. The enhancer moiety can be configured to constitutively upregulate one or more intracellular signaling pathways of the cell. For example, the one or more intracellular signaling pathways can be one or more cytokine signaling pathways. The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The cell can further comprise an inducible cell death moiety capable of effecting death of the cell upon contacting the inducible cell death moiety with a cell death activator. The inducible cell death moiety can be selected from the group consisting of rapaCasp9, iCasp9, HSV-TK, ΔCD20, mTMPK, ΔCD19, RQR8, Her2t, CD30, BCMA, and EGFRt. For example, the inducible cell death moiety can be EGFRt, and the cell death activator can be an antibody or an antigen binding fragment thereof that binds EGFRt. For another example, the inducible cell death moiety can be HSV-TK, and the cell death activator can be GCV. For another example, the inducible cell death moiety can be iCasp9, and the cell death activator can be AP1903.

A gene encoding an endogenous surface marker of the cell can be inactivated, where the endogenous surface marker may be capable of binding to the first antigen binding domain when expressed. The endogenous surface marker can be CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ or SLAMF7.

### Enhancer Moiety

The engineered immune cell provided herein can comprise an enhancer moiety. The enhancer moiety can regulate one or more activities of the engineered immune cell, for example, enhance or upregulate one or more signaling pathways to enhance or upregulate effector functions of the engineered immune cell. The signaling pathways can be a cytokine-related signaling pathway. The enhancer moiety can be a cytokine. The enhancer moiety can be a cytokine receptor.

The cytokine-related signaling pathway can comprise a related signaling pathway of a cytokine. Examples of cytokines include, but are not limited to, IL-2, IL-3, IL-4, IL-6, IL-7, IL-8, IL-10, IL-11, IL-12, IL-15, IL-17, IL-18, IL-21 and IL25. In some cases, the cytokine-related signaling pathway comprises a related signaling pathway of two or more cytokines, wherein the cytokines include: IL-2 and IL-7, IL-2 and IL-15. IL-7 and IL-15, IL15 and IL21. The cellular response can include regulation of downstream gene expression, changes in intracellular enzyme activity, changes in cellular bone architecture, changes in DNA synthesis, promotion of gene transcription, regulation of immune cell differentiation, proliferation, and resistance to cell death. In some cases, the cytokine-related signaling pathway is enhanced comprising: introducing or up-regulating a gene encoding a cytokine and/or a receptor thereof, exogenously adding a cytokine, being introduced into a cytokine receptor, or a combination thereof. In some cases, up-regulating the gene encoding the cytokine and/or its receptor comprises up-regulating the level of transcription and/or translation of the encoding gene. In some cases, the enhanced cytokine-related signaling pathway can be achieved by one or more of the following methods: expressing a gene encoding the cytokine and/or its receptor in the immune cell, increasing the copy number of the gene encoding the cytokine and/or its receptor in the immune cell, engineering a regulatory sequence (e.g., a promoter) of the encoding gene to enhance transcription speed (e.g., transcriptional initiation rate), modifying a translational regulatory region of a messenger RNA carrying the encoded gene to enhance translational strength, modifying the coding gene itself to enhance mRNA stability, protein stability and to release protein feedback inhibition.

The cytokine-related signaling pathway can be enhanced by membrane expression of a cytokine and its receptor, secretion of a cytokine, enhancement of transcriptional regulation of a cytokine and/or its receptor, or a combination thereof. The membrane-expressed cytokine and its receptors can include: IL-15 and its receptor (e.g., mbIL15 fusion protein), IL-7 and its receptor (e.g., mbIL7 fusion protein), IL-17 and its receptor (e.g., mbIL17 fusion protein), IL-2 and its receptor (e.g., mbIL2 fusion protein), IL-21 and its receptor (e.g., mbIL21 fusion protein), constitute the activated IL-7 receptor (C7R), or a combination thereof. In some cases, the enhancer moiety comprised in the engineered immune cell is a secretive cytokine. The secretive cytokine can function with various mechanisms, for example, the secretive cytokine can be a trans-activating factor or a cis-activating factor. The secretive cytokines can include IL-2, IL-3, IL-4, IL-6, IL-7, IL-8, IL-10, IL-11, IL-12, IL-15, IL-17, IL-18, IL-21, or a combination thereof. In some cases, the enhancer is a membrane bound protein such as mbIL15, mbIL7, mbIL21 and mbIL2. In some cases, the enhancer moiety is constitutively active cytokine receptor downstream signaling protein such as STAT5 and STAT3. In some cases, the enhancer moiety is a constitutively active cytokine receptor such as constitutively active IL-7 receptor (C7R) or derivatives thereof. For example, the constitutively active cytokine receptor can be an engineered protein (e.g., referred to as “E3” in the present disclosure) where the ecto domain of C7R is replaced by a safety switch, such as EGFRt or truncated form of human epidermal growth factor receptor 2 (Her2t; see U.S. Patent Application Publication No. 20170267742A1) or other peptides described in the present disclosure. For another example, the constitutively active cytokine receptor can be an engineered protein (e.g., referred to as “E4” in the present disclosure) where the ectodomain of C7R is replaced by an immune cell inhibitor, such as CD47, CD24 or other peptides that inhibit killer or phagocytic immune cell function and protect therapeutic cells (e.g., the engineered immune cells described herein). In some cases, the cytokine can be a chemokine such as CCL21 and CCL19. Other non-limiting examples of chemokines that may be used include CCL27, CCL28, CCL20, CXCL9, CXCL10, CXCL11, CXCL16, CXCL13, CXCL5, CXCL6, CXCL8, CXCL12, CCL2, CCL8, CCL13, CCL25, CCL3, CCL4, CCL5, CCL7, CCL14, CCL15, CCL16, CCL23, CX3CL1, XCL1, XCL2, CCL1, CCL17, CCL22, CCL11, CCL24, CCL26, CXCL1, CXCL2, CXCL3 and CXCL7. In some cases, the enhancer moiety is a ligand of CCR7, which can function to enhance infiltration of T cells, NK cells or dendritic cells. CCR7 ligand includes, but not limited to, CCL21 and CCL19. In some cases, the enhancer moiety comprises co-expression of chemokines CCL21 and CCL19 for therapeutic use to treat lymphomas or other solid tumors.

In some situations, the engineered immune cell is used as a therapeutic agent to treat liquid tumors, and in such situations, the enhancer moiety can comprise any cytokine in any form as described herein. In some situations, the engineered immune cell is used as a therapeutic agent to treat solid tumors, and in such situations, the enhancer moiety can comprise any cytokine in any form and further comprise one or more chemokines.

In some cases, two cytokines may be used to enhance the cytokine-related signaling pathway in the engineered immune cell, including IL-2 and IL-7, IL-2 and IL-15, IL-7 and IL-15, and IL15 and IL21. The cytokine-related signaling pathway enhancement can comprise the expression of a polypeptide selected from the group consisting of a mbIL fusion protein, a constitutively active IL-7 receptor (C7R), an interleukin, or a combination thereof.

The enhancer moiety described herein can be interleukin 15 (IL-15) or IL-15 receptor. IL-15 is a 14-15kDs glycoprotein composed of 114 amino acids and belongs to the family of four helix bundle cytokines. IL-15 is structurally homologous to interleukin 2 (IL-2). IL-15 receptor comprises a high affinity IL-15 receptor alpha chain, an IL2/15 receptor beta chain, and a common gamma chain. Therefore, IL-15 may have some functions similar to IL-2, such as stimulating T cell activation and proliferation, enhancing NK cell killing activity and promoting B cell production of immunoglobulin. Recent studies have found that IL-15 may play a role in the differentiation, proliferation and activation of NK cells, NKT cells and intestinal epithelial cells. IL-15 and IL-17 may play a role in the regulation of CD8+ memory T cells. Studies have also shown that IL-15 can regulate the proliferation of CD8+ memory T cells and the survival cycle of NK cells through a mechanism, in which a cell expressing IL-15α chain receptor can present IL-15 to a cell expressing an IL-15β chain and a common gamma chain. IL-15 may also play a role in the non-immune system, such as regulation of skeletal muscle anabolism. The enhancer moiety described herein can be interleukin 7 (IL-7). IL-7 can promote the growth of pre-B cells, pro-B cells, B cells, and T cells. It can also promote growth and anti-apoptosis of B cells and T cells. IL-7 can play a role in the early differentiation and proliferation of thymus and the development and differentiation of dendritic cells. However, IL-7 may not have an enhanced effect on the killing activity of antigen-specific cytotoxic T lymphocytes. It can first transfer from the thymus to the peripheral blood, then induce thymocytes or peripheral blood lymphocytes to produce lymphokines, activate and enhance lymphokine-activated killer cell activity of LAK cells. CD8+ subpopulation can be the main effector cell of IL-7, and IL-7 Can also support memory CD8+T cell expansion and survival. IL-7 can promote bone marrow tissue production. IL-7 not only can stimulate myeloid precursor cells and megakaryocytes to produce colony forming units and platelets, but also can restore the body from the immunosuppression of cyclophosphamide. At higher concentrations, it can also induce cytotoxicity that enhances macrophages, function as a synergistic factor for the production of CTL cells, NK cells, and activated monocytes, induce monocyte-macrophages to secrete various cytokines and promote the expression of inflammatory factors such as macrophage inflammatory protein alpha (MIP-alpha), MIP-β, IL-8 and monocyte chemoattractant protein-1 (MCP-1) and the like. By activating a large number of inflammatory factors produced by inflammatory cells, IL-7 not only can regulate the interaction between the components of the inflammatory process, but also enhance the inflammatory cytokine receptors (CCR) such as CCR1, CCR2 and CCR5. In addition, IL-7 can play a role in inducing immune responses. IL-7 can induce type I immune responses and increase the production of IFN-γ and IL2. IL-7 can synergize with IL12 to induce IFN-γ and T cell proliferation. IL-7 and transforming growth factor beta (TGFβ) can play a regulatory role and can be part of the immune regulatory mechanism. IL-7 not only can promote immune reconstitution of T cells, but also can induce up-regulation of T cell cycle and BCL-2 expression, which broadens the diversity and persistence of circulating T cell receptor pools and increases the number of CD4+ and CD8+T cells. Moreover, for HIV antigens, expanded T cells can also secrete IL2 and IFN-γ, and have good antiviral function. Therefore, IL-7 can reverse the defects of HIV-specific T lymphocytes in proliferation, cytokine secretion and cell function.

The enhance moiety can regulate (e.g., activate) signal transducer and activator of transcription 5 (STAT5)-mediated signaling pathway. STAT5 can be widely present in the cytoplasm. When cytokines (e.g., IL2, IL7, IL15 and IL21) bind to the cytokine receptors, the receptor-coupled JAK is activated, thereby phosphorylating the Tyr residue at the C-terminus of the STAT5 protein. The phosphorylated STAT5 can form homologous or heterologous dimers through its SH2 region. The homologous or heterodimer can be transferred to the nucleus and bind to the target gene, thereby regulating the expression of the target gene including the cell regulatory factor and the anti-apoptotic gene. Activation of STAT5 can play a role in maintaining normal cell function and regulating cell proliferation and differentiation. Therefore, regulating the activity of STAT5 signaling pathway may regulate the survival and persistence of CAR T cells described herein.

The enhancer moiety can be introduced into a cell (e.g., an immune cell or an engineered immune cell) by delivering a nucleic acid molecule encoding the enhancer moiety into the cell. The nucleic acid molecule can be a vector. The enhancer moiety can be a part of a fusion construct. A fusion protein or corresponding nucleic acid construct can have a structure as presented by a formula selected from: S-2A-L1-scFv-H-TM-C-CD3ζ-2A-L2-IL15-IL15Ra (A); S-2A-L1-scFv-H-TM-C-CD3ζ-2A-L2-IL15-IL15Ra-2A-L3-IL7 (B); S-2A-L1-scFv-H-TM-C-CD3ζ-2A-L2-C7R (C); S-2A-L1-scFv-H-TM-C-CD3ζ-2A-L2-IL7-IL7Ra (D); wherein: each “-” is independently a linker peptide or a peptide bond; S is a safety switch; 2A is an optional self-cleaving peptide; each of L1, L2 and L3 is independently null or a signal peptide sequence; C7R is as described above; scFv is an antigen binding domain; H is null or a hinge region; TM is a transmembrane domain; C is a costimulatory signaling molecule; CD3ζ is a intracellular signaling region as described above; IL15 is interleukin 15, IL15Ra is IL-15 receptor α; IL7 is interleukin 7, IL7Ra is IL-7 receptor α; C7R is a constitutively activated IL-7 receptor.

The enhancer moiety can be part of a chimeric polypeptide. For example, the enhancer moiety can be linked to an inducible cell death moiety. The enhancer moiety can be linked to the inducible cell death moiety by a linker. The linker may not be cleaved. The linker may not comprise a self-cleaving peptide. In some other cases, the enhancer moiety and the inducible cell death moiety can be expressed in a cell from a same nucleic acid molecule and can be cleaved to form two polypeptides.

### Inducible Cell Death Moiety

The engineered immune cell described herein may comprise an inducible cell death moiety, also referred to as “suicide gene switch.” “suicide switch,” “safety switch,” or “cell suicide element.” The inducible cell death moiety can be used to effectively remove of the engineered immune cells (e.g., CAR T cells) in vivo under the action of exogenous factors (e.g., drugs). The inducible cell death moiety described herein may be rapaCasp9, iCasp9, CD20 (and its mimotope), RQR8, Her2t, CD30, BCMA, EGFRt, HSV-TK, mTMPK and the like. iCasp9, CD20 (and its mimotope), RQR8, and HSV-TK may have the same ability to clear T cells, but rapaCasp9, iCasp9, RQR8, and CD20 (and their mimotope) may be faster in comparison with HSV-TK.

In some cases, an inducible cell death moiety is capable of effecting death of said cell upon contacting said inducible cell death moiety with a cell death activator. The inducible cell death moiety can be, for example, rapaCasp9, iCasp9, HSV-TK, ΔCD20, mTMPK, ΔCD19, RQR8, or EGFRt. In some cases, the inducible cell death moiety is EGFRt, and said cell death activator is an antibody or an antigen binding fragment thereof that binds EGFRt. In some cases, the inducible cell death moiety is HSV-TK, and said cell death activator is GCV. In some cases, the inducible cell death moiety is iCasp9, and said cell death activator is AP1903.

The inducible cell death moiety can be linked to an enhancer moiety and can be co-expressed in a cell as a chimeric polypeptide as described above.

### Graft Versus Host Disease (GVHD)

To prepare “off-the-shelf” allogeneic T cells for the treatment of malignant and infectious diseases, cell therapy by infusion of T cells can be designed to re-establish immunity against pathogens and malignancies. The amount of time required to produce the T cells with tumor-targeting properties with a sufficient number of T cells in vitro can be generally incompatible with the patient's therapeutic window. Furthermore, autologous T cells from patients with advanced disease may have impaired function and are tolerant to the desired antigen.

To address these issues, patients can be administered with allogeneic T cells but need to be prevented from immune-mediated rejection by host NK cells and T cells by recognizing different major or minor histocompatibility antigens on the infused cells. Infusion of T cells without the expression of TCR alpha and beta chains and/or MHC molecules may not cause GVHD and HVG. Thus the T cells edited with CRISPR/CAS9 to delete TCR alpha chain and/or MHC molecules can serve as a source of universal effector donor cells.

Graft Rejection

Knockdown of Beta-2-Microglobulin (B2M) may prevent donor CAR T cells from being attacked by the host T cells. The donor CAR T cells may be attacked by host NK cells and affect the survival of CAR T cells. Therefore, the present disclosure provides engineered immune cells which target tumor cells and host T cells and/or NK cells. The engineered immune cells described herein can scavenge host T cells and/or NK cells, and enhance the survival, persistence and expansion ability of CAR T cells, thereby being more effective against tumor cells. Or through expression of one or more NK cell inhibitors include, but are not limited to, HLA-E single chain trimer, HLA-G, PD-L1, FASL, PDL1, or functional domains thereof.

### Gene Editing

Various gene editing methods can be used in the present disclosure to make the engineered immune cells, including CRISPR, RNA interference technology, TALENs (transcription activator-like (TAL) effector nucleases) and Zinc finger nucleases (ZFNs).

In some cases, CRISPR/Cas9 system is used to edit the genes of the immune cells. For example, CRISPR/Cas9 system can be used to knockout endogenous TCRs or cell surface markers (e.g., CS1, CD7, CD137) of the immune cells to generate the engineered immune cells for T cell therapy. The CRISPR/Cas9 (clustered regular interspaced short palindromic repeats)/Cas (CRISPR-associated) system is a natural immune system unique to prokaryotes that is resistant to viruses or exogenous plasmids. The Type II CRISPR/Cas system has been applied in many eukaryotic and prokaryotic organisms as a direct genome-directed genome editing tool. The development of the CRISPR/Cas9 system has revolutionized the ability of people to edit DNA sequences and regulate the expression levels of target genes, providing a powerful tool for accurate genome editing of organisms. The simplified CRISPR/Cas9 system can comprise Cas9 protein and gRNA. The principle of action is that gRNA forms a Cas9-gRNA complex with Cas9 protein through its own Cas9 handle, and the base complementary pairing sequence of gRNA in the Cas9-gRNA complex is paired with the target sequence of the target gene by the principle of base complementary pairing. Cas9 uses its own endonuclease activity to cleave the target DNA sequence. Compared to traditional genome editing techniques, the CRISPR/Cas9 system has several distinct advantages: ease of use, simplicity, low cost, programmability, and the ability to edit multiple genes simultaneously.

### Pharmaceutical Composition

The present disclosure also provides a pharmaceutical composition comprising an engineered immune cell described herein and a pharmaceutically acceptable carrier, diluent or excipient. In some embodiments, the pharmaceutical composition is a liquid composition. The pharmaceutical composition can be administered into a subject, for example, by injection. The concentration of the engineered immune cells in the preparation can be at least about 10², 10³, 10⁴, 10⁵, 10⁶, 10⁷, 10⁸, 10⁹, or more cells/ml. In some case, the concentration of the engineered immune cells in the preparation can be 1×10³-1×10⁸ cells/ml, or 1×10⁴-1×10⁷ cells/ml.

The pharmaceutical compositions of the present disclosure may comprise engineered immune cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the engineered immune cells (e.g., CAR T cells) described herein may be administered at a dosage of 10⁴ to 10⁹ cells/kg body weight, or in some cases, 10⁵ to 10⁶ cells/kg body weight, including all integer values within those ranges. T cell compositions may also be administered multiple times at these dosages. The cells can be administered by using infusion techniques. The optimal dosage and treatment regime for a particular patient can readily be determined by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the T cell compositions of the present disclosure are administered to a patient by intradermal or subcutaneous injection. In some other embodiments, the T cell compositions of the present disclosure are preferably administered by i.v. injection. The compositions of T cells may be injected directly into a tumor, lymph node, or site of infection.

### Therapeutics

The present disclosure provides therapeutic applications with engineered immune cells (e.g., T cells or NK cells) transduced with a lentiviral vector (LV) encoding an expression cassette described herein. Transduced T cells or NK cells can target tumor cell markers (such as CD7, CD19, BCMA) and activated T cell and/or NK cell consensus markers (such as CD7, CD137, etc.). The engineered immune cells can be used for allogeneic tumor treatment and can be prepared on a large scale.

Accordingly, the present disclosure also provides a method of stimulating a T cell mediated immune response to a target cell population or tissue of a subject (e.g., a mammal) comprising the step of administering to the subject an engineered immune cell (e.g., CAR T cell) of the disclosure.

In some embodiments, the present disclosure provides a type of cell therapy comprising directly administering engineered universal CAR T cells of the present disclosure to a patient in need thereof. The CAR T cells of the present disclosure may have the endogenous TCR expression knocked out or silenced in the cells by gene editing technology. Inactivation of the endogenous TCRs and/or MHC (such as B2M) can prevent killing of normal cells by the TCRs during the allogeneic infusion. The GVHD reaction may be prevented. The CAR T cells targeting a tumor cell marker (such as CD7, CD19, BCMA) and a marker for activated T cells and/or NK cells (such as CD7, CD137) can remove activated T cells and/or NK cells while scavenging tumor cells. In addition, host versus graft response (HVG) can also be prevented. The cell therapy provided herein can also improve the survival and anti-tumor effect of allogeneic CAR T cells in the subject.

In some embodiments, provided herein is a method of treating or diagnosing a disease in a subject, comprising administering the pharmaceutical composition described herein to said subject.

The engineered immune cell in said pharmaceutical composition can be derived from an allogeneic immune cell. The engineered immune cell derived from said allogeneic immune cell may not induce graft versus host disease (GvHD) in said subject. The engineered immune cell in said pharmaceutical composition can be derived from an autologous immune cell.

The endogenous TCR and/or MHC (such as B2M) of said engineered immune cell in said pharmaceutical composition may be functionally inactive. The engineered immune cell can reduce GVHD in said subject compared to an additional immune cell having a functionally active TCR and/or MHC (such as B2M). The disease can be a cancer. The cancer can be, for example, lymphoma or leukemia.

The CAR T cells of the present disclosure can undergo robust in vivo cell expansion and can be extended. The CAR-mediated immune response can be part of a step of adoptive immunotherapy in which CAR-modified T cells can induce an immune response specific for the antigen-binding domain in the CAR. For example, anti-CD7 CAR T cells elicit a specific immune response against cells expressing CD7.

The engineered immune cells provided herein can be used to treat cancers. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the present disclosure include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma, craniopharyngioma, ependymoma, pinealoma, hemangioblastoma, acoustic neuroma, oligodendroglioma, meningioma, neuroblastoma, retinoblastoma and brain metastases).

The cell therapy disclosed herein can be co-formulated with, and/or co-administered with, one or more additional therapeutic agents, e.g., one or more anti-cancer agents, cytotoxic or cytostatic agents, hormone treatment, vaccines, and/or other immunotherapies. In some embodiments, the engineered immune cells are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

In certain embodiments, the methods and compositions described herein are administered in combination with one or more antibody molecules, chemotherapy, other anti-cancer therapy (e.g., targeted anti-cancer therapies, or oncolytic drugs), cytotoxic agents, immune-based therapies (e.g., cytokines), surgical and/or radiation procedures. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole body ir-radiation).

In certain embodiments, the combination therapy, is used in combination with a standard of cancer care chemotherapeutic agent including, but not limited to, anastrozole (Arimidex®), bicalu-tamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cy-tosar-U®), cytarabine liposome injection (DepoCyt®), dacarbazine (DTIC-Dome®), dactinomy-cin (Actinomycin D, Cosmegan), daunorubicin hydrochloride (Cerubidine®), daunorubicin citrate liposome injection (DaunoXome®), dexamethasone, docetaxel (Taxotere®), doxorubicin hydro-chloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeox-ycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injec-tion (Hycamptin®), vinblastine (Velban®), vincristine (Oncovin®), and vinorelbine (Navelbine®).

Examples of alkylating agents include, without limitation, nitrogen mustards, ethylenimine deriva-tives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pi-pobroman (Amedel®, Vercyte®), triethylenemelamine (Hemel®, Hexalen®, Hexastat®), triethy-lenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylal-anine mustard, Alkeran®); Altretamine (also known as hexamethylmelamine (HMM), Hexalen®); Carmustine (BiCNU®); Bendamustine (Treanda®); Busulfan (Busulfex® and Myleran®); Car-boplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of anthracyclines include, e.g., doxorubicin (Adriamycin® and Rubex®); bleomycin (Lenoxane®); daunorubicin (dauorubicin hydrochloride, daunomycin, and rubidomycin hydro-chloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetyl-ravidomycin.

Examples of vinca alkaloids that can be used in combination with the cell therapy described herein, include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Examples of proteasome inhibitors that can be used in combination with the cell therapy described herein, include, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912); danoprevir (RG7227, CAS 850876-88-9); ixazomib (MLN2238, CAS 1072833-77-2); and (S)—N-[(phenylmethoxy)carbonyl]-L-leucyl-N-(1-formyl-3-methylbutyl)-L-Leucinamide (MG-132, CAS 133407-82-6).

In some embodiments, the cell therapy may be used in combination with a Tyrosine kinase inhibitor (e.g., a receptor Tyrosine kinase (RTK) inhibitor). Exemplary Tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RE-CENTIN, AZD2171), dasatinib (SPRYCEL®, BMS-354825), erlotinib (TARCEVAR), gefitinib (IRESSAR), imatinib (Gleevec®, CGP57148B, STI-571), lapatinib (TYKERB®, TYVERB®), lestaurtinib (CEP-701), neratinib (HKI-272), nilotinib (TASIGNAR), semaxanib (semaxinib, SU5416), sunitinib (SUTENTR, SU11248), toceranib (PALLADIAR), vandetanib (ZACTIMAR, ZD6474), vatalanib (PTK787, PTK/ZK), trastuzumab (HERCEPTIN®), bevacizumab (AVAS-TIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAM-PATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Further examples of hedgehog inhibitors include, but are not limited to, vismodegib (2-chloro-N-[4-chloro-3-(2-pyridinyl)phenyl]-4-(methylsulfonyl)-benzamide, GDC-0449); 1-(4-Chloro-3-(trifluoromethyl)phenyl)-3-((3-(4-fluorophenyl)-3,4-dihydro-4-oxo-2-quinazolinyl)methyl)-urea (CAS 330796-24-2); N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-2(3H),9′(1′H)-naphth[2,1-a]azulen]-3′-yl]-methanesulfonamide (IPI926, CAS 1037210-93-7); and 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9); and Erismodegib (LDE225). Selected Tyrosine kinase inhibitors are chosen from sunitinib, erlotinib, gefitinib, or sorafenib erlotinib hydrochloride (Tarceva®); linifanib (N-[4-(3-amino-1H-indazol-4-yl)phenyl]-N′-(2-fluoro-5-methylphenyl)urea, also known as ABT 869, avail-able from Genentech); sunitinib malate (Sutent®); bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, described in U.S. Pat. No. 6,780,996); dasatinib (Sprycel®); pazopanib (Votrient®); sorafenib (Nexavar®); zactima (ZD6474); and imatinib or imatinib mesylate (Gil-Vec® and Gleevec®).

In certain embodiments, the cell therapy can be used in combination with a Vascular Endothelial Growth Factor (VEGF) receptor inhibitors, including but not limited to, Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfan-ib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

In some embodiments, the cell therapy described herein can be used in combination with a PI3K inhibitor. The PI3K inhibitor can be an inhibitor of delta and gamma isoforms of PI3K. Examples of PI3K inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine; 2-Methyl-2-[4-[3-methyl-2-oxo-8-(quinolin-3-yl)-2,3-dihydroimidazo[4,5-c]quinolin-1-yl]phenyl]propionitrile; 4-(trifluoromethyl)-5-(2,6-dimorpholinopyrimidin-4-yl)pyridin-2-amine; Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); 2-Amino-8-ethyl-4-methyl-6-(1H-pyrazol-5-yl)pyrido[2,3-d]pyrimidin-7(8H)-one (SAR 245409 or XL 765); 1,3-Dihydro-8-(6-methoxy-3-pyridinyl)-3-methyl-1-[4-(1-piperazinyl)-3-(trifluoromethyl)phenyl]-2H-imidazo[4,5-c]quinolin-2-one, (2Z)-2-butenedioate (1:1) (BGT 226); 5-Fluoro-3-phenyl-2-[(1S)-1-(9H-purin-6-ylamino)ethyl]-4(3H)-quinazolinone (CAL101); 2-Amino-N-[3-[N-[3-[(2-chloro-5-methoxyphenyl)amino]quinoxalin-2-yl]sulfamoyl]phenyl]-2-methylpropanamide (SAR 245408 or XL 147); and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (BYL719).

In some embodiments, the cell therapy described herein can be used in combination with a mTOR inhibitor, e.g., one or more mTOR inhibitors chosen from one or more of rapamycin, temsirolimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl di-methylphosphinate, also known as AP23573 and MK8669, and described in PCT Publication No. WO 03/064383); everolimus (Afinitor® or RAD001); rapamycin (AY22989, Sirolimus®); simapi-mod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), (1r,4r)-4-(4-amino-5-(7-methoxy-1H-indol-2-yl)imidazo[1,5-f][1,2,4]triazin-7-yl)cyclohexanecarboxylic acid (OSI-027); and XL765.

In some embodiments, the cell therapy can be used in combination with a BRAF inhibitor, e.g., GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7); tuvizanib (AV951, CAS 475108-18-0); vemurafenib (Zel-Boraf®, PLX-4032, CAS 918504-65-1); encorafenib (also known as LGX818); 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one ox-ime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1H-pyrrolo[2,3-b]pyridine-3-carbonyl)-2,4-difluorophenyl)propane-1-sulfonamide (also known as PLX4720).

In some embodiments, the cell therapy described herein can be used in combination with a MEK inhibitor. Any MEK inhibitor can be used in combination including, but not limited to, selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886); ARRY-142886 trametinib dimethyl sulfoxide (GSK-1120212, CAS 1204531-25-80); G02442104 (also known as GSK1120212), RDEA436; N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766); RDEA119/BAY 869766, AS703026; G00039805 (also known as AZD-6244 or selumetinib), BIX 02188; BIX 02189; 2-[(2-Chloro-4-iodophenyl)amino]-N-(cyclopropylmethoxy)-3,4-difluoro-benzamide (also known as CI-1040 or PD184352); CI-1040 (PD-184352), N-[(2R)-2,3-Dihydroxypropoxy]-3,4-difluoro-2-[(2-fluoro-4-iodophenyl)amino]-benzamide (also known as PD0325901); PD03259012′-amino-3′-methoxyflavone (also known as PD98059 available from Biaffin GmbH & Co., KG, Germany); PD98059, 2,3-bis[amino[(2-aminophenyl)thio]methylene]-butanedinitrile (also known as U0126); U0126, XL-518 (also known as GDC-0973, Cas No. 1029872-29-4, available from ACC Corp.); GDC-0973 (Methanone, [3,4-difluoro-2-[(2-fluoro-4-iodophenyl)amino]phenyl][3-hydroxy-3-(25)-2-piperidinyl-1-azetidinyl]-), G-38963; and G02443714 (also known as AS703206), or a pharmaceutically acceptable salt or solvate thereof. Further examples of MEK inhibitors include, but are not limited to, benimetinib (6-(4-bromo-2-fluorophenylamino)-7-fluoro-3-methyl-3H-benzoimidazole-5-carboxylic acid (2-hydroxyethyoxy)-amide, also known as MEK162, CAS 1073666-70-2); 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, the cell therapy described herein can be used in combination with a JAK2 inhibitor, e.g., CEP-701, INCB18424, CP-690550 (tasocitinib). Example JAK inhibitors include, but are not limited to, ruxolitinib (Jakafi®); tofacitinib (CP690550); axitinib (AG013736, CAS 319460-85-0); 5-Chloro-N2-[(1S)-1-(5-fluoro-2-pyrimidinyl)ethyl]-N4-(5-methyl-1H-pyrazol-3-y)-12,4-pyrimidinediamine (AZD1480, CAS 935666-88-9); (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6); momelotinib (CYT 387); baricitinib (INCB-028050 or LY-3009104); pacritinib (SB1518); (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317); gandotinib (LY 2784544); and N,N-cicyclopropyl-4-[(1,5-dimethyl-1H-pyrazol-3-yl)amino]-6-ethyl-1,6-dihydro-1-methyl-imidazo[4,5-d]pyrrolo[2,3-b]pyridine-7-carboxamide (BMS 911543).

In some embodiments, the combination therapies disclosed herein include paclitaxel or a paclitaxel agent, e.g., TAXOL®, protein-bound paclitaxel (e.g., ABRAXANE®). Exemplary paclitaxel agents include, but are not limited to, nanoparticle albumin-bound paclitaxel (ABRAX-ANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by Im-munoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel me-thyl 2-glucopyranosyl succinate).

### Methods

The present disclosure provides methods for generating an engineered cell. In some embodiments, the method can comprise (a) delivering a nucleic acid molecule expressing a chimeric polypeptide into a cell; and (b) expressing the nucleic acid molecule in the cell, thereby generating the engineered cell. The chimeric polypeptide can be a chimeric antigen receptor as described herein.

The present disclosure also provides methods for administering an engineered cell as described herein. The engineered cell can be an engineered immune cell. The engineered immune cell can be a T cell. The engineered immune cell can be derived from an autologous T cell. The engineered immune cell can be derived from an allogeneic T cell.

In some embodiments, provided herein is a method for administering an engineered immune cell comprising a chimeric polypeptide comprising (i) an enhancer moiety capable of enhancing one or more activities of the engineered immune cell, and (ii) an inducible cell death moiety capable of effecting death of the engineered immune cell upon contacting the chimeric polypeptide with a cell death activator. The enhancer moiety can be linked to the inducible cell death moiety. The engineered immune cell can further comprise one or more chimeric antigen receptors (CARs) comprising a binding moiety. The binding moiety can comprise a first antigen binding domain, which first antigen binding domain suppresses or reduces a subject's immune response toward the engineered immune cell when administered into the subject. The binding moiety can further comprise a second antigen binding domain capable of binding to a disease-associated antigen. An individual CAR of the one or more CARs can comprise (i) the first antigen binding domain, (ii) the second antigen binding domain, or (iii) both the first antigen binding domain and the second antigen binding domain. Each CAR of the one or more CARs can further comprise a transmembrane domain and an intracellular signaling region.

In some embodiments, provided herein is a method of administering an engineered immune cell comprising one or more chimeric antigen receptors (CARs) comprising a binding moiety. The binding moiety can comprise a first antigen binding domain capable of binding to an immune cell antigen and a second antigen binding domain capable of binding to a disease-associated antigen. Each CAR of the one or more CARs can further comprise a transmembrane domain and an intracellular signaling region. The engineered immune cell can further comprise an enhancer moiety capable of enhancing one or more activities of the engineered immune cell. In some cases, an endogenous T cell receptor (TCR) of the engineered immune cell can be inactivated. The engineered immune cell can exhibit (i) enhanced degree of persistence by remaining viable in vitro for at least about 5, 10, 15, 20, 25, 30, 35, 40, 45, 50, 55, 60, 65, 70, 75, 80, 85, 90, 95, 100, 150, 200, 250, 300, 350, 400, 450, 500, or more days while in presence of cells (e.g., cancer cells, immune cells, or both) that are heterologous to the engineered immune cell, (ii) enhanced degree of expansion by at least about 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 200-fold, 250-fold, 300-fold, or more within 15 days, or (iii) enhanced cytotoxicity against a target cell comprising the immune cell antigen or the disease-associated antigen, compared to an additional engineered immune cell comprising the one or more CARs but not the enhancer moiety. In some cases, the engineered immune cell can exhibit enhanced degree of expansion by at least about 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 350-fold, 400-fold, 450-fold, 500-fold, or more within 30 days. In some cases, the engineered immune cell can exhibit enhanced degree of expansion by at least about 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, 10,000-fold, 20,000-fold, 30,000-fold, 40,000-fold, 50,000-fold, 60,000-fold, 70,000-fold, 80,000-fold, 90,000-fold, 100,000-fold, 200,000-fold, 300,000-fold, 400,000-fold, 500,000-fold, 600,000-fold, 700,000-fold, 800,000-fold, 900,000-fold, 1,000,000-fold, or more within 60 days. The viability or expansion can be measured in the presence of stimulation, for example, stimulation by a cancer antigen or a cancer cell. The viability or expansion can be measured in the presence of multiple rounds or repeated stimulations.

In some embodiments, provided herein is a method of administering a cell (e.g., an engineered immune cell), comprising a functionally inactive T cell receptor (TCR). The cell can further comprise one or more chimeric antigen receptors (CARs). Each individual CAR of the one or more CARs can comprise a binding moiety. The binding moiety can comprise (i) a first antigen binding domain, which first antigen binding domain suppresses or reduces a subject's immune response toward the engineered immune cell when administered into the subject and (ii) a second antigen binding domain that binds to a disease-associated antigen. Each CAR of the one or more CARs can further comprise a transmembrane domain and an intracellular signaling region.

In some embodiments, provided herein is a method of administering an engineered immune cell comprising an enhancer moiety capable of enhancing one or more activities of the engineered immune cell. The engineered cell can further comprise a chimeric antigen receptor (CAR) comprising an antigen binding domain that specifically binds CD7. The CAR can further comprise a transmembrane domain and an intracellular signaling region. The endogenous CD7 in the engineered immune cell can be inactivated. In some embodiments, the engineered cell can comprise a CAR comprising an antigen binding domain that specifically binds an immune cell antigen. The immune cell antigen can be any immune cell antigen described herein such as CD2, CD3, CD4, CD5, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ and SLAMF7. The endogenous immune cell antigen of the engineered cell, which the antigen binding domain binds, can be inactivated in the engineered cell.

In some embodiments, provided herein is a method of administering an engineered immune cell comprising a single chimeric antigen receptor (CAR), and the single CAR comprises a first antigen binding domain and a second antigen binding domain. The CAR can further comprise a hinge domain, a transmembrane domain, and intracellular signaling region as described above.

The present disclosure also provides methods of treating or diagnosing a disease in a subject. In some cases, the method comprises administering a pharmaceutical composition comprising an engineered immune cell into a subject. The engineered immune cell in the pharmaceutical composition can be derived from an allogeneic immune cell. The engineered immune cell derived from the allogeneic immune cell may not induce graft versus host disease (GVHD) in the subject. The engineered immune cell in the pharmaceutical composition can be derived from an autologous immune cell. In some cases, an endogenous TCR of the engineered immune cell in the pharmaceutical composition is functionally inactive. The engineered immune cell can reduce GVHD in the subject compared to an immune cell having a functionally active TCR. In some cases, an endogenous MHC molecule (such as B2M) of the engineered immune cell in the pharmaceutical composition is functionally inactive. The engineered immune cell can reduce GVHD in the subject compared to an immune cell having a functionally active MHC molecule (such as B2M). The disease can be a cancer. For example, the cancer can be lymphoma or leukemia.

The present disclosure also provides a method of delivering an allogeneic cell therapy comprising administering to a subject in need thereof a population of engineered immune cells. An individual engineered immune cell of the population can comprise one or more chimeric antigen receptors (CARs) comprising a binding moiety. The binding moiety can comprise a first antigen binding domain capable of binding to an immune cell antigen. The binding moiety can further comprise a second antigen binding domain capable of binding to a disease-associated antigen. The first antigen binding domain can suppress or reduce a subject's immune response toward the engineered immune cell when administered into the subject. The engineered immune cell can further comprise an enhancer moiety capable of enhancing one or more activities of the engineered immune cell. The endogenous T cell receptor (TCR) of the engineered immune cell can be inactivated. For example, a gene encoding a subunit of TCR can be inactivated. The endogenous MHC molecule of the engineered immune cell can be inactivated. For example, a gene encoding a subunit of MHC such as B2M can be inactivated. Various gene editing methods described herein can be used to inactivate endogenous TCRs of a T cell.

In some embodiments, a method provided herein can include activation of a population of cells. In some cases, the cell used to prepare the engineered immune cell can be activated before preparing the engineered immune cell. In some cases, the engineered immune cell can be activated. Activation as used herein can refer to a process whereby a cell transitions from a resting state to an active state. This process can comprise a response to an antigen, migration, and/or a phenotypic or genetic change to a functionally active state. In some embodiments, activation can refer to the stepwise process of T cell activation. In some cases, a T cell can require one or more signals to become activated. For example, a T cell can require at least two signals to become fully activated. The first signal can occur after engagement of a TCR by the antigen-MHC complex, and the second signal can occur by engagement of costimulatory molecules. Anti-CD3 antibody (or a functional variant thereof) can mimic the first signal and anti-CD28 antibody (or a functional variant thereof) can mimic the second signal in vitro.

In some embodiments, a method provided herein can comprise activation of a population of cells. Activation can be performed by contacting a population of cells with a surface having attached thereto an agent that can stimulate a CD3 TCR complex associated signal and a ligand that can stimulate a costimulatory molecule on the surface of the cells. In particular, T cell populations can be stimulated in vitro such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For costimulation of an accessory molecule on the surface of the T cells, a ligand that binds the accessory molecule can be used. For example, a population of cells can be contacted with an anti-CD3 antibody and an anti-CD28 antibody, under conditions that can stimulate proliferation of the T cells. In some cases, 4-1BB can be used to stimulate cells. For example, cells can be stimulated with 4-1BB and IL-21 or another cytokine. For activation of either CD4 T cells or CD8 T cells, an anti-CD3 antibody and an anti-CD28 antibody can be used. For example, the agents providing a signal may be in solution or conjugated to a solid phase surface. The ratio of particles to cells may depend on particle size relative to the target cell. In further embodiments, the cells, such as T cells, can be combined with agent-coated beads, where the beads and the cells can be subsequently separated, and optionally cultured. Each bead can be coated with either anti-CD3 antibody or an anti-CD28 antibody, or in some cases, a combination of the two. In an alternative embodiment, prior to culture, the agent-coated beads and cells are not separated but are cultured together. Cell surface proteins may be conjugated by allowing paramagnetic beads to which anti-CD3 antibody and anti-CD28 antibody can be attached (3×28 beads) to contact the T cells. In one embodiment the cells and beads (for example, DYNABEADS® M-450 CD3/CD28 T paramagnetic beads at a ratio of 1:1) are combined in a buffer, for example, phosphate buffered saline (PBS) (e.g., without divalent cations such as, calcium and magnesium). Any cell concentration may be used. The mixture may be cultured for or for about several hours (e.g., about 3 hours) to or to about 14 days or any hourly integer value in between. In another embodiment, the mixture may be cultured for or for about 21 days or for up to about 21 days. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. Other additives for the growth of cells include, but are not limited to, surfactant, plasmanate, and reducing agents such as N-acetyl-cysteine and 2-mercaptoethanol. Media can include RPMI 1640, A1 M-V, DMEM, MEM, α-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, can be included only in experimental cultures, possibly not in cultures of cells that are to be infused into a subject. The target cells can be maintained under conditions necessary to support growth; for example, an appropriate temperature (e.g., 37° C.) and atmosphere (e.g., air plus 5% CO₂). In some instances, T cells that have been exposed to varied stimulation times may exhibit different characteristics. In some cases, a soluble monospecific tetrameric antibody against human CD3, CD28, CD2, or any combination thereof may be used. In some embodiments, activation can utilize an activation moiety, a costimulatory agent, and any combination thereof. In some embodiments, an activation moiety binds: a CD3/T cell receptor complex and/or provides costimulation. In some embodiments, an activation moiety is any one of anti-CD3 antibody and/or anti-CD28 antibody. In some embodiments, a solid phase is at least one of a bead, plate, and/or matrix. In some embodiments, a solid phase is a bead. Alternatively or in addition to, the activation moiety may be not be conjugated a substrate, e.g., the activation moiety may be free-floating in a medium.

In some cases, a population of cells can be activated or expanded by co-culturing with tissue or cells. A cell can be an antigen presenting cell. An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a costimulatory signal. For example, a costimulatory signal can be anti-CD28, inducible costimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof. In some cases an artificial antigen presenting cell (aAPC) may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof. In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPCs can be combined with exogenously introduced cytokines for T cell activation, expansion, or any combination. Cells can also be expanded in vivo, for example in the subject's blood after administration of genomically transplanted cells into a subject.

In some embodiments, a method provided herein can comprise transduction of a population of cells. In some embodiments, a method comprises introducing a polynucleotide encoding for a cellular receptor such as a chimeric antigen receptor and/or a T cell receptor. In some cases, a transfection of a cell can be performed.

In some embodiments, a viral supernatant comprising a polynucleotide encoding for a cellular receptor such as a CAR and/or TCR is generated. In some embodiments, a viral vector can be a retroviral vector, a lentiviral vector and/or an adeno-associated viral vector. Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV. Additional methods for the delivery of nucleic acids to cells can be used, for example, as described in US20030087817, incorporated herein by reference.

In some embodiments, a host cell can be transiently or non-transiently transfected with one or more vectors described herein. A cell can be transfected as it naturally is present in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. Non-limiting examples of vectors for eukaryotic host cells include but are not limited to: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWL-neo, pSv2cat, pOG44, pXT1, pSG (Stratagene) pSVK3, pBPv, pMSG, pSVL (Pharmiacia). Also, any other plasmids and vectors can be used as long as they are replicable and viable in a selected host. Any vector and those commercially available (and variants or derivatives thereof) can be engineered to include one or more recombination sites for use in the methods. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBIl21, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3 pREP4, pCEP4, and pEBVHis (Invitrogen, Corp.), and variants or derivatives thereof. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSYSPORTI (Invitrogen) and variants or derivatives thereof. Additional vectors of interest can also include pTrxFus, pThioHis, pLEX, pTrcHis, pTrcHis2, pRSET, pBlueBa-cHis2, pcDNA3.1/His, pcDNA3.1(−)/Myc-His, pSecTag, pEBVHis, pPIC9K, pPIC3.5K, pA081S, pPICZ, pPICZA, pPICZB, pPICZC, pGAPZA, pGAPZB, pGAPZC, pBlue-Bac4.5, pBlueBacHis2, pMelBac, pSinRep5, pSinHis, pIND, pIND(SP1), pVgRXR, pcDNA2.1, pYES2, pZEr01.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; X ExCell, X gtI1, pTrc99A, pKK223-3, pGEX-1X T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-lb(+), pT7Blue(R), pT7Blue-2, pCITE-4-abc(+), pOCUS-2, pTAg, pET-32L1C, pET-30LIC, pBAC-2 cp LIC, pBACgus-2 cp LIC, pT7Blue-2 LIC, pT7Blue-2, X SCREEN-1, X BlueSTAR, pET-3abcd, pET-7abc, pET9abcd, pET 11 abcd, pET12abc, pET-14b, pET-15b, pET-16b, pET-17b-pET-17 xb, pET-19b, pET-20b(+), pET-21abcd(+), pET-22b(+), pET-23abcd(+), pET-24abcd (+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFPN, pEGFP-C, pEBFP, pGFPuv, pGFP, p6xHis-GFP, pSEAP2-Basic, pSEAP2-Contral, pSEAP2-Promoter, pSEAP2-Enhancer, p I3 gal-Basic, pl3 gal-Control, p I3 gal-Promoter, p I3 gal-Enhancer, pCMV, pTet-Off, pTet-On, pTK-Hyg, pRetro-Off, pRetro-On, pIRESlneo, pIRESihyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, 2Xgt10, Xgtl1, pWE15, and X TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phag-escript, pCAL-n-EK, pCAL-n, pCAL-c, pCAL-kc, pET-3abcd, pET-llabcd, pSPUTK, pESP-1, pCMVLacI, pOPRSVI/MCS, pOPI3 CAT, pXT1, pSG5, pPbac, pMbac, pMClneo, pMClneo Poly A, pOG44, p0G45, pFRTI3GAL, pNE0I3GAL, pRS403, pRS404, pRS405, pRS406, pRS413, pRS414, pRS415, and pRS416 from Stratagene, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp, and variants or derivatives thereof. In some embodiments, a vector can be a minicircle vector. A vector provided herein can be used to deliver a polypeptide coding for a CAR and/or TCR.

Transduction and/or transfection can be performed by any one of: non-viral transfection, biolistics, chemical transfection, electroporation, nucleofection, heat-shock transfection, lipofection, microinjection, or viral transfection. In some embodiments a provided method comprises viral transduction, and the viral transduction comprises a lentivirus. Viral particles can be used to deliver a viral vector comprising a polypeptide sequence coding for a cellular receptor into a cell ex vivo or in vivo. In some cases, a viral vector as disclosed herein may be measured as pfu (plaque forming units). In some cases, the pfu of recombinant virus or viral vector of the compositions and methods of the disclosure may be about 10⁸ to about 5×10¹⁰ pfu. In some cases, recombinant viruses of this disclosure are at least about xo 10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, the viral vector of the disclosure may be measured as vector genomes. In some cases, recombinant viruses of this disclosure are 1×10¹⁰ to 3×10¹² vector genomes, or 1x 109 to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some cases, a viral vector provided herein can be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵ to 1×10⁷. In some cases, the MOI may be 1×10⁴ to 1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI, or are at most about Ix 101, 1×10², 1x 103, 1x 104, 1x 105, 1×10⁶, 1x 107, Ix 108, 1x 109, 1x 1010, 1×10¹¹, IX 10¹², 1×10¹³, 1×10¹⁴, 1×10¹⁵, 1×10¹⁶, 1×10¹⁷, and 1×10¹⁸ MOI. In some cases, a viral vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell.

The transfection efficiency of cells with any of the nucleic acid delivery platforms described herein, for example, transduction, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, a method can comprise adding an infective agent to a composition comprising a population of cells. An infective agent can comprise polybrene. In some embodiments, an infective agent can enhance efficiency of viral infection. An infective agent can enhance viral infectivity from about 100 to 1,000 fold. Polybrene can be added to a composition at a concentration from about 5 ug to 10 ug per ml.

In some embodiments, a method provided herein can comprise a non-viral approach of introducing a cellular receptor to a cell. Non-viral approaches can include but are not limited to: CRISPR associated proteins (Cas proteins, e.g., Cas9), Zinc finger nuclease (ZFN), Transcription Activator-Like Effector Nuclease (TALEN), Argonaute nucleases, and meganucleases. Nucleases can be naturally existing nucleases, genetically modified, and/or recombinant. Non-viral approaches can also be performed using a transposon-based system (e.g. PiggyBac, Sleeping beauty).

In some embodiments, a method provided herein can utilize a PiggyBac system to introduce an exogenous polypeptide to a cell. A PiggyBac system comprises two components, a transposon and a transposase. The PiggyBac transposase facilitates the integration of the transposon specifically at ‘TTAA’ sites randomly dispersed in the genome. The predicted frequency of ‘TTAA’ in the genome is approximately 1 in every 256 base-pairs of DNA sequence. Unlike other transposons, the PB transposase also enables the excision of the transposon in a completely seamless manner, leaving no sequences or mutations behind. Furthermore, PiggyBac offers a large cargo-carrying capacity (over 200 kb has been demonstrated) with no known upper limit. PB performance levels can be increased by codon-optimization strategies, mutations, deletions, additions, substitutions, and any combination thereof. In some cases, PB can have a larger cargo (approximately 9.1-14.3 kb), a higher transposition activity, and its footprint-free characteristic can make it appealing as a gene editing tool. In some embodiments, PB can comprise a few features: high efficiency transposition; large cargo; steady long-term expression; the trans-gene is integrated as a single copy; tracking the target gene in vivo by a noninvasive mark instead of traditional method such as PCR; easy to determine the integration site, and combinations thereof.

In some embodiments, a method provided herein can utilize a Sleeping Beauty (SB) System to introduce a polypeptide coding for a cellular receptor to a cell. SB was engineered from ancient Tel/mariner transposon fossils found within the Salmonid genomes by in vitro evolution. The SB ITRs (230 bp) contain imperfect direct repeats (DRs) of 32 bp in length that can serve as recognition signals for the transposase. Binding affinity and spacing between the DR elements within ITR has involved in transpositional activities. The SB transposase can be a 39 kDa protein that possess DNA binding polypeptide, a nuclear localization signal (NLS) and the catalytic domain, featured by a conserved amino acid motif (DDE). Various screens mutagenizing the primary amino acid sequence of the SB transposase resulted in hyperactive transposase versions. In some cases, a modified SB can be utilized. Modified SBs can contain mutations, deletions and additions within ITRs of the original SB transposon. Modified SBs can comprise: pT2, pT3, pT2B, pT4, SB100X, and combinations thereof. Non-limited examples of modified SBs can be selected from: SB10, SB11 (3-fold higher than SB10), SB12 (4-fold higher than SB10), HSB1-HSB5 (up to 10-fold higher than SB10), HSB13-HSB17 (HSB17 is 17-fold higher than SB10), SB100X (100-fold higher than SB10), SB150X (130-fold higher than SB10), and any combination thereof. In some cases, SB100X is 100-fold hyperactive compared to the originally resurrected transposase (SB10). In some embodiments, SB transposition excision leaves a footprint (3 bp) at the cargo site. Integration is present into TA dinucleotides of the genome, and results in target site duplications, generated by the host repair machinery. In some cases, SB appears to possess a nearly unbiased, close-to-random integration profile. Transposon integration can be artificially targeted (A10%) to a predetermined genomic locus in wildtype systems, however in chimeric systems provided herein, SB transposon integration can be directed to a predetermined locus with efficiencies over 10%.

In some embodiments, a non-viral approach may be taken to introduce an exogenous polynucleic acid to a population of cells. In some embodiments, a non-viral vector or nucleic acid may be delivered without the use of a virus and may be measured according to the quantity of nucleic acid. Generally, any suitable amount of nucleic acid can be used with the compositions and methods of this disclosure. In some cases, nucleic acid may be at least about 1 μg, 10 μg, 100 μg, 1 μg, 10 μg, 100 μg, 200 μg, 300 μg, 400 μg, 500 μg, 600 μg, 700 μg, 800 μg, 900 μg, 1 μg, 10 μg, 100 μg, 200 μg, 300 μg, 400 ig, 500 ig, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some cases, nucleic acid may be at most about 1 μg, 10 μg, 100 μg, 1 μg, 10 μg, 100 μg, 200 μg, 300 μg, 400 μg, 500 μg, 600 μg, 700 μg, 800 μg, 900 μg, 1 μg, 10 μg, 100 μg, 200 g, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some embodiments, a non-viral approach of introducing a CAR and/or TCR sequence to a cell can include electroporation. Electroporation can be performed using, for example, the Neon® Transfection System (ThermoFisher Scientific) or the AMAXA® Nucleofector (AMAXA® Biosystems). Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some embodiments, electroporation pulse voltage may be varied to optimize transfection efficiency and/or cell viability. In some cases, the electroporation voltage may be less than about 500 volts. In some cases, the electroporation voltage may be at least about 500 volts, at least about 600 volts, at least about 700 volts, at least about 800 volts, at least about 900 volts, at least about 1000 volts, at least about 1100 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 2100 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts. In some cases, the electroporation pulse voltage required for optimal transfection efficiency and/or cell viability may be specific to the cell type. For example, an electroporation voltage of 1900 volts may optimal (e.g., provide the highest viability and/or transfection efficiency) for macrophage cells. In another example, an electroporation voltage of about 1350 volts may optimal (e.g., provide the highest viability and/or transfection efficiency) for Jurkat cells or primary human cells such as T cells. In some cases, a range of electroporation voltages may be optimal for a given cell type. For example, an electroporation voltage between about 1000 volts and about 1300 volts may optimal (e.g., provide the highest viability and/or transfection efficiency) for human 578T cells. In some cases, a primary cell can be a primary lymphocyte. In some cases, a population of primary cells can be a population of lymphocytes.

In some embodiments, electroporation pulse width may be varied to optimize transfection efficiency and/or cell viability. In some cases, the electroporation pulse width may be less than about 5 milliseconds. In some cases, the electroporation width may be at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds. In some cases, the electroporation pulse width required for optimal transfection efficiency and/or cell viability may be specific to the cell type. For example, an electroporation pulse width of 30 milliseconds may optimal (e.g., provide the highest viability and/or transfection efficiency) for macrophage cells. In another example, an electroporation width of about 10 milliseconds may optimal (e.g., provide the highest viability and/or transfection efficiency) for Jurkat cells. In some cases, a range of electroporation widths may be optimal for a given cell type. For example, an electroporation width between about 20 milliseconds and about 30 milliseconds may optimal (e.g., provide the highest viability and/or transfection efficiency) for human 578T cells.

In some embodiments, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses. In some cases, the number of electroporation pulses required for optimal transfection efficiency and/or cell viability may be specific to the cell type. For example, electroporation with a single pulse may be optimal (e.g., provide the highest viability and/or transfection efficiency) for macrophage cells. In another example, electroporation with a 3 pulses may be optimal (e.g., provide the highest viability and/or transfection efficiency) for primary cells. In some cases, a range of electroporation widths may be optimal for a given cell type. For example, electroporation with between about 1 to about 3 pulses may be optimal (e.g., provide the highest viability and/or transfection efficiency) for human cells.

In some cases, the starting cell density for electroporation may be varied to optimize transfection efficiency and/or cell viability. In some cases, the starting cell density for electroporation may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells. In some cases, the starting cell density for electroporation required for optimal transfection efficiency and/or cell viability may be specific to the cell type. For example, a starting cell density for electroporation of 1.5×10⁶ cells may optimal (e.g., provide the highest viability and/or transfection efficiency) for macrophage cells. In another example, a starting cell density for electroporation of 5×10⁶ cells may optimal (e.g., provide the highest viability and/or transfection efficiency) for human cells. In some cases, a range of starting cell densities for electroporation may be optimal for a given cell type. For example, a starting cell density for electroporation between of 5.6×10⁶ and 5×10⁷ cells may optimal (e.g., provide the highest viability and/or transfection efficiency) for human cells such as T cells.

A method for treating a lymphoid malignancy is provided. The method can comprise administering to a patient in need thereof a population of engineered immune cells. An individual engineered immune cell of the population can comprise one or more chimeric antigen receptors (CARs) comprising a binding moiety, where the binding moiety can comprise an antigen binding domain capable of binding to an immune cell antigen, and where each CAR of the one or more CARs can further comprise a transmembrane domain and an intracellular signaling region. An individual engineered immune cell of the population can further comprise an enhancer moiety capable of enhancing one or more activities of the engineered immune cell. An endogenous T cell receptor (TCR) of the engineered immune cell may be inactivated. In some cases, the number of affected cells in peripheral blood or the number of affected cells in bone marrow of the patient can be reduced by at least about 10%, 15%, 20%, 25%, 30%, 35%, 40%, 45%, 50%, 55%, 60%, 65%, 70%, 75%, 80%, 85%, 90%, 95% or more within a period (e.g., 3 weeks) after a last dosing of the engineered immune cells. In some cases, the period after a last dosing of the engineered immune cell can be about 1 week, 2 weeks, 3 weeks, 4 weeks, 5 weeks or more. The number of any one or more of autologous T cell, granulocyte, and NK cell in peripheral blood of the patient can start to increase within a period (e.g., 3 weeks) after a last dosing of the engineered immune cells. In some cases, the period after a last dosing of the engineered immune cell can be about 1 week, 2 weeks, 3 weeks, 4 weeks, 5 weeks or more.

The enhancer moiety can enhance one or more activities of the engineered immune cell. The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The engineered immune cell can further comprise an inducible cell death moiety capable of effecting death of the cell upon contacting the inducible cell death moiety with a cell death activator. The inducible cell death moiety can be selected from the group consisting of rapaCasp9, iCasp9, HSV-TK, ΔCD20, mTMPK, ΔCD19, RQR8, Her2t, CD30, BCMA, and EGFRt. For example, the inducible cell death moiety can be EGFRt, and the cell death activator can be an antibody or an antigen binding fragment thereof that binds EGFRt. For another example, the inducible cell death moiety can be HSV-TK, and the cell death activator can be GCV. For another example, the inducible cell death moiety can be iCasp9, and the cell death activator can be AP1903.

A gene encoding an endogenous surface marker of the cell can be inactivated. The endogenous surface marker can be capable of binding to the first antigen binding domain when expressed. The endogenous surface marker can be CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ or SLAMF7.

The number of any one or more of autologous T cell, granulocyte, and NK cell in peripheral blood of the patient may start to increase before the number of affected cells in peripheral blood or the number of affected cells in bone marrow is reduced by at least about 10%, 15%, 20%, 25%, 30%, 35%, 40%, 45%, 50%, 55%, 60%, 65%, 70%, 75%, 80%, 85%, 90%, 95% or more. The number of any one or more of autologous T cell, granulocyte, and NK cell in peripheral blood may start to increase after the number of affected cells in peripheral blood or the number of affected cells in bone marrow is reduced by at least about 10%, 15%, 20%, 25%, 30%, 35%, 40%, 45%, 50%, 55%, 60%, 65%, 70%, 75%, 80%, 85%, 90%, 95% or more.

### EXAMPLES

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 1 Design of the CAR T Cells

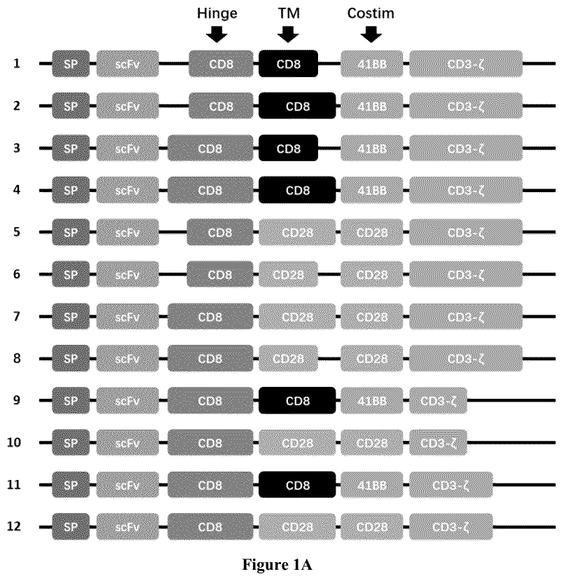

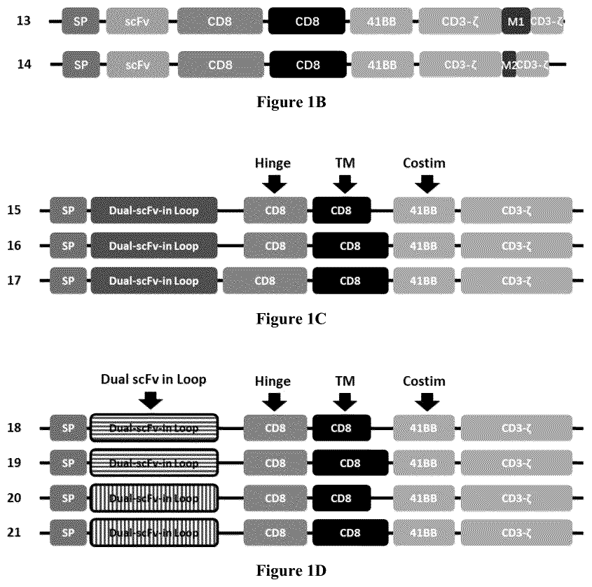

Design of the structures of CARs is as exemplarily illustrated in FIG. 1A, FIG. 1B, FIG. 1C, and FIG. 1D. In FIG. 1A, FIG. 1B, FIG. 1C, and FIG. 1D, scFv fragment represents the heavy chain and light chain variable region of CD7 monoclonal antibody (as illustrated in SEQ ID No. 16), Dual-scFv-in Loop represents CD7-CD19 dual scFvs in loop structure, also included are: (i) native CD8 hinge region (as illustrated in SEQ ID No. 18) or extended CD8 hinge region (as illustrated in SEQ ID No. 17), (ii) native CD8 transmembrane domain (TM) (as illustrated in SEQ ID No. 20) or extended CD8 transmembrane domain (TM) (as illustrated in SEQ ID No. 19), native CD28 transmembrane domain (TM) (as illustrated in SEQ ID No. 22) or truncated CD28 transmembrane domain (TM) (as illustrated in SEQ ID No. 21), (iii) 4-1ng costimulatory domain (as illustrated in SEQ ID No. 23) or CD28 costimulatory domain (as illustrated in SEQ ID No. 24), and (iv) native CD3 intracellular signaling region (as illustrated in SEQ ID No. 15) or truncated CD3 intracellular signaling region (as illustrated in SEQ ID No. 11 and 14), and optionally (v) loss of function insertion (Ml as illustrated in SEQ ID No. 33 or M2 as illustrated in SEQ ID No. 34 in FIG. 1B) in tandem.

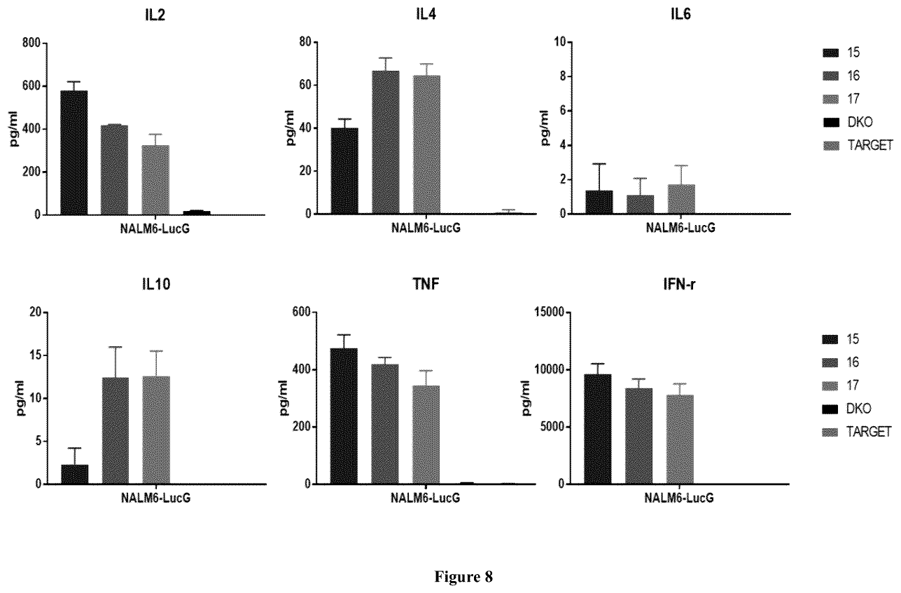

In particular, the structures of the CARs in FIG. 8A, Figure s, Figure C, and FIG. 1D are as illustrated in Table 1.

TABLE 1
Exemplary design of CAR structures of the present application

| # of | | SEQ |
|---|---|---|
| CAR | Structure | ID No. |
| 1 | CD7 scFv-native CD8 Hinge-native CD8 TM-4-1BB costim-native | 27 |
| | CD3ζ | |
| 2 | CD7 scFv- native CD8 Hinge- extended CD8 TM-4-1BB costim- | 28 |
| | native CD3ζ | |
| 3 | CD7 scFv-extended CD8 Hinge- native CD8 TM-4-1BB costim- | 29 |
| | native CD3ζ | |
| 4 | CD7 scFv-extended CD8 Hinge-extended CD8 TM-4-1BB costim- | 30 |
| | native CD3ζ | |
| 5 | CD7 scFv- native CD8 Hinge- native CD28 TM-CD28 costim- native | 31 |
| | CD3ζ | |
| 6 | CD7 scFv- native CD8 Hinge- truncated CD28 TM-CD28 costim- | 32 |
| | native CD3ζ | |
| 7 | CD7 scFv- extended CD8 Hinge- native CD28 TM-CD28 costim- | 33 |
| | native CD3ζ | |
| 8 | CD7 scFv- extended CD8 Hinge-truncated CD28 TM-CD28 costim- | 34 |
| | native CD3ζ | |
| 9 | CD7 scFv- extended CD8 Hinge- extended CD8 TM-4-1BB costim- | 35 |
| | truncated CD3ζ (SEQ ID No. 14) | |
| 10 | CD7 scFv- extended CD8 Hinge- native CD28 TM-CD28 costim- | 36 |
| | truncated CD3ζ (SEQ ID No. 14) | |
| 11 | CD7 scFv- extended CD8 Hinge-extended CD8 TM-4-1BB costim- | 37 |
| | truncated CD3ζ (SEQ ID No. 11) | |
| 12 | CD7 scFv- extended CD8 Hinge- native CD28 TM-CD28 costim- | 38 |
| | CD3ζ truncated CD3ζ (SEQ ID No. 11) | |
| 13 | CD7 scFv-extended CD8 Hinge-extended CD8 TM-4-1BB costim- | 39 |
| | CD3ζ with M1 insertion | |
| 14 | CD7 scFv-extended CD8 Hinge-extended CD8 TM-4-1BB costim- | 40 |
| | CD3ζ with M2 insertion | |
| 15 | CD7-CD19 dual scFv in Loop-native CD8 Hinge-native CD8 TM-4- | 46 |
| | 1BB costim-native CD3ζ | |
| 16 | CD7-CD19 dual scFv in Loop - native CD8 Hinge- extended CD8 | 47 |
| | TM-4-1BB costim- native CD3ζ | |
| 17 | CD7-CD19 dual scFv in Loop -extended CD8 Hinge-extended CD8 | 48 |
| | TM-4-1BB costim- native CD3ζ | |
| 18 | CD7-CD19 dual scFv (with non-humanized anti-CD19 scFv and the | 49 |
| | orientation of CD19-VL CD7-VH CD7-VL CD19-VH) in Loop-native | |
| | CD8 Hinge-native CD8 TM-4-1BB costim-native CD3ζ | |
| 19 | CD7-CD19 dual scFv (with non-humanized anti-CD19 scFv and the | 50 |
| | orientation of CD19-VL CD7-VH CD7-VL CD19-VH) in Loop-native | |
| | CD8 Hinge-extended CD8 TM-4-1BB costim-native CD3ζ | |
| 20 | CD7-CD19 dual scFv (non-humanized anti-CD19 scFv and the | 51 |
| | orientation of dual scFv is CD19-VH CD7-VH CD7-VL CD19-VL) in | |
| | Loop-native CD8 Hinge-native CD8 TM-4-1BB costim-native CD3ζ | |
| 21 | CD7-CD19 dual scFv (non-humanized anti-CD19 scFv and the | 52 |
| | orientation of dual scFv is CD19-VH CD7-VH CD7-VL CD19-VL) in | |
| | Loop-native CD8 Hinge-extended CD8 TM-4-1BB costim-native | |
| | CD3ζ | |

The CAR gene was cloned into the pCCL lentiviral vector backbone under the promoter of MND to form pCCL-MND-CAR. pCCL-MND-CAR, lentiviral envelope plasmid pMD2.G (Addgene, Plasmid #12259) and lentiviral packaging plasmid psPAX2 (Addgene, Plasmid #12260) were transfected into 293 T cells by using Lipofectamine3000 to prepare the whole lentiviral expression vector. The supernatant was collected at 48 and 72 h and subject to ultra-centrifuge (Merck Millipore) for concentration. The concentrated virus was ready for the transfection of T cells.

### Example 2 Design and Construction of CRISPR

CD7 and TCR gRNAs for gene editing were selected after evaluating gene editing efficiency and off-target risk at website http://crispr.mit.edu, www.idtdna.com and https://www.synthego.com. gRNAs and HiFi-Cas9 protein used in the present disclosure were purchased from Integrated DNA Technologies, Inc (IDT).

For gene editing, 3 μg Cas9 protein and 1.5 μg gRNA were mixed in 20 μl and incubated at 37° C. or room temperature for 15 minutes to form ribonucleoprotein (RNP). Then RNP was transfected into T cells by Lonza 4D Nucleofector. Gene knockout efficiency was determined by the ratio of the cells expressing the protein as detected by flow cytometry (FACS).

Our previous study indicated that TRAC-gRNA1 has a higher knockout efficiency for TCR. When gRNA of CD7 and TRAC-gRNA were used together to knockout CD7 and TCR simultaneously, three of four CD7-gRNAs showed high efficiencies. CD7-gRNA1 was selected as the gRNA to knockout CD7.

TRAC-gRNA1:
SEQ ID No. 41
TTCGGAACCCAATCACTGAC,;
CD7-gRNA1:
SEQ ID No. 42
GAGGTCAATGTCTACGGCTC,.

### Example 3 Preparation of CAR T Cells

Cell Isolation and Activation

After apheresis, monocytes were isolated using Histopaque-1077 (Sigma-Aldrich) by density gradient centrifugation. T cells were then enriched, activated by magnetic beads coupled with anti-CD3/anti-CD28, cultured and expanded.

X-vivo 15 with 5% FBS, 2 mM L-glutamine, 1 mM sodium pyruvate and 3001U/ml rhIL2 was used as UCAR T cell medium. The cells were incubated and cultured at 37° C., 5% CO₂.

Electroporation and Lentivirus Infection

Two days after enriching and activating the T cells, the anti-CD3/anti-CD28 magnetic beads were removed and the cells were collected into a tube and subject to centrifuge at 300 g for 5 min, and then washed twice with DPBS and re-suspended in opti-mem at a cell density of 50×10⁶/ml. The amount of cas9/gRNA RNP required was calculated based on the density of the cells, and then mixed with cells and transferred for electroporation by 4D-Nucleofector System N (Lonza) system. Then the cells were re-suspended in a pre-warmed medium to a density of 2×10⁶/ml. The cells were further subject to lentiviral transfection at MOI of 2-8, transferred to a flask and cultured in an incubator at 37° C., 5% CO₂.

Cell Proliferation and Detection of CAR Positive Ratio

3 days after the transfection, the number of cells and CAR positive cells were detected, and the positive ratio of CAR in T cells was calculated.

### Example 4 Killing Activities of CAR T Cells of the Present Application Against CCRF CEM Cells

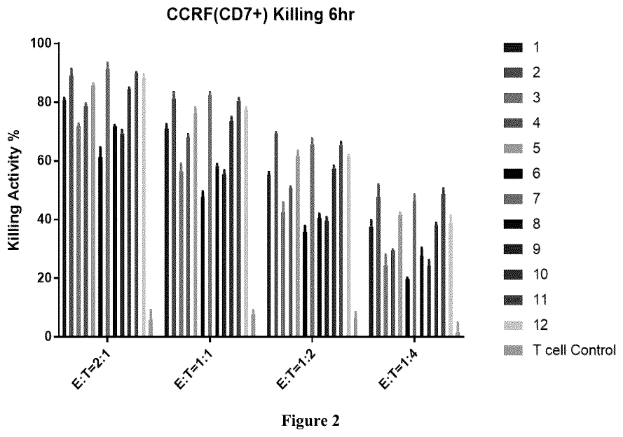

CAR T candidates or control T cells (TCR/CD7 double knockout T cells) were mixed with 4×10⁴ CCRF CEM cell in each well at E:T ratios as shown in FIG. 2, then the mixtures were cultured for 6 hours. Killing of CCRF CEM cells were measured by luciferase intensity. The result is as shown FIG. 2.

From the result, it can be seen that, at all the tested E:T ratios, #2, #5, #7 and #11 CAR T cells displayed stronger killing activities against CCRF CEM than #1 CAR T cells and control T cells, indicating improved efficacy of CAR T cells by the modified CAR T structures.

### Example 5 Cytokine Release by CAR T Cells of the Present Application

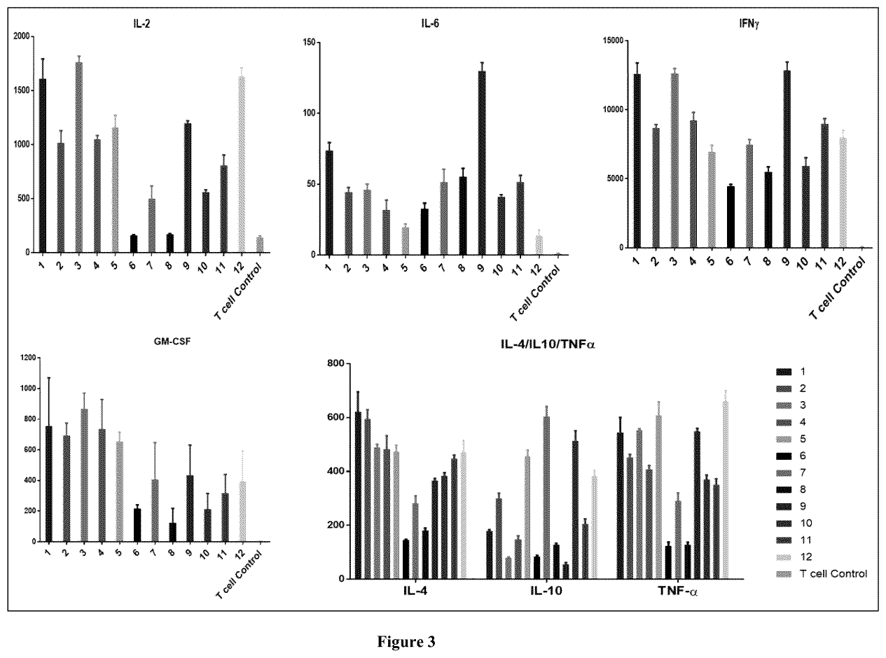

CAR T candidates or control T cells (TCR/CD7 double knockout T cells) were mixed with 4×10⁴ CCRF CEM cells in each well at E:T ratio of 1:1, and then the mixture was cultured for 24 hours. Cell culture supernatant was collected, and cell cytokines including IL-2, IL-6, IL-4, IL-10, TNFα, GM-CSF and INF-γ were analyzed by CBA assay or ELISA. The result is as shown in FIG. 3.

From the result, it can be seen that #2, #5, #7 and #11 CAR T cells produced less cytokines such as proinflammatory cytokine IL2, IL6, GM-CSF, TNF-α, IFN-γ than #1 CAR T cells, indicating less CRS risk by the modified CAR T structures.

### Example 6 In Vivo Anti-Tumor Activities of the CAR T Cells of the Present Application in Mice

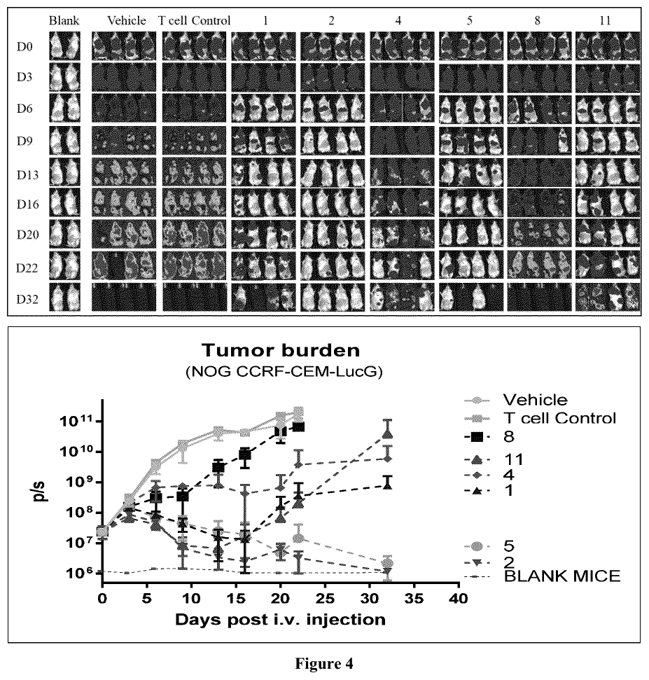

Each mouse was inoculated intravenously with 3×10⁵ tumor cells. 4 days later, CAR T cells were intravenously infused (3×10⁵ cells per mouse). BLI imaging result in FIG. 4 shows that, #2, #5 and #11 CAR T cells displayed potent in vivo anti-tumor efficacy as compared to #1 and T cell control, indicating the improved CAR T functions by the modified CAR structures.

### Example 7 Expression of Dual CAR of the Present Application in T Cells

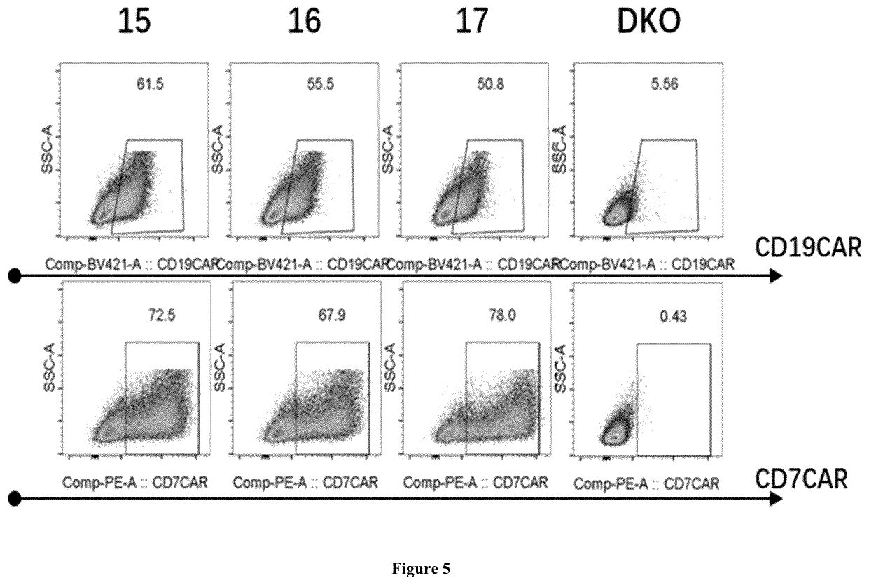

Expression of dual CAR T candidates on TCR/CD7 double knockout T cells were measured by flow cytometry, as shown in FIG. 5.

From FIG. 5, it can be seen that, #15, #16 and #17 dual CARs were all successfully expressed on the T cells, whereas the expression level of #16 and #17 dual CAR structures were comparative as #15 dual CAR structure on T cells.

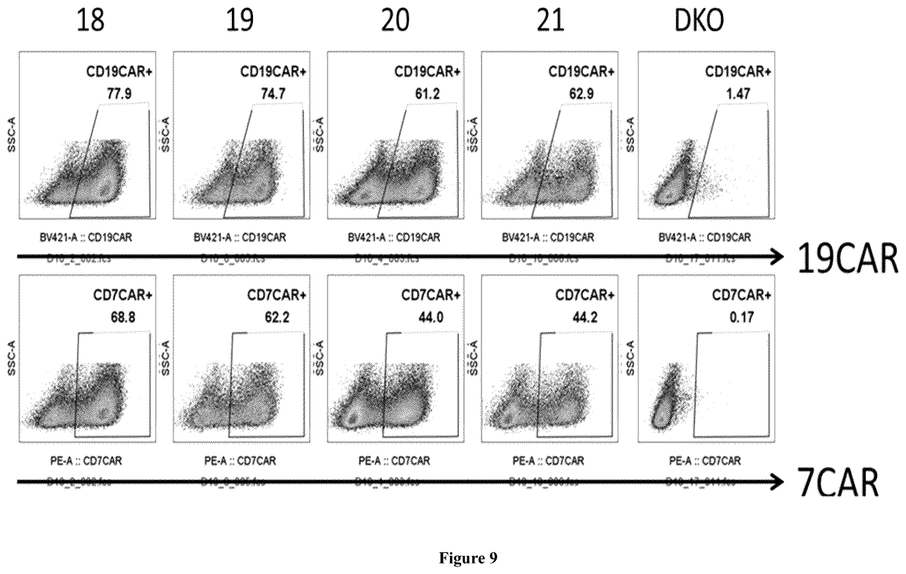

Expression of dual CAR T cells comprising the modified structures of 18-21 were measured by flow cytometry, as shown in FIG. 9. It can be seen that, #18, #19, #20, and #21 dual CARs were all successfully expressed on the T cells. Eight days after transduction, the CAR T cells exhibited binding to CD7 antigen and CD19 antigen.

### Example 8 Killing Activities of Dual CAR T Cells of the Present Application Against Hela Cells

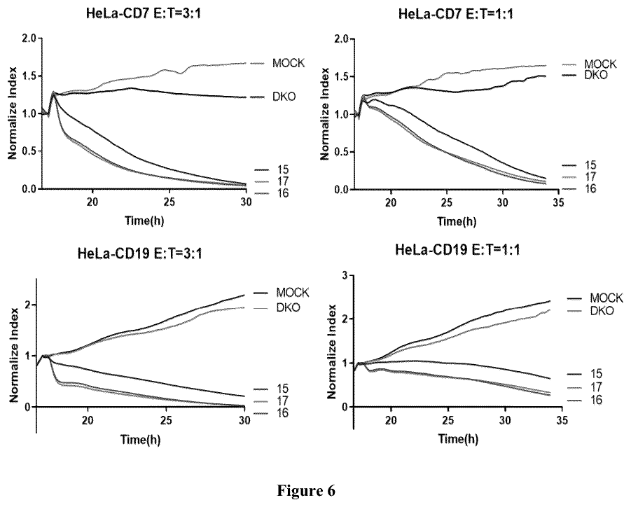

Dual CAR T candidates or control T cells (TCR/CD7 double knockout T cells) were mixed with HeLa-CD7+ cells or HeLa-CD19+ cells at E:T ratios of 3:1 or 1:1, respectively, as shown in FIG. 6. Then the killing of the HeLa-CD7+ cells and HeLa-CD19+ cells by dual CAR T cells were measure by xCELLigence Real-Time Cell Analyzer (RTCA) experiment. The result is as shown FIG. 6.

From the result, it can be seen that, at the tested E:T ratios, #15, #16 and #17 dual CAR T cells all displayed killing activities against HeLa-CD7+ cells and HeLa-CD19+ cells, whereas #16 and #17 dual CAR T cells displayed overall stronger killing activities as compared to #15 dual CAR T cells against HeLa-CD7+ cells and HeLa-CD19+ cells, indicating improved efficacy of dual CAR T cells by the modified CAR T structures.

### Example 9 Killing Activities of Dual CAR T Cells of the Present Application Against CCRF and Nalm6 Cells

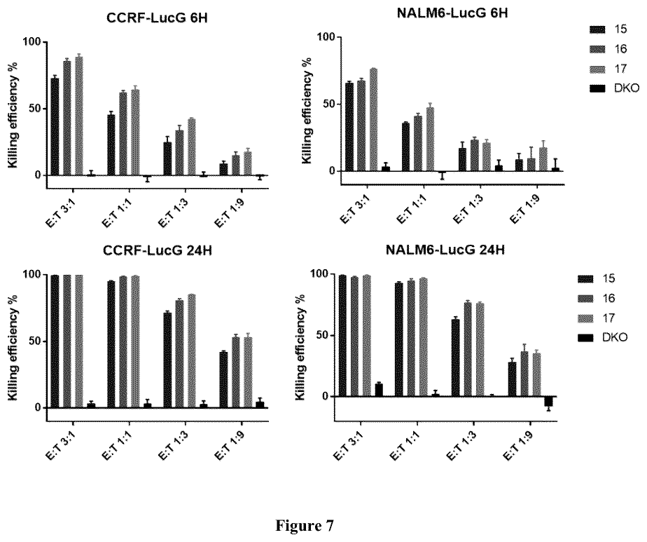

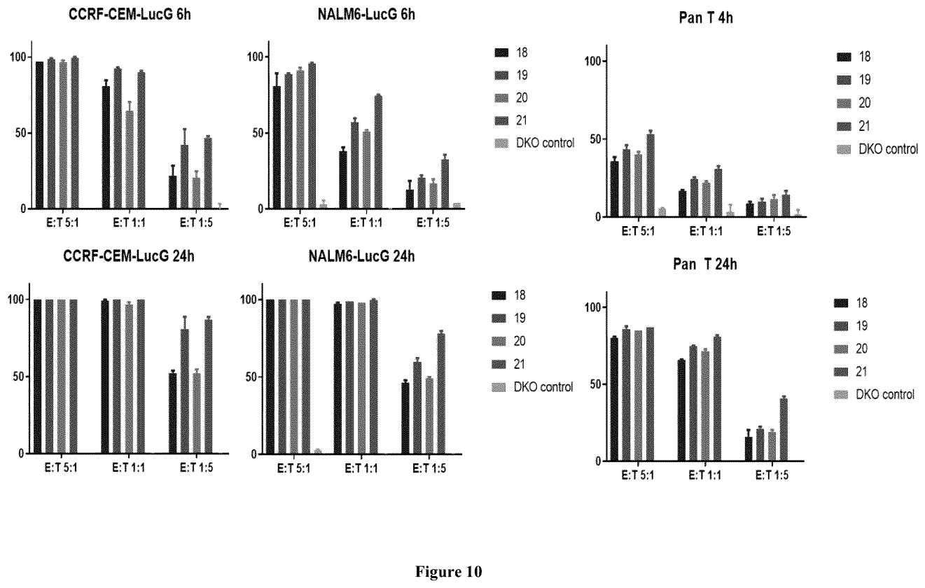

Dual CAR T candidates or control T cells (TCR/CD7 double knockout T cells) were mixed with 1×10⁴ CCRF LucG cell or NALM6-LucG cells in each well at E:T ratios of 3:1, 1:1, 1:3 and 1:9 as shown in FIG. 7, then the mixtures were cultured, and killing of CCRF LucG cells or NALM6-LucG cells were measured by luciferase intensity after 6 hours and 24 hours, respectively. The result is as shown FIG. 7 and FIG. 10.

From FIG. 7, it can be seen that, at the tested E:T ratios, #15, #16 and #17 dual CAR T cells all displayed killing activities against CCRF LucG cell or NALM6-LucG cells, whereas #16 and #17 dual CAR T cells displayed overall stronger killing activities as compared to #15 dual CAR T cells against CCRF LucG cell or NALM6-LucG cells, indicating improved efficacy of dual CAR T cells by the modified CAR T structures.

FIG. 10 illustrates cell killing efficacy of dual CAR T with modified structures 18-21 as measured from luciferase assay. CCRF-CEM cell (acute lymphoblastic leukemia cell line), Nalm6 cell (B cell precursor leukemia cell line), or primary T cell was mixed with dual CAR T cell comprising the modified structures 18-21 at a ratio that is 5:1, 1:1, or 1:5. Cell killing efficacy was measured at multiple time points. From the result, it can be seen that, at the tested E:T ratios, #18, #19, #20, and #21 dual CAR T cells all displayed killing activities against CCRF LucG cell or NALM6-LucG cells, whereas #18 and #20 dual CAR T cells displayed overall stronger killing activities as compared to #19 and #21 dual CAR T cells against CCRF LucG cell or NALM6-LucG cells.

### Example 10 Cytokine Release by Dual CAR T Cells of the Present Application

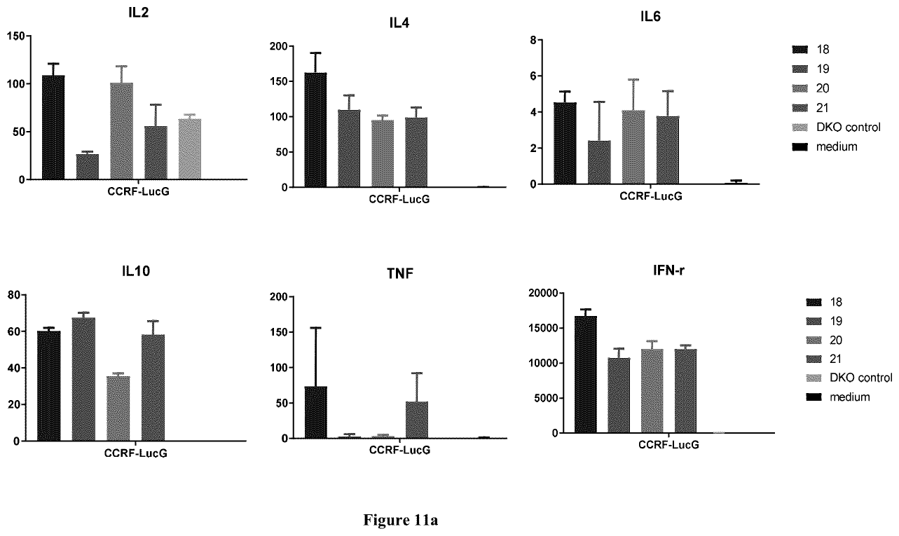

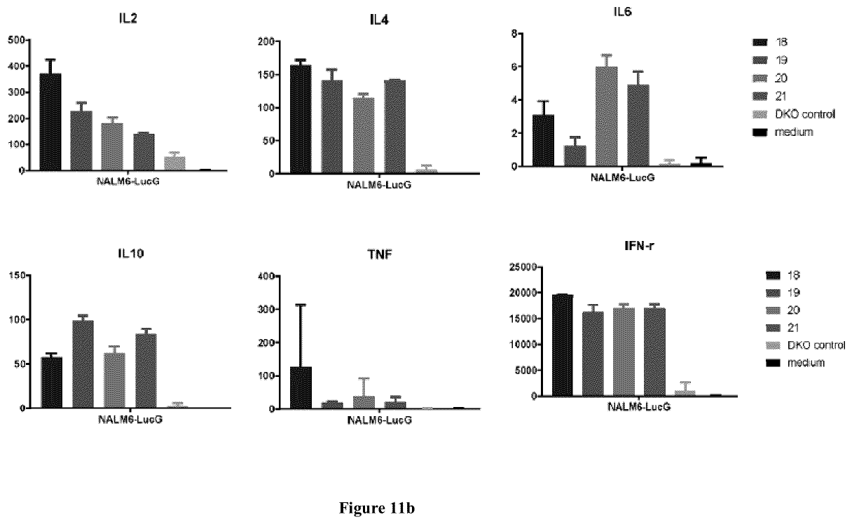

Dual CAR T candidates or control T cells (TCR/CD7 double knockout T cells) were mixed with 1×10⁴ cells NALM6-LucG cells in each well at E:T ratio of 1:1, and then the mixture was cultured for 24 hours. Cell culture supernatant was collected, and cell cytokines including IL-2, IL-4, IL-6, IL10, TNFα and IFNγ were analyzed by CBA assay. The result is as shown in FIG. 8 and FIG. 11.

From FIG. 8, it can be seen that #16, and #17 produced comparative cytokines, including IL-2, IL-4, IL-6, IL10, TNFα and IFNγ, as #15 CAR T cells, indicating safety of the modified dual CAR T structures.

FIG. 11a and FIG. 11b illustrate releases of cytokines by dual CAR T cells with modified structures 18-21 upon killing of the cancer cells (FIG. 11a, CCRF cells; and FIG. 11b, Nalm6 cells). The cytokine measurements were conducted with the CCRF-CEM cell or Nalm6 cell mixed with the dual CAR T cell at a 1:1 ratio after 24 hours. The cytokines were measured with cytometric bead array (CBA). It can be seen that dual CAR T cells with #18, #19, #20, and #21 produced comparative cytokines, including IL-2, IL-4, IL-6, IL10, TNFα and IFNγ, as #15 CAR T cells, indicating safety of the modified dual CAR T structures.

### Example 11 Proliferation of Stimulated Dual CAR T Cells

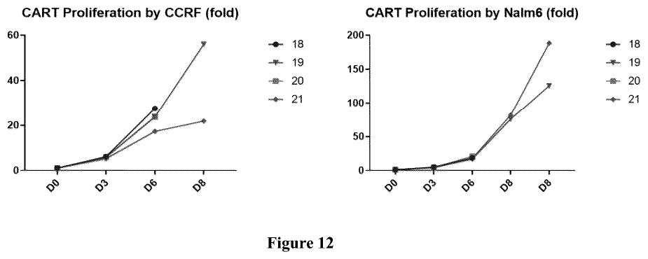

FIG. 12 illustrates proliferation of dual CAR T cell with modified structures 18-21 stimulated by mixing the dual CAR T cell with either CCRF-CEM cell or Nalm6 cell at a 1:3 ratio. The proliferation of dual CAR T cell was measured after each round of killing of the CCRF-CEM cell or Nalm6 cell. It can be seen that dual CAR T cells with #19 proliferated at a faster rate compared to dual CAR T cells with #21 when stimulated by CCRF-CEM cells. Dual CAR T cells with #21 proliferated at a faster rate compared to dual CAR T cells with #19 when stimulated by Nalm6 cells. Dual CAR T cells with #18 and Dual CAR T cells with #20 proliferated similarly when stimulated by either CCRF-CEM cells or Nalm6 cells.

### EMBODIMENTS

Embodiment 1. In one aspect, provided is an engineered immune cell, comprising: an enhancer moiety capable of enhancing one or more activities of said engineered immune cell; a chimeric antigen receptor (CAR) comprising an antigen binding domain, a hinge domain, a transmembrane domain, a costimulatory domain and a truncated CD3ζ intracellular signaling domain, wherein the truncated CD3ζ intracellular signaling domain is devoid of ITAM3 or a fragment thereof.

Embodiment 2. The engineered immune cell of Embodiment 1, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 3. The engineered immune cell of Embodiment 1, wherein the hinge domain is derived from a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

Embodiment 4. The engineered immune cell of Embodiment 1, wherein the transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

Embodiment 5. The engineered immune cell of Embodiment 1, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 6. The engineered immune cell of Embodiment 1, wherein the ITAM3 comprises an amino acid sequence of YXXL/I-X₇-YXXL/I, and each X is independently any amino acid.

Embodiment 7. The engineered immune cell of Embodiment 1, wherein the truncated CD3ζ intracellular signaling domain is devoid of an amino acid sequence of SEQ ID NO.1 or a fragment thereof.

Embodiment 8. The engineered immune cell of Embodiment 1, wherein the truncated CD3ζ intracellular signaling domain is devoid of an amino acid sequence of SEQ ID NO.2 or a fragment thereof.

Embodiment 9. The engineered immune cell of Embodiment 1, wherein the truncated CD3ζ intracellular signaling domain is devoid of an amino acid sequence of SEQ ID NO.3 or a fragment thereof.

Embodiment 10. The engineered immune cell of Embodiment 1, wherein the truncated CD3ζ intracellular signaling domain is devoid of an amino acid sequence of SEQ ID NO.4 or a fragment thereof.

Embodiment 11. The engineered immune cell of Embodiment 1, wherein the truncated CD3ζ intracellular signaling domain comprises at least ITAM1 and ITAM2.

Embodiment 12. The engineered immune cell of Embodiment 11, wherein the ITAM1 comprises an amino acid sequence of YXXL/I-X₇-YXXL/I, and each X is independently any amino acid.

Embodiment 13. The engineered immune cell of Embodiment 11, wherein the ITAM2 comprises an amino acid sequence of YXXL/I-X₈-YXXL/I, and each X is independently any amino acid.

Embodiment 14. The engineered immune cell of Embodiment 1, wherein the truncated CD3ζ intracellular signaling domain comprises an amino acid sequence of SEQ ID NO. 8.

Embodiment 15. The engineered immune cell of Embodiment 1, wherein the truncated CD3ζ intracellular signaling domain comprises an amino acid sequence of SEQ ID NO. 9.

Embodiment 16. The engineered immune cell of Embodiment 1, wherein the truncated CD3ζ intracellular signaling domain comprises an amino acid sequence of SEQ ID NO. 10.

Embodiment 17. The engineered immune cell of Embodiment 1, wherein the truncated CD3ζ intracellular signaling domain comprises an amino acid sequence of SEQ ID NO. 11.

Embodiment 18. The engineered immune cell of Embodiment 1, wherein said enhancer moiety is configured to constitutively upregulate one or more intracellular signaling pathways of said engineered immune cell.

Embodiment 19. The engineered immune cell of Embodiment 18, wherein said one or more intracellular signaling pathways are one or more cytokine signaling pathways.

Embodiment 20. The engineered immune cell of Embodiment 1, wherein said enhancer moiety is self-activating through self-oligomerizing.

Embodiment 21. The engineered immune cell of Embodiment 1, wherein said enhancer moiety is self-activating through self-dimerizing.

Embodiment 22. The engineered immune cell of Embodiment 1, wherein said enhancer moiety is a cytokine or a cytokine receptor.

Embodiment 23. The engineered immune cell of Embodiment 1, wherein said enhancer moiety is selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

Embodiment 24. The engineered immune cell of Embodiment 1, wherein said enhancer moiety functions as a trans-activating factor or a cis-activating factor.

Embodiment 25. The engineered immune cell of Embodiment 24, wherein the enhancer is linked to the CAR via a linker.

Embodiment 26. The engineered immune cell of Embodiment 25, wherein the linker is a cleavable linker.

Embodiment 27. The engineered immune cell of Embodiment 26, wherein the linker is a self-cleaving peptide.

Embodiment 28. The engineered immune cell of Embodiment 26, wherein the cleavable linker is selected from P2A, T2A, E2A, and F2A.

Embodiment 29. The engineered immune cell of Embodiment 1, wherein the engineered immune cell further comprises an inducible cell death moiety capable of effecting death of said engineered immune cell upon contacting said chimeric polypeptide with a cell death activator.

Embodiment 30. The engineered immune cell of Embodiment 29, wherein said enhancer moiety is linked to said inducible cell death moiety.

Embodiment 31. The engineered immune cell of Embodiment 29, wherein said inducible cell death moiety is selected from the group consisting of rapaCasp9, iCasp9, HSV-TK, ΔCD20, mTMPK, ΔCD19, RQR8, Her2t, CD30, BCMA and EGFRt.

Embodiment 32. The engineered immune cell of Embodiment 31, wherein said inducible cell death moiety is EGFRt, and said cell death activator is an antibody or an antigen binding fragment thereof that binds EGFRt.

Embodiment 33. The engineered immune cell of Embodiment 31, wherein said inducible cell death moiety is HSV-TK, and said cell death activator is GCV.

Embodiment 34. The engineered immune cell of Embodiment 31, wherein said inducible cell death moiety is iCasp9, and said cell death activator is AP1903.

Embodiment 35. The engineered immune cell of Embodiment 31, wherein said cell death activator comprises a nucleic acid, a polynucleotide, an amino acid, a polypeptide, lipid, a carbohydrate, a small molecule, an enzyme, a ribosome, a proteasome, a variant thereof, or any combination thereof.

Embodiment 36. The engineered immune cell of Embodiment 1, wherein the engineered immune cell is a T cell, an NKT cell or an NK cell.

Embodiment 37. The engineered immune cell of Embodiment 36, wherein the T cell is an alpha beta T cell or a gamma delta T cell.

Embodiment 38. The engineered immune cell of Embodiment 1, wherein the engineered immune cell is derived from a stem cell.

Embodiment 39. The engineered immune cell of Embodiment 38, wherein the stem cell is a hematopoietic stem cell (HSC) or an induced pluripotent stem cell (iPSC).

Embodiment 40. The engineered immune cell of Embodiment 1, wherein the engineered immune cell is an autologous cell or an allogeneic cell.

Embodiment 41. The engineered immune cell of Embodiment 1, wherein the engineered immune cell is obtained from a subject having a condition.

Embodiment 42. The engineered immune cell of Embodiment 1, wherein the engineered immune cell is obtained from a healthy donor.

Embodiment 43. In one aspect, provided is an engineered immune cell, comprising: an enhancer moiety capable of enhancing one or more activities of said engineered immune cell; a chimeric antigen receptor (CAR) comprising an antigen binding domain, a hinge domain, a transmembrane domain, a costimulatory domain and a variant of CD3ζ intracellular signaling domain, wherein the variant of CD3ζ intracellular signaling domain comprises a loss of function mutation.

Embodiment 44. The engineered immune cell of Embodiment 43, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 45. The engineered immune cell of Embodiment 43, wherein the hinge domain is derived from a polypeptide selected from the group consisting of IgG1-4, CD4, CD8, CD8α, CD8β, CD27, CD28, CD40, CD45, CD84, CD166, 4-1BB, OX40, ICOS, ICAM-1, CTLA-4, and NKGD2.

Embodiment 46. The engineered immune cell of Embodiment 43, wherein the transmembrane domain is derived from a polypeptide selected from the group consisting of CD8, CD8α, CD8β, CD4, CD27, CD28, CD40, CD45, CD84, CD166, 4-1BB, PD1, OX40, ICOS, ICAM-1, CTLA-4, and NKGD2.

Embodiment 47. The engineered immune cell of Embodiment 43, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 48. The engineered immune cell of Embodiment 43, wherein the variant of CD3ζ intracellular signaling domain comprises two loss of function mutations.

Embodiment 49. The engineered immune cell of Embodiment 43, wherein the loss of function mutation is present in an ITAM domain of CD3ζ intracellular signaling domain.

Embodiment 50. The engineered immune cell of Embodiment 49, wherein the loss of function mutation is present in ITAM1, ITAM2, or ITAM3 of CD3ζ intracellular signaling domain.

Embodiment 51. The engineered immune cell of Embodiment 49, wherein the loss of function mutation is present at Tyrosine residue of ITAM domain of CD3ζ intracellular signaling domain.

Embodiment 52. The engineered immune cell of Embodiment 51, wherein the Tyrosine residue is substituted with phenylalanine.

Embodiment 53. The engineered immune cell of Embodiment 51, wherein the loss of function mutation is present at Leucine residue of ITAM domain of CD3ζ intracellular signaling domain.

Embodiment 54. The engineered immune cell of Embodiment 43, wherein the loss of function mutation is present in a non-ITAM region of CD3ζ intracellular signaling domain.

Embodiment 55. In one aspect, provided is an engineered immune cell, comprising: a functionally inactive T cell receptor (TCR), and a chimeric antigen receptor (CAR) comprising an antigen binding domain, a hinge domain, a transmembrane domain, a costimulatory domain and a truncated CD3ζ intracellular signaling domain, wherein the truncated CD3ζ intracellular signaling domain is devoid of ITAM3.

Embodiment 56. In one aspect, provided is an engineered immune cell, comprising: a functionally inactive T cell receptor (TCR), and a chimeric antigen receptor (CAR) comprising an antigen binding domain, a hinge domain, a transmembrane domain, a costimulatory domain and a variant of CD3ζ intracellular signaling domain, wherein the variant of CD3ζ intracellular signaling domain comprises a loss of function mutation.

Embodiment 57. In one aspect, provided is an engineered immune cell, comprising: an enhancer moiety capable of enhancing one or more activities of said engineered immune cell; a chimeric antigen receptor (CAR) comprising an antigen binding domain that specifically binds to CD7, a hinge domain, a transmembrane domain, a costimulatory domain and a truncated CD3ζ intracellular signaling domain, wherein the truncated CD3ζ intracellular signaling domain is devoid of ITAM3.

Embodiment 58. In one aspect, provided is an engineered immune cell, comprising: an enhancer moiety capable of enhancing one or more activities of said engineered immune cell; a chimeric antigen receptor (CAR) comprising an antigen binding domain that specifically binds to CD7, a hinge domain, a transmembrane domain, a costimulatory domain and a variant of CD3ζ intracellular signaling domain, wherein the variant of CD3ζ intracellular signaling domain comprises a loss of function mutation.

Embodiment 59. In one aspect, provided is a chimeric antigen receptor (CAR) comprising an antigen binding domain, a CD8 hinge domain, a CD8 transmembrane domain, a costimulatory domain and a truncated CD3ζ intracellular signaling domain, wherein the CD8 hinge domain comprises an amino acid sequence of SEQ ID No. 17, the CD8 transmembrane domain comprises an amino acid sequence of SEQ ID No. 19, and the truncated CD3ζ intracellular signaling domain is devoid of ITAM3 or a fragment thereof.

Embodiment 60. The engineered immune cell of Embodiment 59, wherein the truncated CD3ζ intracellular signaling domain comprises an amino acid sequence of SEQ ID NO. 11.

Embodiment 61. The engineered immune cell of Embodiment 59, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 62. The engineered immune cell of Embodiment 59, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 63. The engineered immune cell of Embodiment 62, wherein the antigen binding domain binds to CD7.

Embodiment 64. In one aspect, provided is a chimeric antigen receptor (CAR) comprising an antigen binding domain, a CD8 hinge domain, a CD8 transmembrane domain, a costimulatory domain and a variant of CD3ζ intracellular signaling domain, wherein the CD8 hinge domain comprises an amino acid sequence of SEQ ID No. 17, the CD8 transmembrane domain comprises an amino acid sequence of SEQ ID No. 19.

Embodiment 65. The engineered immune cell of Embodiment 64, and the variant of CD3ζ intracellular signaling domain comprises a loss of function mutation

Embodiment 66. The engineered immune cell of Embodiment 64, wherein the variant of CD3ζ intracellular signaling domain comprises two loss of function mutations.

Embodiment 67. The engineered immune cell of Embodiment 64, wherein the loss of function mutation is present in an ITAM domain of CD3ζ intracellular signaling domain.

Embodiment 68. The engineered immune cell of Embodiment 67, wherein the loss of function mutation is present in ITAM1, ITAM2, or ITAM3 of CD3ζ intracellular signaling domain.

Embodiment 69. The engineered immune cell of Embodiment 67, wherein the loss of function mutation is present at Tyrosine residue of ITAM domain of CD3ζ intracellular signaling domain.

Embodiment 70. The engineered immune cell of Embodiment 69, wherein the Tyrosine residue is substituted with phenylalanine.

Embodiment 71. The engineered immune cell of Embodiment 67, wherein the loss of function mutation is present at Leucine residue of ITAM domain of CD3ζ intracellular signaling domain.

Embodiment 72. The engineered immune cell of Embodiment 64, wherein the loss of function mutation is present in a non-ITAM region of CD3ζ intracellular signaling domain.

Embodiment 73. The engineered immune cell of Embodiment 64, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 74. The engineered immune cell of Embodiment 64, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 75. The engineered immune cell of Embodiment 74, wherein the antigen binding domain binds to CD7.

Embodiment 76. In one aspect, provided is a chimeric antigen receptor (CAR) comprising an antigen binding domain, a CD8 hinge domain, a CD8 transmembrane domain, a 4-1BB costimulatory domain and a truncated CD3ζ intracellular signaling domain, wherein the CD8 hinge domain comprises an amino acid sequence of SEQ ID No. 17, and the CD8 transmembrane domain comprises an amino acid sequence of SEQ ID No. 19.

Embodiment 77. The engineered immune cell of Embodiment 76, wherein the truncated CD3ζ intracellular signaling domain is devoid of ITAM3 or a fragment thereof.

Embodiment 78. The engineered immune cell of Embodiment 76, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 79. The engineered immune cell of Embodiment 78, wherein the antigen binding domain binds to CD7.

Embodiment 80. In one aspect, provided is a chimeric antigen receptor (CAR) comprising an antigen binding domain, a CD8 hinge domain, a CD8 transmembrane domain, a 4-1BB costimulatory domain and a variant of CD3ζ intracellular signaling domain, wherein the CD8 hinge domain comprises an amino acid sequence of SEQ ID No. 17, and the CD8 transmembrane domain comprises an amino acid sequence of SEQ ID No. 19, and the variant of CD3ζ intracellular signaling domain comprises a loss of function mutation.

Embodiment 81. The engineered immune cell of Embodiment 80, wherein the variant of CD3ζ intracellular signaling domain comprises two loss of function mutations.

Embodiment 82. The engineered immune cell of Embodiment 81, wherein the loss of function mutation is present in an ITAM domain of CD3ζ intracellular signaling domain.

Embodiment 83. The engineered immune cell of Embodiment 82, wherein the loss of function mutation is present in ITAM1, ITAM2, or ITAM3 of CD3ζ intracellular signaling domain.

Embodiment 84. The engineered immune cell of Embodiment 82, wherein the loss of function mutation is present at Tyrosine residue of ITAM domain of CD3ζ intracellular signaling domain.

Embodiment 85. The engineered immune cell of Embodiment 84, wherein the Tyrosine residue is substituted with phenylalanine.

Embodiment 86. The engineered immune cell of Embodiment 82, wherein the loss of function mutation is present at Leucine residue of ITAM domain of CD3ζ intracellular signaling domain.

Embodiment 87. The engineered immune cell of Embodiment 80, wherein the loss of function mutation is present in a non-ITAM region of CD3ζ intracellular signaling domain.

Embodiment 88. The engineered immune cell of Embodiment 80, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 89. The engineered immune cell of Embodiment 88, wherein the antigen binding domain binds to CD7.

Embodiment 90. In one aspect, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

Embodiment 91. The engineered immune cell of Embodiment 90, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 92. The engineered immune cell of Embodiment 90, wherein the CD3ζ intracellular signaling domain is a truncated CD3ζ intracellular signaling domain or a variant of CD3ζ intracellular signaling domain.

Embodiment 93. The engineered immune cell of Embodiment 90, wherein the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 94. The engineered immune cell of Embodiment 93, wherein the first antigen binding domain binds to CD7.

Embodiment 95. The engineered immune cell of Embodiment 90, wherein the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 96. The engineered immune cell of Embodiment 90, wherein said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

Embodiment 97. The engineered immune cell of Embodiment 90, wherein said first antigen binding domain and said second antigen binding domain are arranged, from amino terminus to carboxyl terminus, in one of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

Embodiment 98. The engineered immune cell of Embodiment 90, wherein the CAR further comprises a second hinge domain, a second transmembrane domain, a second costimulatory domain and a second CD3ζ intracellular signaling domain.

Embodiment 99. The engineered immune cell of Embodiment 98, wherein the second hinge domain is derived from a polypeptide selected from the group consisting of CD8, CD8α, CD8β, CD27, CD28, CD40, CD45, CD84, CD166, 4-1BB, OX40, ICOS, ICAM-1, CTLA-4, and NKGD2.

Embodiment 100. The engineered immune cell of Embodiment 99, wherein the second hinge domain is derived from CD8.

Embodiment 101. The engineered immune cell of Embodiment 100, wherein the second hinge domain comprises an amino acid sequence of SEQ ID No. 17.

Embodiment 102. The engineered immune cell of Embodiment 98, wherein the second transmembrane domain is derived from a polypeptide selected from the group consisting of CD8, CD8a, CD8$, CD4, CD27, CD28, CD40, CD45, CD84, CD166, 4-1BB, PD1, OX40, ICOS, ICAM-1, CTLA-4, and NKGD2.

Embodiment 103. The engineered immune cell of Embodiment 102, wherein the second transmembrane domain is derived from CD8.

Embodiment 104. The engineered immune cell of Embodiment 103, wherein the second transmembrane domain comprises an amino acid sequence of SEQ ID No. 19.

Embodiment 105. The engineered immune cell of Embodiment 98, wherein the second costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 106. The engineered immune cell of Embodiment 98, wherein the second CD3ζ intracellular signaling domain is a truncated CD3ζ intracellular signaling domain or a variant of CD3ζ intracellular signaling domain.

Embodiment 107. The engineered immune cell of Embodiment 90, wherein the first antigen binding domain and the second antigen binding domain are linked via a linker.

Embodiment 108. The engineered immune cell of Embodiment 107, wherein the linker is a cleavable linker.

Embodiment 109. The engineered immune cell of Embodiment 107, wherein the linker is a self-cleaving peptide.

Embodiment 110. The engineered immune cell of Embodiment 107, wherein the cleavable linker is selected from P2A, T2A, E2A, and F2A.

Embodiment 111. The engineered immune cell of Embodiment 90, wherein the engineered immune cell further comprises an enhancer moiety capable of enhancing one or more activities of said engineered immune cell.

Embodiment 112. The engineered immune cell of Embodiment 90, wherein the engineered immune cell further comprises further comprises an inducible cell death moiety capable of effecting death of said engineered immune cell upon contacting said chimeric polypeptide with a cell death activator.

Embodiment 113. In one aspect, provided is an engineered immune cell comprising a chimeric antigen receptor (CAR), wherein the CAR comprises an antigen binding domain that specifically binds to CD7, a CD8 hinge domain, a CD8 transmembrane domain, a costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

Embodiment 114. The engineered immune cell of Embodiment 113, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 115. The engineered immune cell of Embodiment 114, wherein the costimulatory domain is derived from 4-1BB.

Embodiment 116. The engineered immune cell of Embodiment 113, wherein the CD3ζ intracellular signaling domain is a truncated CD3ζ intracellular signaling domain or a variant of CD3ζ intracellular signaling domain.

Embodiment 117. In one aspect, provided is an engineered immune cell comprising a chimeric antigen receptor (CAR), wherein the CAR comprises an antigen binding domain, a CD8 hinge domain, a CD8 transmembrane domain, a 4-1BB costimulatory domain and a CD3ζ intracellular signaling domain, wherein the CD8 hinge domain comprises an amino acid sequence of SEQ ID No. 17, and the CD8 transmembrane domain comprises an amino acid sequence of SEQ ID No. 19.

Embodiment 118. The engineered immune cell of Embodiment 117, wherein the CD3ζ intracellular signaling domain is a truncated CD3ζ intracellular signaling domain or a variant of CD3ζ intracellular signaling domain.

Embodiment 119. The engineered immune cell of Embodiment 117, wherein the CAR comprises a first antigen binding domain and a second antigen binding domain.

Embodiment 120. The engineered immune cell of Embodiment 119, wherein the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 121. The engineered immune cell of Embodiment 119, wherein the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 122. The engineered immune cell of Embodiment 117, wherein said first antigen binding domain and said second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

Embodiment 123. The engineered immune cell of Embodiment 117, wherein said first antigen binding domain and said second antigen binding domain are arranged, from amino terminus to carboxyl terminus, in one of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; and (viii) VH1-VL1-VH2-VL2, wherein VH1 is heavy chain variable domain of said first antigen binding domain, VL1 is light chain variable light domain of said first antigen binding domain, VH2 is heavy chain variable domain of said second antigen binding domain, and VL2 is light chain variable domain of said second antigen binding domain.

Embodiment 124. The engineered immune cell of Embodiment 117, wherein the first antigen binding domain and the second antigen binding domain are linked via a linker.

Embodiment 125. The engineered immune cell of Embodiment 117, wherein the CAR further comprises a second hinge domain, a second transmembrane domain, a second costimulatory domain and a second CD3ζ intracellular signaling domain.

Embodiment 126. In one aspect, provided is an engineered immune cell comprising a chimeric antigen receptor (CAR), wherein the CAR comprises an antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 18, the CD8 transmembrane domain comprises an amino acid sequence of SEQ ID No. 19.

Embodiment 127. The engineered immune cell of Embodiment 126, wherein the CD3ζ intracellular signaling domain is a truncated CD3ζ intracellular signaling domain or a variant of CD3ζ intracellular signaling domain.

Embodiment 128. The engineered immune cell of Embodiment 126, wherein the antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor β, GPC2, CD70, BAFF-R, TROP-2, and 4-1BB.

Embodiment 129. The engineered immune cell of Embodiment 128, wherein the antigen binding domain binds to CD7.

Embodiment 130. The engineered immune cell of Embodiment 126, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

Embodiment 131. The engineered immune cell of Embodiment 130, wherein the costimulatory domain is derived from 4-1BB.

Embodiment 132. In one aspect, provided is an engineered immune cell comprising a chimeric antigen receptor (CAR), wherein the CAR comprises a first antigen binding domain and a second antigen binding domain, a CD8 hinge domain, a CD8 transmembrane domain, a costimulatory domain and a CD3ζ intracellular signaling domain, wherein the CD8 hinge domain comprises an amino acid sequence of SEQ ID No. 18, and the CD8 transmembrane domain comprises an amino acid sequence of SEQ ID No. 19.

1-132. (canceled)

133. An engineered immune cell comprising a chimeric antigen receptor (CAR),
wherein the CAR comprises a first antigen binding domain and a second antigen binding domain, a CD8 hinge domain, a CD8 transmembrane domain, a costimulatory domain and a CD3ζ intracellular signaling domain,
wherein the CD8 hinge domain comprises (i) the amino acid sequence of SEQ ID NO. 18 and (ii) one or more amino acid modifications as compared to the amino acid sequence of SEQ ID NO. 17, and
wherein the CD8 transmembrane domain comprises the amino acid sequence of SEQ ID NO. 19.

134. The engineered immune cell of claim 133, wherein the one or more amino acid modifications comprises a plurality of amino acid deletions.

135. The engineered immune cell of claim 133, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

136. The engineered immune cell of claim 135, wherein the costimulatory domain is derived from 4-1BB.

137. The engineered immune cell of claim 133, wherein the CD3ζ intracellular signaling domain comprises the amino acid sequence of SEQ ID NO. 15 or SEQ ID NO. 45.

138. The engineered immune cell of claim 133, wherein the CD3ζ intracellular signaling domain (i) is a truncated CD3ζ intracellular signaling domain or (2) comprises at least one insertion to reduce signaling activity of the CD3ζ intracellular signaling domain.

139. The engineered immune cell of claim 138, wherein the at least one insertion is disposed between (i) ITAM1 and ITAM2 of the CD3ζ intracellular signaling domain or (ii) ITAM2 and ITAM3 of the CD3ζ intracellular signaling domain.

140. The engineered immune cell of claim 133, wherein the first antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, TROP-2, and 4-1BB.

141. The engineered immune cell of claim 140, wherein the first antigen binding domain binds to CD7.

142. The engineered immune cell of claim 133, wherein the second antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, TROP-2, and 4-1BB.

143. The engineered immune cell of claim 142, wherein the second antigen binding domain binds to CD19.

144. The engineered immune cell of claim 133, wherein the first antigen binding domain and the second antigen binding domain are arranged, from amino terminus to carboxyl terminus, in one of following patterns:
(i) VL2-VH1-VL1-VH2;
(ii) VH2-VL1-VH1-VL2;
(iii) VL1-VH2-VL2-VH1;
(iv) VH1-VL2-VH2-VL1;
(v) VL2-VL1-VH1-VH2;
(vi) VH2-VH1-VL1-VL2;
(vii) VL1-VL2-VH2-VH1; or
(viii) VH1-VH2-VL2-VL1;
wherein VH1 is a heavy chain variable domain of the first antigen binding domain, VL1 is a light chain variable domain of the first antigen binding domain, VH2 is a heavy chain variable domain of the second antigen binding domain, and VL2 is a light chain variable domain of the second antigen binding domain.

145. The engineered immune cell of claim 144, wherein the first antigen binding domain and the second antigen binding domain are arranged, from amino terminus to carboxyl terminus, VH1-VH2-VL2-VL1.

146. The engineered immune cell of claim 133, wherein the engineered immune cell exhibits enhanced cytotoxicity against target cells compared to a control immune cell, wherein the control immune cell comprises the first antigen binding domain, the second antigen binding domain, the CD8 hinge domain, a CD8 transmembrane domain that does not comprise of the amino acid sequence of SEQ ID NO. 19, the costimulatory domain, and the CD3ζ intracellular signaling domain.

147. An engineered immune cell comprising a chimeric antigen receptor (CAR),
wherein the CAR comprises an antigen binding domain that specifically binds to CD7, a CD8 hinge domain, a CD8 transmembrane domain, a costimulatory domain and a CD3ζ intracellular signaling domain,
wherein the CD8 hinge domain comprises the amino acid sequence of SEQ ID NO. 18, and the CD8 transmembrane domain comprises the amino acid sequence of SEQ ID NO. 19.

148. The engineered immune cell of claim 147, wherein the costimulatory domain is derived from a polypeptide selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

149. The engineered immune cell of claim 148, wherein the costimulatory domain is derived from 4-1BB.

150. The engineered immune cell of claim 147, wherein the CD3ζ intracellular signaling domain comprises the amino acid sequence of SEQ ID NO. 15 or SEQ ID NO. 45.

151. The engineered immune cell of claim 147, wherein the CD3ζ intracellular signaling domain (i) is a truncated CD3ζ intracellular signaling domain or (2) comprises at least one insertion to reduce signaling activity of the CD3ζ intracellular signaling domain.

152. The engineered immune cell of claim 151, wherein the at least one insertion is disposed between (i) ITAM1 and ITAM2 of the CD3ζ intracellular signaling domain or (ii) ITAM2 and ITAM3 of the CD3ζ intracellular signaling domain.

153. The engineered immune cell of claim 147, wherein the CAR further comprises an additional antigen binding domain, wherein the addition antigen binding domain binds to an antigen selected from the group consisting of CS1, CD19, CD2, CD3, CD4, CD5, CD7, CD8, CD20, CD22, CD25, CD28, CD30, CD33, CD38, CD40, CD44V6, CD47, CD52, CD56, CD57, CD58, CD79b, CD80, CD86, CD81, CD123, CD133, CD151, CD171, CD276, CLL1, B7H4, BCMA, VEGFR-2, EGFR, GPC3, PMSA, CEACAM6, c-Met, EGFRvIII, ErbB2/HER2, ErbB3, HER-2, HER3, ErbB4/HER-4, EphA2, IGF1R, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, ROR1, TNFRSF4, TWEAK-R, LTPR, LIFRP, LRP5, MUC1, MUC16, TCRα, TCRb, TLR7, TLR9, PTCH1, WT-1, Robol, Frizzled, OX40, Notch-1-4, APRIL, MAGE3, Claudin 18.2, Folate receptor α, Folate receptor 3, GPC2, CD70, BAFF-R, TROP-2, and 4-1BB.

154. The engineered immune cell of claim 153, wherein the additional antigen binding domain binds to CD19.

155. The engineered immune cell of claim 153, wherein the antigen binding domain that specifically binds to CD7 and the additional antigen binding domain are arranged, from amino terminus to carboxyl terminus, in one of following patterns:
(i) VL2-VH_{CD7}-VL_{CD7}-VH2;
(ii) VH2-VL_{CD7}-VH_{CD7}-VL2;
(iii) VL_{CD7}-VH2-VL2-VH_{CD7};
(iv) VH_{CD7}-VL2-VH2-VL_{CD7};
(v) VL2-VL_{CD7}-VH_{CD7}-VH2;
(vi) VH2-VH_{CD7}-VL_{CD7}-VL2;
(vii) VL_{CD7}-VL2-VH2-VH_{CD7}; or
(viii) VH_{CD7}-VH2-VL2-VL_{CD7};
wherein VH_{CD7} is a heavy chain variable domain of the antigen binding domain that specifically binds to CD7, VL_{CD7} is a light chain variable domain of the antigen binding domain that specifically binds to CD7, VH2 is a heavy chain variable domain of the additional antigen binding domain, and VL2 is a light chain variable domain of the second antigen binding domain.

156. The engineered immune cell of claim 155, wherein the antigen binding domain that specifically binds to CD7 and the additional antigen binding domain are arranged, from amino terminus to carboxyl terminus, VH1-VH2-VL2-VL1.

157. The engineered immune cell of claim 153, wherein the engineered immune cell exhibits enhanced cytotoxicity against target cells compared to a control immune cell, wherein the control immune cell comprises the antigen binding domain that specifically binds to CD7, the additional antigen binding domain, the CD8 hinge domain, a CD8 transmembrane domain that does not comprise of the amino acid sequence of SEQ ID NO. 19, the costimulatory domain, and the CD3ζ intracellular signaling domain.
